(19) United States
(12) Patent Application Publication
(10) Pub. No.: US 2003/0119162 A1
(43) Pub. Date: Jun. 26, 2003

(54) Structural basis of quorum sensing signal generation and methods and therapeutic agents derived therefrom

(76) Inventors: Mair E.A. Churchill, Littleton, CO (US); Susanne B. von Bodman, Tolland, CT (US); Herbert P. Schweizer, Loveland, CO (US); Ty A. Gould, Lakewood, CO (US); Tung Thanh Hoang, Calgary (CA); Frank V. Murphy IV, Cambridge (GB); William T. Watson, Aurora, CO (US)

Correspondence Address: SHERIDAN ROSS PC, 1560 BROADWAY, SUITE 1200, DENVER, CO, 80202

(21) Appl. No.: 10/189,346

(22) Filed: Jul. 2, 2002

Related U.S. Application Data
(60) Provisional application No. 60/303,449, filed on Jul. 5, 2001.
(60) Provisional application No. 60/366,575, filed on Mar. 21, 2002.

(51) Int. Cl. G06F 19/00; G01N 33/48; G01N 33/50; C12N 9/10

(52) U.S. Cl. 435/193; 702/19

(57) ABSTRACT

The three dimensional structure of acyl-homoserine lactone synthases, and particularly EsaI and LasI, and uses thereof. Novel acyl-homoserine lactone synthases from mycobacteria, nucleic acid molecules encoding such synthases, recombinant molecules and host cells, and uses thereof.

### CROSS-REFERENCE TO RELATED APPLICATIONS

This application claims the benefit of priority under 35 U.S.C. §119(e) from U.S. Provisional Application Serial No. 60/303,449, filed Jul. 5, 2001, and from U.S. Provisional Application Serial No. 60/366,575, filed March 21, 2002. Each of U.S. Provisional Application Serial No. 60/303,449 and U.S. Provisional Application Serial No. 60/366,575 is incorporated herein by reference in its entirety.

### GOVERNMENT SUPPORT

This invention was made in part with government support under: Grant Nos. AI 15650, GM56685, GM59456 and A148660, all awarded by the National Institutes of Health; and Grant Nos. CONS00712 and AG95-37303-1711, each awarded by the United States Department of Agriculture. The government has certain rights to this invention.

### FIELD OF THE INVENTION

The present invention relates to the three dimensional structure of acyl-homoserine lactone synthases and to uses thereof. The present invention also relates to novel acyl-homoserine lactone synthases, nucleic acid molecules encoding such synthases, recombinant molecules and host cells, and uses thereof.

### BACKGROUND OF THE INVENTION

Bacterial quorum-sensing systems permit bacteria to sense their cell density and to initiate an altered pattern of gene expression after a sufficient quorum of cells has accumulated (Albus et al., 1977, J Bacteriol 179:3928-3935; Fuqua et al., 1999, In Cell-Cell Communication in Bacteria., G. Dunny, and S. C. Winans, eds. (AMS Press.), pp.211-230; Sitnikov et al., 1995, Mol Microbiol 17:801-812). Quorum sensing regulates the formation of bacterial biofilms that are associated with a wide variety of chronic infections caused by gram-negative opportunistic bacteria (reviewed in Davies et al., 1998, Science 280:295-298; Whitehead et al., 2001, Microbiol Rev 25:365-404). For example, the biofilm of Pseudomonas aeruginosa is made of sessile bacterial colonies encased in polysaccharide matrices that are resistant to antimicrobials and host immune cells. The biofilms severely complicate the treatment of persistently infected cystic fibrosis patients and immune-compromised individuals. Quorum sensing has also been shown to regulate gram-negative bacterial pathogenesis in plants. Pantoea stewartii, for example, is a phytopathogenic bacterium that uses quorum sensing to control the cell density-linked synthesis of an exopolysaccharide (EPS), a virulence factor in the cause of Stewart's wilt disease in maize (Beck von Bodman, 1995, J Bacteriol 177:5000-5008; Coplin et al., 1992, Mol Plant-Microbe Interact 4:81-88).

Quorum sensing in more than 30 gram-negative bacteria is mediated by lipid signaling molecules that are chemical derivatives of acyl-homoserine lactones (AHLs) (Fuqua et al., 1998, Curr Opin Microbiol 1:183-189; Swift et al., 1999, In Cell-Cell Communication in Bacteria., G. Dunny, and S. C. Winans, eds. (AMS Press.), pp. 291-313) (FIG. 1A). AHLs are synthesized by AHL synthases, enzymes also known as I-proteins, and are sensed by the response regulator family of transcription factors known as R-proteins. Intracellular accumulation of a sufficient concentration of the cell-permeable AHL generally leads to activated transcription from different promoters within the bacterial genome by induction of a transcriptionally active response regulator such as LuxR of Vibrio fischeri or LasR of P. aeruginosa (Pearson et al., 1999, J Bacteriol 181:1203-1210; Welch et al., 2000, EMBO J. 19:631-641; Zhu et al., 2001, Proc Natl Acad Sci USA 98:1507-1512). However, in several species the response regulator acts as a negative transcriptional regulator (Kanamaru et al., 2000, Mol Microbiol 38:805-816; Lewenza et al., 2001, J Bacteriol 183:2212-2218), including EsaR of P. stewartii (Beck von Bodman, 1998, Proc Natl Acad Sci USA 95:7687-7692; Minogue et al, 2002Mol. Microbiol. 44:1635-1635).

Natural and synthetic mechanisms that inhibit or misregulate quorum sensing have detrimental effects on bacterial pathogenicity. P. aeruginosa null mutants that lack the AHL synthases, LasI and RhlI, or the response regulator LasR, show a decrease in biofilm formation and attenuated pathogenicity in several in vivo infection model systems (Rumbaugh et al., 1999, Infect Immun 67:5853-5862; Tang et al., 1996, Infect Immun 64:37-43). In P. stewartii, null mutants of the AHL synthase, EsaI, are unable to produce detectable levels of EPS, and are avirulent. In contrast, mutants lacking the EsaR response regulator have a hypermucoid phenotype and reduced pathogenicity but are also avirulent, as a result of constitutive, cell density-independent, EPS synthesis (Beck von Bodman, 1998, Proc Natl Acad Sci USA 95:7687-7692). AHL-specific quorum sensing is inhibited by recently discovered halogenated furanones, produced by the marine alga Delisea pulchra, which prevent microbial and metazoan colonization (Hentzer et al., 2002, Microbiol 148:87-102). Production of enzymes that destroy the AHL, such as the N-acyl-homoserine lactonase produced by Bacillus species (Dong et al., 2001, Nature 411:813-817) or the aminoacylase produced by Variovorax paradoxus (Leadbetter et al., 2000, J Bacteriol 182:6921-6926), eliminate quorum sensing and protect the respective hosts from bacterial infection. Finally, ectopic expression of AHL synthases in plant hosts blocks infection of phytobacteria that express virulence functions in an AHL quorum sensing-dependent manner (Fray et al., 1999, Nat Biotechnol 171:1017-1020; Mäe et al., 2001, Mol Plant Microbe Interact 14:1035-1042). Therefore, strategies that either inhibit quorum sensing, or cause the premature expression of target operons can provide broad-spectrum control of particular bacterial diseases in humans, animals, and plants. To develop synthetic inhibitors of quorum sensing a better understanding of AHL synthesis is required.

AHLs are produced by the AHL-synthase from the substrates S-adenosyl-L-methionine (SAM) and acylated acyl carrier protein (acyl-ACP) in a proposed ‘bi-ter’ sequentially ordered reaction (Parsek et al., 1999, Proc Natl Acad Sci USA 96:4360-4365; Val et al., 1998, J Bacteriol 180:2644-2651) (FIG. 1B). In this reaction, the acyl-chain is presented to the AHL-synthase as a thioester of the ACP phosphopantetheine prosthetic group, which results in nucleophilic attack on the 1-carbonyl carbon by the amine of SAM in the acylation reaction. Lactonization occurs by nucleophilic attack on the gamma carbon of SAM by its own carboxylate oxygen to produce the homoserine lactone product. The N-acylation reaction, involving an enzyme-acyl-SAM intermediate, is thought to occur first, because butyryl-SAM acts as both a substrate and as an inhibitor for the P. aeruginosa AHL synthase, RhlI, to produce C4-AHL (Parsek et al., 1999, Proc Natl Acad Sci USA 96:4360-4365). A unique aspect of the AHL synthesis mechanism is that the substrates adopt roles that differ quite dramatically from their normal cellular functions. SAM usually acts as a methyl donor, whereas acyl-ACPs are components of the fatty acid biosynthetic pathway, and had not been implicated in cell-cell communication until their discovery as acyl-chain donors in AHL synthesis (More et al., 1996, Science 272:1655-1658). Further, a key step in AHL synthesis is the internal lactonization of SAM, which demands an unusual cyclic conformation that favors this reaction.

AHL-synthases from different bacterial species produce AHLs that vary in acyl chain length, from C4 to C16, oxidation at the C3 position, and saturation (De Kievit et al., 2000, Infect Immun 68:4839-4849; Kuo et al., 1994, J Bacteriol 176:7558-7565) (FIG. 1A). This variability is a function of the enzyme acyl-chain specificity, and may also be influenced by the available cellular pool of acyl-ACPs (Fray et al., 1999, Nat Biotechnol 171:1017-1020; Fuqua et al., 1999, supra). More than 40 AHL synthases, similar to the archetype LuxI (Fuqua et al., 1994, J Bacteriol 176:269-275), have been characterized, and they share four blocks of conserved sequence (FIG. 2). Within these blocks, there is on average 37% identity with eight residues that are absolutely conserved. When mutated, the most conserved residues impact catalysis of the LuxI (Vibriofischeri) and RhlI AHL-synthases (Hanzelka et al., 1997, J Bacteriol 179:4882-4887; Parsek et al., 1997, Mol Microbiol 26:301-310).

An innovative approach to the development of novel antibiotics is to target the bacterial quorum-sensing regulatory system. This approach could have far reaching implications for treatment of many human pathogens that use quorum-sensing virulence regulation, such as species of Bordetella, Enterobacter, Serratia, and Yersinia. Currently there are no antibacterials that use this approach to reduce bacterial virulence and increase susceptibility to bactericidal antibiotics. The quorum-sensing system is an antibacterial target because it is not found in humans and is critical for high level bacterial virulence. Recent studies in vivo have shown that the virulence of P. aeruginosa lacking one or more genes responsible for quorum sensing is attenuated in its ability to colonize and spread within the host. Similarly, elimination of the AHL synthase in several plant pathogenic bacteria has lead to complete loss of infectivity (Beck von Bodman, 1998, Proc Natl Acad Sci USA 95:7687-7692; Whitehead et al., 2001, Microbiol Rev 25:365-404). Moreover, ectopic expression of AHL synthases in transgenic plant systems has demonstrated that when invading bacteria encounter inducing levels of AHLs their behaviors are sufficiently modulated to shift the delicate balance of host-microbe interactions in favor of disease resistance (Fray et al., 1999, Nat Biotechnol 171:1017-1020; Mae et al., 2001, Mol Plant Microbe Interact 14:1035-1042). A number of plants, including common crop plants, produce endogenous AHL compounds, and it is thought that these AHLs are the basis of varying degrees of disease resistance and susceptibility (Teplitski et al., 2000, Mol Plant-Microbe Interact 13:637-648). Certainly, the halogenated furanones produced by some marine algae have a pronounced effect on suppressing marine biofouling.

Since mechanistic, mutagenesis, and sequence analyses have revealed a great degree of similarity among the AHL-synthases, there are a number of hypotheses about functional regions and residues. However, to understand completely the mechanism and functional regions of the AHL-synthases or embellish the mechanism any further, structural information is essential.

### SUMMARY OF THE INVENTION

One embodiment of the present invention relates to a method of structure-based identification of compounds which potentially bind to an AHL synthase. The method includes the steps of: (a) obtaining atomic coordinates that define the three dimensional structure of an AHL synthase, the atomic coordinates being selected from:

(a) a structure defined by atomic coordinates of a three dimensional structure of a crystalline AHL synthase (e.g., crystalline EsaI or crystalline LasI);

(b) a structure defined by atomic coordinates selected from:

(i) atomic coordinates represented in any one of Tables 2-5;

(ii) atomic coordinates that define a three dimensional structure having an average root-mean-square deviation (RMSD) of equal to or less than about 1.7 Å over the backbone atoms in secondary structure elements of at least 50% of the residues in a three dimensional structure represented by the atomic coordinates of (1);

wherein the structure has an amino acid sequence comprising at least three of eight conserved amino acid residues corresponding to the following residues in SEQ ID NO: 1: Arg²⁴, Phe²⁸, Trp³⁴, Asp⁴⁵, Asp⁴⁸, Ag68, Glu⁹⁷, or Arg¹⁰⁰ or to the following residues in SEQ ID NO:2: Arg²³, Phe²⁷, Trp³³, Asp⁴⁴, Asp47, Arg⁷⁰, Glu¹⁰¹ or Arg¹⁰⁴; and

wherein the structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; or

(iii) atomic coordinates in any one of Tables 2-5 defining a portion of the AHL synthase, wherein the portion of the AHL synthase comprises sufficient structural information to perform step (b);

(c) a structure defined by atomic coordinates defining the three dimensional structure of EsaI molecules arranged in a crystalline manner in a space group p4₃ so as to form a unit cell having approximate dimensions of a=b=66.40, c=47.33;

(d) a structure defined by atomic coordinates defining the three dimensional structure of EsaI molecules arranged in a crystalline manner in a space group p4₃ so as to form a unit cell having approximate dimensions of a=b=66.99, c=47.01; or

(e) atomic coordinates defining the three dimensional structure of LasI molecules arranged in a crystalline manner in a space group F23, so as to form a unit cell having approximate dimensions of a=b=c=154.90 Å.

The method further includes (b) selecting candidate compounds for binding to the AHL synthase by performing structure based drug design with the structure of (a), wherein the step of selecting is performed in conjunction with computer modeling.

In one aspect, the method includes the step of (c) selecting candidate compounds of (b) that inhibit the biological activity of an AHL synthase. For example, such a selection step can include: (i) contacting the candidate compound identified in step (b) with the AHL synthase; and (ii) measuring the enzymatic activity of the AHL synthase, as compared to in the absence of the candidate compound.

In another aspect, the method further includes the step of (c) selecting candidate compounds of (b) that inhibit the binding of an AHL synthase to its substrate. For example, such a selection step can include: (i) contacting the candidate compound identified in step (b) with the AHL synthase or a fragment thereof and a corresponding substrate or an AHL-synthase binding fragment thereof under conditions in which an AHL synthase-substrate complex can form in the absence of the candidate compound; and (ii) measuring the binding of the AHL synthase or fragment thereof to the substrate or fragment thereof, wherein a candidate inhibitor compound is selected when there is a decrease in the binding of the AHL synthase or fragment thereof to the substrate or fragment thereof, as compared to in the absence of the candidate inhibitor compound. A substrate can include, but is not limited to, S-adenosyl-L-methionine (SAM), an acylated acyl carrier protein (acyl-ACP), an acylated Coenzyme A molecule, and AHL-binding fragments thereof.

In one aspect, the step of selecting comprises identifying candidate compounds for binding to the phosphopantetheine binding fold of the AHL synthase. In another aspect, the step of selecting comprises identifying candidate compounds for binding to the acyl chain binding region of the AHL synthase. In yet another aspect, the step of selecting comprises identifying candidate compounds for binding to the acyl-ACP binding site of the AHL synthase. In another aspect, the step of selecting comprises identifying candidate compounds for binding to the SAM binding site of the AHL synthase. In another aspect, the step of selecting comprises identifying candidate compounds for binding to the electrostatic cluster of the AHL synthase.

In one embodiment of this method, the AHL synthase is a EsaI, and the atomic coordinates are selected from: (i) atomic coordinates determined by X-ray diffraction of a crystalline EsaI; (ii) atomic coordinates selected from the group consisting of: (1) atomic coordinates represented in any one of Tables 2-4; (2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by the atomic coordinates of (1), wherein the structure has an amino acid sequence comprising at least three of eight conserved amino acid residues corresponding to the following residues in SEQ ID NO: 1: Arg²⁴, Phe²⁸, Trp³⁴, Asp45, Asp⁴⁸, Arg⁶⁸, Glu⁹⁷, or Arg¹⁰⁰; and wherein the structure has an amino acid sequence comprising at least three regions having detectable sequence homology with the following three regions in SEQ ID NO: 1: amino acid residues 19 through 56, amino acid residues 63-83, and amino acid residues 90-101; and (3) atomic coordinates in any one of Tables 2-4 defining a portion of the AHL synthase, wherein the portion of the AHL synthase comprises sufficient structural information to perform step (b);(iii) atomic coordinates defining the three dimensional structure of EsaI molecules arranged in a crystalline manner in a space group p4₃ so as to form a unit cell having approximate dimensions of a—b=66.40, c=47.33; (iv) atomic coordinates defining the three dimensional structure of EsaI molecules arranged in a crystalline manner in a space group p4₃ so as to form a unit cell having approximate dimensions of a=b=66.99, c=47.01. In this embodiment, the step of selecting can comprise selecting candidate compounds for binding to the electrostatic cluster of the AHL synthase comprising positions corresponding to amino acid positions S99, R68, R100, D45, and D48 of SEQ ID NO:1. In another aspect, the step of selecting comprises selecting candidate compounds for binding to the SAM binding site of the AHL synthase comprising positions corresponding to amino acid positions 19 through 56 of SEQ ID NO: 1. In another aspect, the step of selecting comprises selecting candidate compounds for binding in a region comprising the acyl chain binding site, comprising positions corresponding to amino acid positions S98, F123, M126, T140, V142, S143, M146, I149, L150, S153, W155, I157, L176 or A178 of SEQ ID NO:1. In yet another aspect, the step of selecting comprises selecting candidate compounds for binding to the acyl chain binding site, comprising positions corresponding to amino acid positions S98, M126, T140, V142, M146, or L176 of SEQ ID NO: 1.

In another embodiment of this method, the AHL synthase is LasI, and the atomic coordinates are selected from: (i) atomic coordinates determined by X-ray diffraction of a crystalline LasI; (ii) atomic coordinates selected from the group consisting of: (1) atomic coordinates represented in Table 5; (2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by the atomic coordinates of (1), wherein the structure has an amino acid sequence comprising at least three of eight conserved amino acid residues corresponding to the following residues in SEQ ID NO:2: Arg²³, Phe²⁷, Trp³³, Asp⁴⁴, Asp⁴⁷, Arg⁷⁰, Glu¹⁰¹ or Arg¹⁰⁴; and wherein the structure has an amino acid sequence comprising at least three regions having detectable sequence homology with the following three regions in SEQ ID NO:2: amino acid residues 18-55, 65-85 and 95-105; and (3) atomic coordinates in Table 5 defining a portion of the AHL synthase, wherein the portion of the AHL synthase comprises sufficient structural information to perform step (b); and (iii) atomic coordinates defining the three dimensional structure of LasI molecules arranged in a crystalline manner in a space group F23, so as to form a unit cell having approximate dimensions of a=b=c=154.90 Å. In this aspect, the step of selecting can include selecting candidate compounds for binding to the electrostatic cluster of the AHL synthase comprising positions corresponding to amino acid positions 8, 20, 23, 42, 47, 49, 53, 67, 100 or 101 of SEQ ID NO:82. In one aspect, the step of selecting comprises selecting candidate compounds for binding to the SAM binding site of the AHL synthase comprising positions corresponding to amino acid positions 26, 27, 30, 33, 66, 102, 104, 106, 114, 140, 141, 142, or 145 of SEQ ID NO:82. In another aspect, the step of selecting comprises selecting candidate compounds for binding in a region comprising the acyl chain binding site, comprising positions corresponding to amino acid positions 99, 100, 118, 122, 137, 139, 141, 145, 148, 149, 152, 154, 175, 181, 184, or 185 of SEQ ID NO:82. In another aspect, the step of selecting comprises selecting candidate compounds for binding to the ACP binding site, comprising positions corresponding to amino acid positions 147, 150, 151 or 180 of SEQ ID NO:82.

The step of selecting in this method of the present invention can be performed using any suitable technique, including but not limited to, directed drug design, random drug design, grid-based drug design, and/or computational screening of one or more databases of chemical compounds.

Yet another embodiment of the present invention relates to a method to produce an AHL synthase homologue that catalyzes the synthesis of AHL compounds having antibacterial biological activity. The method includes the steps of: (a) obtaining atomic coordinates that define the three dimensional structure of an AHL synthase as described in the method above; (b) performing computer modeling with the atomic coordinates of (a) to identify at least one site in the AHL synthase structure that is predicted to modify the biological activity of the AHL synthase; (c) producing a candidate AHL synthase homologue that is modified in the at least one site identified in (b); and (d) determining whether the candidate AHL synthase homologue of (c) catalyzes the synthesis of AHL compounds having antibacterial biological activity.

Another embodiment of the present invention relates to a method to produce an AHL synthase homologue with modified biological activity as compared to a natural AHL synthase. The method includes the steps of: (a) obtaining atomic coordinates that define the three dimensional structure of an AHL synthase as described in the method above; (b) using computer modeling of the atomic coordinates in (a) to identify at least one site in the AHL synthase structure that is predicted to contribute to the biological activity of the AHL synthase; and (c) modifying the at least one site in an AHL synthase protein to produce an AHL synthase homologue which is predicted to have modified biological activity as compared to a natural AHL synthase. In one aspect, the step of modifying in (c) comprises using computer modeling to produce a structure of an AHL synthase homologue on a computer. In another aspect, the step of modifying in (c) comprises making at least one modification in the amino acid sequence of the AHL synthase protein selected from the group consisting of an insertion, a deletion, a substitution and a derivatization of an amino acid residue in the amino acid sequence. In another aspect, the method further comprises a step of determining whether the AHL synthase homologue has modified AHL synthase biological activity.

Yet another embodiment of the present invention relates to a method to construct a three dimensional model of an AHL synthase. The method includes: (a) obtaining atomic coordinates that define the three dimensional structure of a first AHL synthase as described in the methods above; and (b) performing computer modeling with the atomic coordinates of (a) and an amino acid sequence of a second AHL synthase to construct a model of a three dimensional structure of the second AHL synthase. In one aspect, step (b) is performed using molecular replacement. In another aspect, the second AHL synthase is a naturally occurring AHL synthase or alternatively, the second AHL synthase is a homologue of the first AHL synthase. In one aspect, the second AHL synthase is from a microorganism listed in Table 1. In one aspect, the second AHL synthase is from a mycobacterium, including but not limited to, Mycobacterium tuberculosis.

Another embodiment relates to a crystal comprising an AHL synthase, wherein the crystal effectively diffracts X-rays for the determination of the atomic coordinates of the AHL synthase to a resolution of greater than 3.2 Å, and wherein the crystal has a space group p⁴₃ so as to form a unit cell having approximate dimensions of a=b=66.40, c=47.33.

Yet another embodiment relates to a crystal comprising an AHL synthase, wherein the crystal effectively diffracts X-rays for the determination of the atomic coordinates of the AHL synthase to a resolution of greater than 3.2 Å, and wherein the crystal has a space group p4₃ so as to form a unit cell having approximate dimensions of a=b=66.99, c=47.01.

Yet another embodiment relates to a crystal comprising an AHL synthase, wherein the crystal effectively diffracts X-rays for the determination of the atomic coordinates of the AHL synthase to a resolution of greater than 3.2 Å, and wherein the crystal has a space group F23, so as to form a unit cell having approximate dimensions of a=b=c=154.90 A.

Another embodiment of the present invention relates to a therapeutic composition comprising a compound that inhibits the biological activity of an AHL synthase. The compound is identified by the method comprising: (a) obtaining atomic coordinates that define the three dimensional structure of an AHL synthase as described in the methods above; (b) selecting candidate compounds for binding to the AHL synthase by performing structure based drug design with the structure of (a), wherein the step of selecting is performed in conjunction with computer modeling; (c) synthesizing the candidate compound selected in (b); and (d) further selecting candidate compounds that inhibit the biological activity of the AHL synthase. One aspect of the invention relates to a method to treat a disease or condition that can be regulated by modifying the biological activity of an AHL synthase or a compound produced by the enzymatic activity of the synthase, comprising administering to an organism with such a disease or condition the therapeutic composition described above. If desired, the method can further include administering to the organism an antibacterial agent.

Another embodiment of the present invention relates to a transgenic plant or part of a plant comprising one or more cells that recombinantly express a protein. In one aspect, the protein is a protein compound identified by the method of structure based drug design described above. In another aspect, the protein is an AHL synthase homologue that is identified using a computer modeling method described above.

Another embodiment of the present invention relates to an isolated protein comprising a mutant AHL synthase, wherein the protein comprises an amino acid sequence that differs from the amino acid sequence of a naturally occurring AHL synthase by at least one amino acid modification that results in a mutant AHL synthase that catalyzes the production of a different AHL product as compared to the naturally occurring AHL synthase. In one embodiment, the protein comprises an amino acid sequence that differs from the amino acid sequence of a naturally occurring AHL synthase by at least one amino acid modification in the acyl chain binding region of the AHL synthase. In another aspect, the protein comprises a mutation in an amino acid residue corresponding to Thr¹⁴⁰ in SEQ ID NO: 1. In another aspect, the protein comprises a mutation in an amino acid residue corresponding to Ser⁹⁹ of SEQ ID NO: 1. Another aspect relates to a transgenic plant or part of a plant comprising one or more cells that recombinantly express a nucleic acid sequence encoding a such a mutant AHL synthase.

Another embodiment of the present invention relates to an isolated protein comprising a mutant EsaI protein, wherein the protein comprises an amino acid sequence that differs from SEQ ID NO: 1 by at least one modification including at least one amino acid substitution selected from the group consisting of: a non-arginine amino acid residue at position 24, a non-phenyalanine amino acid residue at position 28, a non-tryptophan amino acid residue at position 34, a non-aspartate amino acid residue at position 45, a non-aspartate amino acid residue at position 48, a non-arginine amino acid residue at position 68, a non-glutamate amino acid residue at position 97, a non-serine amino acid residue at position 99, a non-arginine amino acid residue at position 100; and a non-threonine amino acid residue at position 140, wherein the mutant EsaI protein has modified biological activity as compared to a wild-type EsaI protein. In one aspect, the protein comprises an amino acid sequence that differs from SEQ ID NO:1 by at least one modification including a substitution of a non-threonine amino acid residue at position 140. In another aspect, the protein comprises an amino acid sequence that differs from SEQ ID NO: 1 by at least one modification including a substitution of a non-serine amino acid residue at position 99. In another aspect, the protein comprises an amino acid sequence that differs from SEQ ID NO: 1 by an amino acid substitution selected from the group consisting of: an asparagine substituted for the aspartate at position 45, a glutamine substituted for the glutamate at position 97, an alanine substituted for the serine at position 99; a valine substituted for the threonine at position 140; and an alanine substituted for the threonine at position 140.

Yet another embodiment of the present invention relates to an isolated AHL synthase comprising an amino acid sequence selected from: (a) an amino acid sequence that is at least about 70% identical to an amino acid sequence selected from any of SEQ ID NOs:67 or SEQ ID NOs:83-100, wherein the amino acid sequence has AHL synthase activity; and (b) a fragment of an amino acid sequence of (a), wherein the fragment has AHL synthase activity. In a preferred embodiment, the amino acid sequence is at least about 80% identical, and more preferably at least about 90% identical, to an amino acid sequence selected from any of SEQ ID NOs:67 or SEQ ID NOs:83-100, wherein the amino acid sequence has AHL synthase activity. In another embodiment, the amino acid sequence is less than 100% identical, and in another embodiment less than about 98% identical to an amino acid sequence selected from any of SEQ ID NOs:67 or SEQ ID NOs:83-100, wherein the amino acid sequence has AHL synthase activity. In one aspect, the AHL synthase is from a mycobacterium, including but not limited to, Mycobacterium tuberculosis, Mycobacterium avium, Mycobacterium bovis, and Mycobacterium leprae.

Another embodiment of the present invention relates to an isolated nucleic acid molecule comprising a nucleic acid sequence selected from the group consisting of: (a) a nucleic acid sequence that encodes an amino acid sequence that is at least about 70% identical and less than 100% identical to an amino acid sequence selected from any of SEQ ID NOs:67 or SEQ ID NOs:83-100, wherein the amino acid sequence has AHL synthase activity; (b) a nucleic acid sequence encoding a fragment of the amino acid sequence of (a), wherein the fragment has AHL synthase activity; (c) a nucleic acid sequence that is a probe or primer that hybridizes under high stringency conditions to a nucleic acid sequence of (a) or (b); and (d) a nucleic acid sequence that is a complement of any of the nucleic acid sequences of (a)-(c). In one embodiment, the nucleic acid sequence encodes an amino acid sequence that is at least about 80% identical and less than 100% identical to an amino acid sequence selected from any of SEQ ID NOs:67 or SEQ ID NOs:83-100, wherein the amino acid sequence has AHL synthase activity. In another embodiment, the nucleic acid sequence encodes an amino acid sequence that is at least about 90% identical and less than 100% identical to an amino acid sequence selected from any of SEQ ID NOs:67 or SEQ ID NOs:83-100, wherein the amino acid sequence has AHL synthase activity.

Another aspect of the invention relates to a recombinant nucleic acid molecule comprising a nucleic acid molecule described above that is operatively linked to at least one transcription control sequence. Another aspect of the invention relates to a recombinant host cell transformed with a recombinant nucleic acid molecule described above. The host cell can include a prokaryotic cell or a eukaryotic cell.

Another embodiment of the present invention relates to an isolated AHL synthase comprising an amino acid sequence selected from the group consisting of: (a) an amino acid sequence that is at least about 30% identical to SEQ ID NO:67, wherein the amino acid sequence comprises at least three amino acid residues corresponding to amino acid residues of SEQ ID NO:67 selected from: Arg⁹, Phe¹³, Phe¹⁹, Asp³², Asp³⁵, Arg⁵⁶, Glu⁸⁹ and Arg⁹², and wherein the amino acid sequence has AHL synthase activity; and (b) a fragment of an amino acid sequence of (a), wherein the fragment has AHL synthase activity.

Yet another embodiment of the present invention relates to a method of identifying a compound that regulates quorum sensing signal generation. The method includes the steps of: (a) contacting an AHL synthase or biologically active fragment thereof with a putative regulatory compound, wherein the AHL synthase comprises an amino acid sequence that is at least about 70% identical to an amino acid sequence selected from any of SEQ ID NOs:67 or SEQ ID NOs:83-100, or a biologically active fragment thereof, wherein the amino acid sequence has AHL synthase activity; (b) detecting whether the putative regulatory compound increases or decreases a biological activity of the AHL synthase as compared to in the absence of contact with the compound. Compounds that increases or decreases activity of the AHL synthase, as compared to in the absence of the compound, indicates that the putative regulatory compound is a regulator of the AHL synthase. Biological activity can include, but is not limited to, the binding of the AHL synthase to a substrate, AHL enzymatic activity, synthesis of an AHL, quorum sensing signal generation in a population of microorganisms expressing the AHL synthase, and change in production of gene products dependent on the transcription factors that bind the AHL.

Another embodiment of the present invention relates to a method to inhibit quorum sensing signal generation in a population of microbial cells, comprising contacting a population of microbial cells that express an AHL synthase with an antagonist of the AHL synthase, wherein the antagonist decreases the biological activity of the AHL synthase, and wherein the AHL synthase comprises an amino acid sequence that is at least about 70% identical to an amino acid sequence selected from any of SEQ ID NOs:67 or SEQ ID NOs:83-100. In one aspect, the population of microbial cells infects a plant. The plant can be transgenic for the expression of the antagonist of the AHL synthase. In another aspect, the population of microbial cells infects an animal.

### BRIEF DESCRIPTION OF THE DRAWINGS

FIG. 1A is a schematic drawing showing that the structures of three AHLs show variation in acyl-chain length and degree of oxidation at the acyl-chain C3 position.

FIG. 1B is a schematic diagram illustrating the general features of the AHL synthesis reaction. Two substrates, acyl-ACP and SAM bind to the enzyme. After the acylation and lactonization reactions, the product AHL and byproducts holo-ACP and 5′-methylthioadenosine are released.

FIG. 2 is an alignment showing the sequence and topology of the AHL synthase family as compared to the GCN5-related N-acetyltransferases (EsaI=SEQ ID NO: 1; LuxI=SEQ ID NO:3; LasI=SEQ ID NO:2; RhlI=SEQ ID NO:29; tGCN5=SEQ ID NO:77; AANAT=SEQ ID NO:78; AAC-6′=SEQ ID NO:79).

FIG. 3A is a digitized image of a stereoview of a simulated annealing composite omit map (2Fo-Fc) contoured at 1σ illustrates the environment of four rhenium ions in the protein.

FIG. 3B is a digitized image of a GRASP (Nicholls et al., 1993, Biophysical J64:A166) surface representation of EsaI in stereoview shaded according to the calculated electrostatic potential with charged surfaces shaded in grays; the five positively identified perrhanate ions, based on their anomalous signal by SOLVE, are shown as spheres.

FIG. 4A is a ribbon diagram which indicates the N- to C-terminal positions of residues within the EsaI sequence.

FIG. 4B is a digitized image of a surface rendering of EsaI showing absolutely conserved residues in darkest gray, homologous residues in lightest shades of gray, and non-homologous residues in medium gray.

FIG. 4C is a digitized image of the electrostatic cluster of conserved residues.

FIG. 5A is a stereodiagram of acyl-phosphopantetheine modeled into the EsaI active-site cavity viewed as in FIG. 3A (generated using GRASP (Nicholls et al., 1993, Biophysical J 64:A166) and Photoshop (Adobe)).

FIG. 5B is a digitized image of the EsaI structure, showing the acylation cleft of EsaI and relevant residues, the modeled phosphopantheteine, and the well-ordered water molecules observed in the native structure that lie along P4, shown as spheres.

FIG. 5C is a schematic diagram showing that the proposed N-acylation reaction is catalyzed via nucleophilic attack on the 1-carbonyl of acyl-ACP by the free amine electrons of SAM, after proton abstraction by a water molecule stabilized by Glu⁹⁷ or Ser⁹⁹.

FIG. 6 is an alignment showing the sequence and topology of the AHL synthases: EsaI (SEQ ID NO: 1), LuxI (SEQ ID NO:3), LasI (SEQ ID NO:2), RhlI (SEQ ID NO:29), YpeI (SEQ ID NO:63) and the putative AHL synthase MtuI (SEQ ID NO:67), as compared to the GCN5-related N-acetyltransferases (tGCN5=SEQ ID NO:77; AANAT=SEQ ID NO:78; AAC-6′=SEQ ID NO:79).

FIG. 7 is a digitized image ribbon diagram of LasI, which indicates the N- to C-terminal positions of residues within the LasI sequence, and also shows well-ordered water molecules and ions.

FIG. 8 is a digitized image of a SPOCK (Jon A. Christopher) surface representation of LasI shaded according to the calculated electrostatic potential.

FIG. 9 is a digitized image of a ribbon diagram showing a superposition of LasI (in light gray) and EsaI (in darker gray).

### DETAILED DESCRIPTION OF THE INVENTION

The present invention relates to the determination of the structure of the active site of enzymes involved in the quorum sensing system of microorganisms, known as acylhomoserine lactone (AHL) synthases, and to the use of such structures to develop inhibitors and lead compounds for drug development in the area of therapeutic agents against pathogenic microorganisms. The present invention also relates to the discovery of new AHL synthases that were not previously recognized to be AHL synthases, to structural models of and to the use of such synthases to identify and develop drugs and lead compounds in the area of antimicrobial therapeutics.

More particularly, the present inventors have identified structure of the catalytic site surface of the acylhomoserine lactone (AHL) synthases, EsaI and LasI, as well as the residues that are important for catalysis. In addition, the present inventors propose a mechanism for acylation. Using this knowledge, one can design structure-based inhibitors of the enzymes and use these structures to model other AHL synthases that are predicted to have similar structures.

The present inventors have also identified the residues of EsaI that are important for specific AHL synthase production, which is demonstrated by mutagenesis and functional studies. This has applications for designing novel AHL synthases to produce altered AHL compounds as antibacterial agents and for commercial production purposes. These novel synthases could be put into transgenic animals, plants or used in gene therapy, for example, to produce altered bacterial behavior.

The present inventors have speculated that the structure of the AHL synthase enzymes disclosed herein shows similarity to non-Lux-I type AHL synthases (e.g., AinS, LuxM, VanM). The regions of AinS, LuxM and VanM that correspond are:

AinS: SILDKTKVCEAIRLTISGSKSKA (SEQ ID NO:74)

LuxM: LSDTQAVCEVLRLTVSGNAQQK (SEQ ID NO:75)

VanM: LTGTQAVCEVLRLTVSGNAQQK (SEQ ID NO:76)

The data presented herein suggests that the non-Lux-I type AHL synthases may use a similar mechanism based on sequence homology to Lux-I type AHL synthase block 3 alignment. However, the non-LuxI type AHL synthases do not meet the additional more stringent criteria that the present inventors have identified for classical AHL synthases, which include having at least three of the eight amino acid residues that are absolutely conserved in the synthases described by the present invention, and having at least three and preferably the first three, of the four blocks of sequence homology that have been identified for these synthases (described in detail below). Therefore, for the purposes of this invention, the non-LuxI type AHL synthases are not considered to be structural homologues of the AHL synthase structures of the present invention.

Therefore, the present invention relates to the discovery of the three-dimensional structure of the acylhomoserine lactone (AHL) synthase—EsaI, to the discovery of the three-dimensional structure of LasI, to crystalline EsaI, to crystalline LasI, to models of AHL synthase three-dimensional structures (including EsaI and LasI structures), to the surface residues of AHL-synthases that may be targeted for inhibition or alteration of function, to a method of structure based drug design using such structures, to the design of novel AHL synthases using such structures, to the compounds identified by structure based drug design using such structures and to the use of such compounds in therapeutic compositions and methods. The present invention also relates to the discovery of a class of proteins from mycobacterium which are believed to be AHL synthases and which are predicted to have a similar structure to the AHL synthases described herein. Preferably, the structures disclosed herein are used to design and/or identify novel antibacterial agents or anti-mycobacterial agents which can be used in various systems, including in gene therapy and in the production of transgenic plants and other organisms.

The present inventors have determined the structure of the AHL synthase, EsaI, by X-ray crystallography. The structure, at a resolution of 1.8 Å, provides the basis for the interpretation of past mutagenesis and biochemical results and an understanding of the N-acylation step in AHL synthesis. A model of the enzyme-phosphopantetheine complex shows novel interactions important for specificity of AHL synthesis through substrate recognition. The activity and specificity of structure-based mutants, determined from complementary in vivo biological reporter assays, verify the proposed roles of several residues involved in catalysis or enzyme-substrate specificity. Further, the present inventors demonstrate herein the ability to alter the product distribution of the AHL synthase by making a single key mutation. This structure reveals the roles of many conserved residues and provides a mechanistic basis for the first step in AHL synthesis.

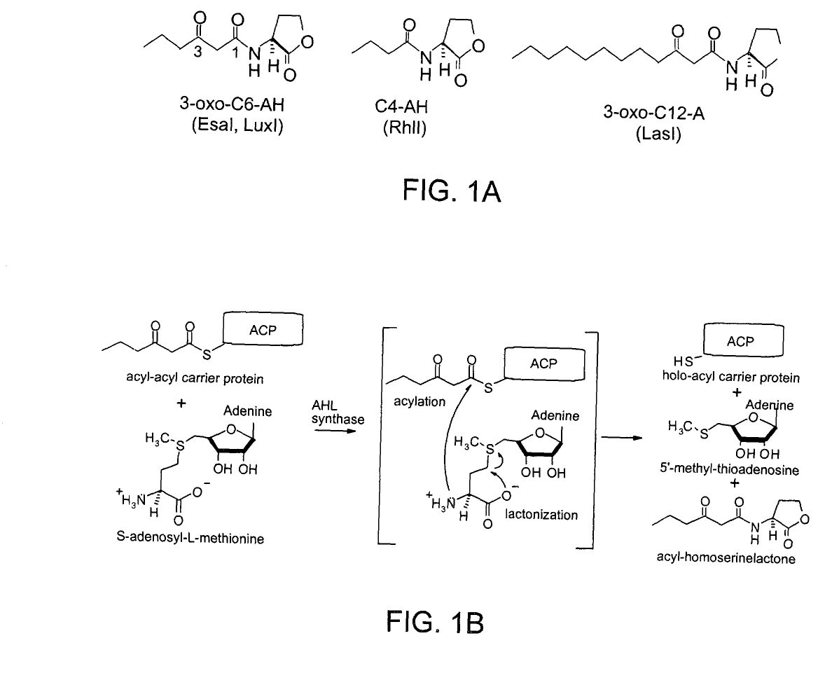

EsaI produces primarily a 3-oxo-hexanoyl-homoserine lactone, which contributes to the quorum-sensing regulation of pathogenicity in Pantoea stewartii subsp. stewartii (Beck von Bodman et al., 1995, J Bacteriol 177:5000-5008). EsaI is representative of the AHL synthase family of proteins, having 28% identity (42% homology) and 23% identity (43% homology) with the P. aeruginosa AHL synthases LasI and RhlI respectively, and preferentially produces an AHL of intermediate length (FIG. 1A).

The EsaI structure reveals that the core catalytic fold of the AHL synthase family has features essential for phosphopantetheine binding and N-acylation that are similar to the GNAT family of N-acetyltransferases. The modeling study and GNAT structural analysis suggests that the reaction mechanism of the first step in AHL-mediated quorum sensing signal generation, the N-acylation reaction of SAM, is also likely to include a similar type of amine proton abstraction by a catalytic base. In addition, variable residues in the C-terminal half of the protein, and the presence or absence of a Ser/Thr at position 140, constitute the basis for the acyl-chain specificity. Other enzymes in gram negative bacteria that synthesize lipid communication signals, such as the LuxM-type AHL synthases, for example, LuxM, AinS, and VanM (Hanzelka et al., 1999, J Bacteriol 181:5766-5770; Hanzelka et al., 1997, J Bacteriol 179:4882-4887; Parsek et al., 2000, Proc Natl Acad Sci USA 97:8789-8793; Parsek et al., 1997, Mol Microbiol 26:301-310), also appear to share some sequence homology with EsaI, particularly in the conserved block 3 catalytic region. Not surprisingly, a novel quorum-sensing system, mediated by the LuxS and LuxP gene products, which synthesizes and responds to the AI-2 molecule (Chen et al. 2002, Nature 415:545-549; Lewis et al., 2001, Structure 9:527-537), is distinct chemically and structurally from the AHL-mediated system described here.

The present inventors have also determined the three-dimensional structure of a second AHL synthase, LasI from P. aeruginosa, also by X-ray crystallography, and have further identified target sites on the LasI molecule for drug design and lead compound development.

Finally, the present inventors have identified a putative protein from Mycobacterium tuberculosis and related proteins from other mycobacterial species which are believed to be AHL synthases and which are predicted to have a similar structure to the AHL synthases described herein.

Understanding the molecular mechanisms underlying quorum sensing at the atomic level will greatly enhance the ability to design new inhibitory compounds to fight pathogenic bacteria of many different species. As discussed above, recent studies in vivo have shown that the regulation of the AHL-mediated quorum sensing system in various bacteria can lead to an attenuation of the pathogenicity of the bacterium or a complete loss of infectivity (see Background section). These examples all underscore the potential to control a wide range of bacterial diseases and biofilm formation in industrial, medical, and ecological settings. Therefore, the AHL synthase structures presented herein set the stage for future structure-based approaches to develop novel inhibitors to fight persistent biofilm-mediated infections (Finch et al., 1998, J Antimicrob Chemo 42:569-571) and biofilm-based ecological problems specifically due to gram negative bacteria (Dalton et al., 1998, Curr Opin Biotechnol 9:252-255).

According to the present invention, the EsaI protein is an AHL synthase from Pantoea stewartii, also known as Erwinia stewarti, which is characterized by the amino acid sequence represented by SEQ ID NO: 1. SEQ ID NO: 1 represents the full-length EsaI protein sequence. Amino acid positions for EsaI described herein are made with reference to SEQ ID NO: 1. The crystal structure of the EsaI protein described herein comprises amino acid positions 2 to 210 of SEQ ID NO: 1. The EsaI protein used for crystallization included an N-terminal His₆ tag, facilitating isolation and purification using nickel-agarose affinity chromatography.

According to the present invention, the LasI protein is an AHL synthase from Pseudomonas aeruginosa, the native enzyme of which is characterized by the amino acid sequence represented by SEQ ID NO:2. SEQ ID NO:2 represents the full-length native LasI sequence. The crystal structure of the LasI protein described herein is of an enzymatically active mutant of the LasI protein, called LasIΔG and having the amino acid sequence represented by SEQ ID NO:82. SEQ ID NO:82 differs from SEQ ID NO:2 by a substitution of a single Gly residue for the Thr-Pro-Glu-Ala at positions 61-64 of SEQ ID NO:2. Amino acid positions described for the LasI structure described herein are made with reference to SEQ ID NO:82. The construct used to crystallize the LasI mutant included the remains of a thrombin cleaved His₆ Tag from the pViet vector.

Other AHL synthases are known in the art or have been identified by the present inventors as putative AHL synthases. A list of these synthases, the organisms from which they are derived, the amino acid sequences encoding them and the public database accession numbers for the sequences is provided in Table 1A and Table 1B (see Table 1B in text below). Such synthases are believed, without being bound by theory, to have structures similar to those described herein for EsaI and LasI. Therefore, one can use the structures for either of EsaI or LasI to model the three dimensional structures of any of the proteins in Table 1A and Table 1B and use such structures in a method of computer-assisted drug design as described in detail herein. The use of models of any of the proteins in Table 1A or Table 1B is explicitly contemplated by the present invention. In addition, with the successful crystallization of two AHL synthases as described herein, one of skill in the art can apply this experimental information to crystallize and determine the structure of any of the proteins in Table 1 A or Table 1B.

TABLE 1A

| Organism | Protein | Accession No. | |
|---|---|---|---|
| Erwinia stewarti | EsaI | 1706699 | SEQ ID NO:1 |
| Pseudomonas aeruginosa | LasI | 462480 | SEQ ID NO:2 |
| Vibrio fiseheri | LuxI | 126531 | SEQ ID NO:3 |
| Pseudomonas aerugionsa | RhlI | 1117919 | SEQ ID NO:4 |
| Aeromonas hydrophila | AhyI | 4376116 | SEQ ID NO:5 |
| Aeromonas salmonicida | AsaI | 2497765 | SEQ ID NO:6 |
| Burkholderia ambifaria | BafI | 13508494 | SEQ ID NO:7 |
| Burkholderia cepacia | BceI | 4103043 | SEQ ID NO:8 |
| Burkholderia cepacia | CepI | 12620887 | SEQ ID NO:9 |
| Burkholderia cepacia | BviI | 13625779 | SEQ ID NO:10 |
| Burkholderia cepacia | CpeI | 12620891 | SEQ ID NO:11 |
| Burkholderia cepacia | CepI | 12620897 | SEQ ID NO:12 |
| Burkholderia multivorans | CepI | 12620889 | SEQ ID NO:13 |
| Burkholderia multivorans | CepI | 126208917 | SEQ ID NO:14 |
| Burkholderia multivorans | CepI | 12620915 | SEQ ID NO:15 |
| Burkholderia multivorans | CepI | 12620913 | SEQ ID NO:16 |
| Burkholderia multivorans | CepI | 12620911 | SEQ ID NO:17 |
| Burkholderia vietnamiensis | CepI | 12620895 | SEQ ID NO:18 |
| Burkholderia stabilis | CepI | 12620893 | SEQ ID NO:19 |
| Erwinia carotovora | CarI | 461694 | SEQ ID NO:20 |
| Erwinia carotovora | CarI | 628640 | SEQ ID NO:21 |
| Erwinia carotovora subsp. | EcbI | 2367438 | SEQ ID NO:22 |
| betavasculorum | | | |
| Erwinia carotovora | ExpI | 462042 | SEQ ID NO:23 |
| Erwinia carotovora | HslI | 685172 | SEQ ID NO:24 |
| Erwinia chrysanthemi | ExpI | 2497767 | SEQ ID NO:25 |
| Erwinia chrysanthemi | EchI | 2497766 | SEQ ID NO:26 |
| Pantoea agglomerans | EagI | 461982 | SEQ ID NO:27 |
| Enterobacter agglomerans | EagI | 628632 | SEQ ID NO:28 |
| Pseudomonas aeruginosa | RhlI | 12230962 | SEQ ID NO:29 |
| Pseudomonas aeruginosa | RhlI | 511478 | SEQ ID NO:30 |
| Pseudomonas aeruginosa | RhlI | 7465475 | SEQ ID NO:31 |
| Pseudomonas aeruginosa | VsmI | 695154 | SEQ ID NO:32 |
| Pseudomonas corrugata | PcoI | 11066345 | SEQ ID NO:33 |
| Pseudomonas aureofaciens | PhzI | 2497768 | SEQ ID NO:34 |
| Pseudomonas fluorescens | AfmI | 7385147 | SEQ ID NO:35 |
| Pseudomonas fluorescens | PhzI | 2497769 | SEQ ID NO:36 |
| Pseudomonas fluorescens | MupI | 13507197 | SEQ ID NO:37 |
| Pseudomonas fluorescens | RhlI | 7385150 | SEQ ID NO:38 |
| Pseudomonas chlororaphis | PhzI | 6572976 | SEQ ID NO:39 |
| Pseudomonas syringae tabaci | PsyI | 1709884 | SEQ ID NO:40 |
| Pseudomonas syringae | AhlI | 3264776 | SEQ ID NO:41 |
| syringae | | | |
| Pseudomonas syringae | PsmI | 13182978 | SEQ ID NO:42 |
| maculicola | | | |
| Ralstonia solanacearum | SolI | 2444468 | SEQ ID NO:43 |
| Rhizobium etli | RetI or RaiI | 2897877 | SEQ ID NO:44 |
| Rhizobium leguminosarum | CinI | 9622951 | SEQ ID NO:45 |
| Rhodobacter spaeroides | CerI | 2360977 | SEQ ID NO:46 |
| Serratia sp. | SmaI | 8217386 | SEQ ID NO:47 |
| Serratia liquefaciens | SwrI | 1711621 | SEQ ID NO:48 |
| Agrobacterium tumefaciens | TraI | 464916 | SEQ ID NO:49 |
| Agrobacterium tumefaciens | TraI | 2982704 | SEQ ID NO:50 |
| Plasmid pTiC58 | TraI | 464915 | SEQ ID NO:51 |
| Rhizobium sp. | TraI | 2497770 | SEQ ID NO:52 |
| Rhizobium rhizogenes | TraI | 10954777 | SEQ ID NO:53 |
| Mesorhizobium loti | TraI | 13475097 | SEQ ID NO:54 |
| Mesorhizobium loti | TraI | 13475341 | SEQ ID NO:55 |
| Mesorhizobium loti | mlr9546 | 13488405 | SEQ ID NO:56 |
| Mesorhizobium loti | mlr5638 | 13474693 | SEQ ID NO:57 |
| Vibrio fischeri | LuxI | 297490 | SEQ ID NO:58 |
| Vibrio fischeri | LuxJ | 462555 | SEQ ID NO:59 |
| Vibrio (listonella) | VanI | 1568659 | SEQ ID NO:60 |
| anguillarum | | | |
| Yersinia enterocolitica | YenI | 541225 | SEQ ID NO:61 |
| Yersinia enterocolitica | YenI | 1723595 | SEQ ID NO:62 |
| Yersinia pestis | YpeI | 6648673 | SEQ ID NO:63 |
| Yersinia pseudotuberculosis | YtbI | 3388090 | SEQ ID NO:64 |
| Yersinia pseudotuberculosis | YpsI | 5162958 | SEQ ID NO:65 |
| Yersinia ruckeri | YukI | 3388086 | SEQ ID NO:66 |
| Mycobacterium tuberculosis | MtuI | 2791625 | SEQ ID NO:67 |
| Mycobacterium tuberculosis | N/A | 13882904 | SEQ ID NO:68 |
| Streptomyces coelicolor | N/A | 6580635 | SEQ ID NO:69 |
| Mycobacterium avium | MavI | | SEQ ID NO:70 |
| Mycobacterium bovis | Mbov | | SEQ ID NO:71 |
| Vibrio cholerae | ElaA | 11354888 | SEQ ID NO:72 |
| Xylella fastidiosa | Xfa | 11345985 | SEQ ID NO:73 |

According to the present invention, general reference to an AHL synthase is reference to a protein that, at a minimum, contains any biologically active portion (e.g., enzymatically active portion or a portion that at least binds to a given substrate) of an AHL synthase, and includes full-length AHL synthases, biologically active fragments of AHL synthases, AHL synthase fusion proteins, or any homologue of a naturally occurring AHL synthase, as described in detail below. A homologue of an AHL synthase includes proteins which differ from a naturally occurring AHL synthase in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). Preferably, an AHL synthase homologue has an amino acid sequence that is at least about 30% identical to the amino acid sequence of a naturally occurring AHL synthase (e.g., any of SEQ ID NO: 1 to SEQ ID NO:73), and more preferably, at least about 35%, and more preferably, at least about 40%, and more preferably, at least about 45%, and more preferably, at least about 50%, and more preferably, at least about 55%, and more preferably, at least about 60%, and more preferably, at least about 65%, and more preferably, at least about 75%, and more preferably, at least about 75%, and more preferably, at least about 80%, and more preferably, at least about 85%, and more preferably, at least about 90%, and more preferably, at least about 95% identical to the amino acid sequence of a naturally occurring AHL synthase.

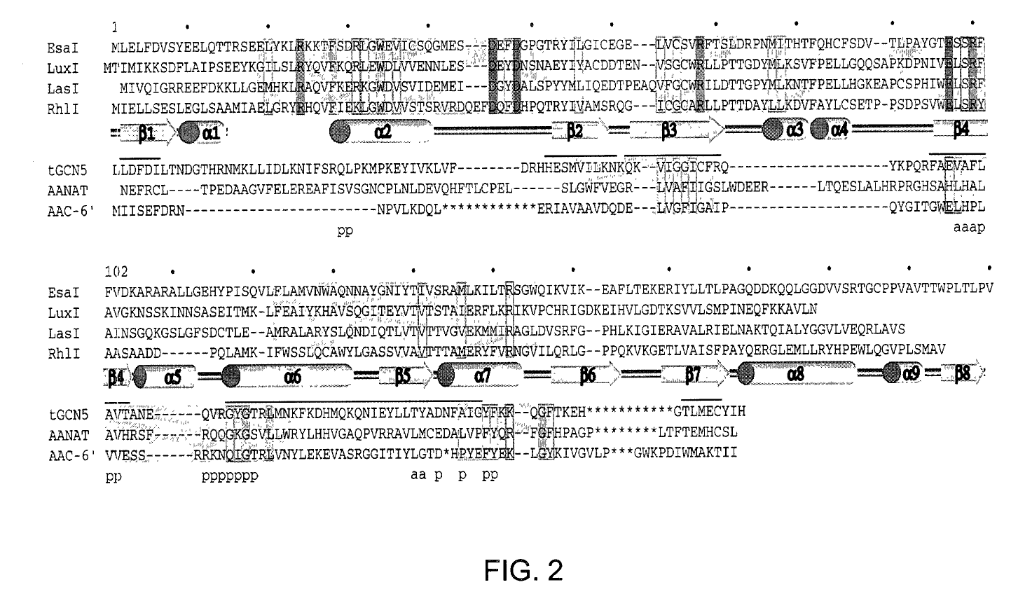

As discussed above, more than 40 AHL synthases, similar to the archetype LuxI (Fuqua et al., 1994, J Bacteriol 176:269-275), have been characterized, and they share four blocks of conserved sequence (FIG. 2). Within these blocks, there is on average 37% identity with eight residues that are absolutely conserved. Therefore, in another embodiment, preferably, an AHL synthase homologue has at least a detectable homology with an amino acid sequence that corresponds to at least one, and preferably two, and more preferably three, and even more preferably four, of these conserved blocks of sequences. In one embodiment, an AHL synthase homologue has an amino acid sequence that is at least about 20% identical to an amino acid sequence that corresponds to at least one, and preferably two, and more preferably three, and even more preferably four, of these conserved blocks of sequences. More preferably, an AHL synthase homologue has an amino acid sequence that is at least about 25% identical, and more preferably at least about 30% identical, and more preferably at least about 35% identical, and more preferably at least about 40% identical, and more preferably at least about 45% identical, and more preferably at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical, and more preferably at least about 90% identical, and more preferably at least about 95% identical, to an amino acid sequence that corresponds to at least one, and preferably two, and more preferably three, and even more preferably four, of these conserved blocks of sequences. By way of example, in EsaI (SEQ ID NO: 1), these four blocks of conserved sequences correspond to positions 19-56 (block one), 63-83 (block two), 90-101 (block three), and 123-155 (block four). In LasI (SEQ ID NO:2), these four blocks of conserved sequences correspond to positions 18-55 (block one), 65-85 (block two), 95-105 (block three), and 125-157 (block four). One of skill in the art can readily determine whether a given sequence aligns with another sequence, as well as identify conserved regions of sequence identity or homology within sequences, by using any of a number of software programs that are publicly available. For example, one can use BLOCKS (GIBBS) and MAST (Henikoffet al., 1995, Gene, 163, 17-26; Henikoffet al., 1994, Genomics, 19, 97-107), typically using standard manufacturer defaults.

Preferably, an AHL synthase homologue has an amino acid sequence comprising at least three and more preferably four, and more preferably five, and more preferably six, and more preferably seven, and even more preferably eight, out of eight absolutely conserved amino acid residues in LuxI type AHL synthases. In EsaI (SEQ ID NO: 1), these residues correspond to amino acid positions Arg²⁴, Phe²⁸, Trp³⁴, Asp⁴⁵, Asp⁴⁸, Arg⁶⁸, Glu⁹⁷, or Arg¹⁰⁰. In LasI (SEQ ID NO:2), these residues correspond to the amino acid positions: Arg²³, Phe²⁷, Trp³³, Asp⁴⁴, Asp47, Arg⁷⁰, Glu¹⁰¹ or Arg¹⁰⁴. One of skill in the art can readily determine whether a given sequence has conserved residues that correspond to a given sequence by using any of a number of software programs that are publicly available, including the programs BLOCKS (GIBBS) and MAST described above (using standard manufacturer defaults).

Preferred three-dimensional structural homologues of an AHL synthase are described in detail below. In one embodiment, an AHL synthase homologue has the ability to bind to a substrate of an AHL synthase (e.g., S-adenosyl-L-methionine (SAM), acylated acyl carrier protein (acyl-ACP), an acylated Coenzyme A molecule, or AHL synthase-binding portions thereof). Such homologues include fragments or mutants of a full length AHL synthase and can be referred to herein as a substrate-binding fragment or protein. In one embodiment, an AHL synthase homologue has a biological activity of a naturally occurring AHL synthase.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein. As used herein, a protein that has “AHL synthase biological activity” or that is referred to as AHL synthase refers to a protein that has an activity that can include any one, and preferably more than one, of the following characteristics: (a) interacts with (e.g., by binding to) a substrate of a naturally occurring AHL synthase or close variant thereof (e.g., SAM, acyl-ACP, acylated coenzymeA, or acylated phosphopantetheine, or other substrate or fragment thereof); (b) enzymatic activity, such as catalyzing the synthesis of acylhomoserine lactones (AHLs); (c) contributes to quorum sensing signal generation in a population of microorganisms expressing the AHL synthase; or (d) changes production of gene products dependent on the transcription factors that bind the AHL, which result in phenotypes such as biofilm formation, virulence factor production, antibiotic production, lipopolysaccharide production, mating or conjugation factor production, or other characterized downstream effects. The biological activity of (c) or (d) associated with the synthesis of AHLs and the signal generation associated with this synthesis can be referred to as downstream biological activities, since they occur downstream of the actual enzymatic activity of the AHL synthase.

An isolated protein (e.g., an isolated AHL synthase), according to the present invention, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein, and particularly, an isolated AHL synthase (including fragments and homologues thereof), is produced recombinantly. The terms “fragment”, “segment” and “portion” can be used interchangeably herein with regard to referencing a part of a protein. It will be appreciated that, as a result of the determination of the teritiary structure of two AHL synthases herein, various portions of an AHL synthase will now be appreciated as being particularly valuable for mutational analyses and various biological assays outside of the computer-assisted drug design methods disclosed herein. Such portions of AHL synthases and methods of using such portions are explicitly contemplated to be part of the present invention.

Reference to a protein from a specific organism, such as a “Pseudomonas AHL synthase”, by way of example, refers to an AHL synthase (including a homologue of a naturally occurring AHL synthase) from a Pseudomonas microbe or to an AHL synthase that has been otherwise produced from the knowledge of the primary structure (e.g., sequence) and/or the tertiary structure of a naturally occurring AHL synthase from Pseudomonas. In other words, a Pseudomonas AHL synthase includes any AHL synthase that has the structure and function of a naturally occurring AHL synthase from Pseudomonas or that has a structure and function that is sufficiently similar to a Pseudomonas AHL synthase such that the AHL synthase is a biologically active (i.e., has biological activity) homologue of a naturally occurring AHL synthase from Pseudomonas. As such, a Pseudomonas AHL synthase, by way of example, can include purified, partially purified, recombinant, mutated/modified and synthetic proteins.

Proteins of the present invention are preferably retrieved, obtained, and/or used in “substantially pure” form. As used herein, “substantially pure” refers to a purity that allows for the effective use of the protein in vitro, ex vivo or in vivo according to the present invention. For a protein to be useful in an in vitro, ex vivo or in vivo method according to the present invention, it is substantially free of contaminants, other proteins and/or chemicals that might interfere or that would interfere with its use in a method disclosed by the present invention, or that at least would be undesirable for inclusion with the protein when it is used in a method disclosed by the present invention. For example, for an AHL synthase, such methods include crystallization of the protein, use of all or a portion of the protein for mutational analysis, for antibody production, for agonist/antagonist identification assays, and all other methods disclosed herein. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

As used herein, a “structure” of a protein refers to the components and the manner of arrangement of the components to constitute the protein. The “three dimensional structure” or “tertiary structure” of the protein refers to the arrangement of the components of the protein in three dimensions. Such term is well known to those of skill in the art. It is also to be noted that the terms “tertiary” and “three dimensional” can be used interchangeably.

The present invention provides the atomic coordinates that define the three dimensional structure of an AHL synthase. First, the present inventors have determined the atomic coordinates that define the three dimensional structure of a crystalline EsaI AHL synthase from Pantoea stewartii, including the structure of the native EsaI, an EsaI-rhenate complex, and an EsaI-phospho pantetheine (see Example 1 for details). Second, the present inventors have determined the atomic coordinates that define the three dimensional structure of a crystalline LasI mutant (active enzyme) as described in Example 2. Using the guidance provided herein, one of skill in the art will be able to reproduce any of such structures and define atomic coordinates of such a structure.

Example 1 describes the production of an AHL synthase, EsaI, arranged in a crystalline manner in a space group p43 so as to form a unit cell of dimensions a=b=66.40 Å, c=47.33 Å. The atomic coordinates determined from this crystal structure and defining the three dimensional structure of the acyl-homoserinelactone synthase EsaI-rhenate complex are provided as Table 2. The atomic coordinates for the EsaI-rhenate complex in Table 2 were deposited with the Protein Data Bank (PDB), operated by the Research Collaboratory for Structural Bioinformatics (RCSB) (H. M. Berman, J. Westbrook, Z. Feng, G. Gilliland, T. N. Bhat, H. Weissig, I. N. Shindyalov, P. E. Boume, The Protein Data Bank; Nucleic Acids Research, 28:235-242 (2000)), under PDB Deposit No. 1k4j on Oct. 8, 2001, and such coordinates are incorporated herein by reference. The native EsaI crystal is arranged in a space group p4₃ so as to form a unit cell of dimensions a=b=66.99 Å, c=47.01 Å (see Example 1). The atomic coordinates for the EsaI native structure have also been determined and are provided as Table 3. The atomic coordinates for native EsaI were deposited with the Protein Data Bank (PDB) under PDB Deposit No. 1kzf on Feb. 6, 2002, and such coordinates are incorporated herein by reference. The EsaI-phosphopantetheine structure was modeled and is discussed in Example 1 and the atomic coordinates representing this structure are provided as Table 4.

Example 2 describes the production of a LasI mutant (SEQ ID NO:82) arranged in a crystalline manner in a space group F23, so as to form a unit cell of dimensions a=b=c=154.90 Å. The atomic coordinates defining this crystal structure are provided as Table 5.

One embodiment of the present invention includes an AHL synthase in crystalline form. The present invention specifically exemplifies crystalline EsaI and crystalline LasI, both AHL synthases. As used herein, the terms “crystalline AHL synthase” and “AHL synthase crystal” both refer to crystallized AHL synthase and are intended to be used interchangeably. Preferably, a crystalline AHL synthase is produced using the crystal formation method described herein, in particular according to the method disclosed in Example 1 or Example 2. An AHL synthase crystal of the present invention can comprise any crystal structure that comes from crystals formed in any of the allowable spacegroups for proteins (61 of them) and preferably crystallizes as an orthorhombic crystal lattice. In one aspect, a crystalline EsaI of the present invention includes EsaI molecules arranged in a crystalline manner in a space group p4₃ of the tetragonal crystal lattice so as to form a unit cell having approximate dimensions of a=b=66.40 Å, c=47.33 Å, or in a space group p4₃ so as to form a unit cell having approximate dimensions of a=b=66.99 A, c=47.01 Å. In one aspect, a crystalline LasI of the present invention includes LasI molecules arranged in a crystalline manner in a space group F23 of the cubic crystalline lattice, so as to form a unit cell having approximate dimensions of a=b=c=154.90 Å. According to the present invention, a unit cell having “approximate dimensions of” a given set of dimensions refers to a unit cell that has dimensions that are within plus (+) or minus (−) 2.0% of the specified unit cell dimensions. Such a small variation is within the scope of the invention since one of skill in the art could obtain such variance by performing X-ray crystallography at different times on the same crystal. In one embodiment, a crystalline AHL synthase of the present invention has the specified unit cell dimensions set forth above. A preferred crystal of the present invention provides X-ray diffraction data for determination of atomic coordinates of the AHL synthase to a resolution of about 4.0 Å, and preferably to about 3.2 Å, and preferably to about 3.0 Å, and more preferably to about 2.3 Å, and more preferably to about 2.0 Å, and even more preferably to about 1.8 Å.

One embodiment of the present invention includes a method for producing crystals of an AHL synthase, including EsaI and LasI, comprising combining the AHL synthase with a mother liquor and inducing crystal formation to produce the AHL synthase crystals. Although the production of crystals of two AHL synthases are specifically described herein, it is to be understood that such processes as are described herein can be adapted by those of skill in the art to produce crystals of other AHL synthases, such as those listed in Table 1.

By way of example, crystals of EsaI can be formed using a solution containing about 6 mg/ml of EsaI in a mother liquor. A suitable mother liquor of the present invention comprises A suitable mother liquor of the present invention comprises the solution used for crystallization as described in Examples 1 or 2 that causes the protein to crystallize. It could be anything, but for EsaI it was as described in the method. There is some tolerance in the mother liquor conditions so that changes of up to 30% in buffer concentrations, PEG concentrations, isopropanol concentrations 0.5 pH units, and temperatures of between 10° C. and 28° C. can still yield crystals. Supersaturated solutions comprising an AHL synthase can be induced to crystallize by several methods including, but not limited to, vapor diffusion, liquid diffusion, batch crystallization, constant temperature and temperature induction or a combination thereof. Preferably, supersaturated solutions of AHL synthase are induced to crystallize by hanging drop vapor diffusion. In a vapor diffusion method, an AHL synthase molecule is combined with a mother liquor as described above that will cause the protein solution to become supersaturated and form crystals at a constant temperature. Vapor diffusion is preferably performed under a controlled temperature and, by way of example, can be performed at 18° C.

The crystalline AHL synthases of the present invention are analyzed by X-ray diffraction and, based on data collected from this procedure, models are constructed which represent the tertiary structure of the AHL synthase. Therefore, one embodiment of the present invention includes a representation, or model, of the three dimensional structure of an AHL synthase, such as a computer model. A computer model of the present invention can be produced using any suitable software modeling program, including, but not limited to, the graphical display program O (Jones et. al., Acta Crystallography, vol. A47, p. 110, 1991), the graphical display program GRASP, MOLSCRIPT 2.0 (Avatar Software AB, Heleneborgsgatan 21C, SE-11731 Stockholm, Sweden), the program CONTACTS from the CCP4 suite of programs (Bailey, 1994, Acta Cryst. D50:760-763), or the graphical display program INSIGHT. Suitable computer hardware useful for producing an image of the present invention are known to those of skill in the art (e.g., a Silicon Graphics Workstation).

A representation, or model, of the three dimensional structure of the AHL synthase for which a crystal has been produced can also be determined using techniques which include molecular replacement or SIR/MIR (single/multiple isomorphous replacement), or MAD (multiple wavelength anomalous diffraction) methods (Hendrickson et al., 1997, Methods Enzymol., 276:494-522). Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; and Bentley, Meth. Enzym., vol. 276, pp. 611-619, 1997, each of which are incorporated by this reference herein in their entirety) and are performed in a software program including, for example, AmoRe (CCP4, Acta Cryst. D50, 760-763 (1994), SOLVE (Terwilliger et al., 1999, Acta Crystallogr., D55:849-861), RESOLVE (Terwilliger, 2000, Acta Crystallogr., D56:965-972) or XPLOR. Briefly, X-ray diffraction data is collected from the crystal of a crystallized target structure. The X-ray diffraction data is transformed to calculate a Patterson function. The Patterson function of the crystallized target structure is compared with a Patterson function calculated from a known structure (referred to herein as a search structure). The Patterson function of the crystallized target structure is rotated on the search structure Patterson function to determine the correct orientation of the crystallized target structure in the crystal. The translation function is then calculated to determine the location of the target structure with respect to the crystal axes. Once the crystallized target structure has been correctly positioned in the unit cell, initial phases for the experimental data can be calculated. These phases are necessary for calculation of an electron density map from which structural differences can be observed and for refinement of the structure. Preferably, the structural features (e.g., amino acid sequence, conserved di-sulphide bonds, and β-strands or β-sheets) of the search molecule are related to the crystallized target structure.

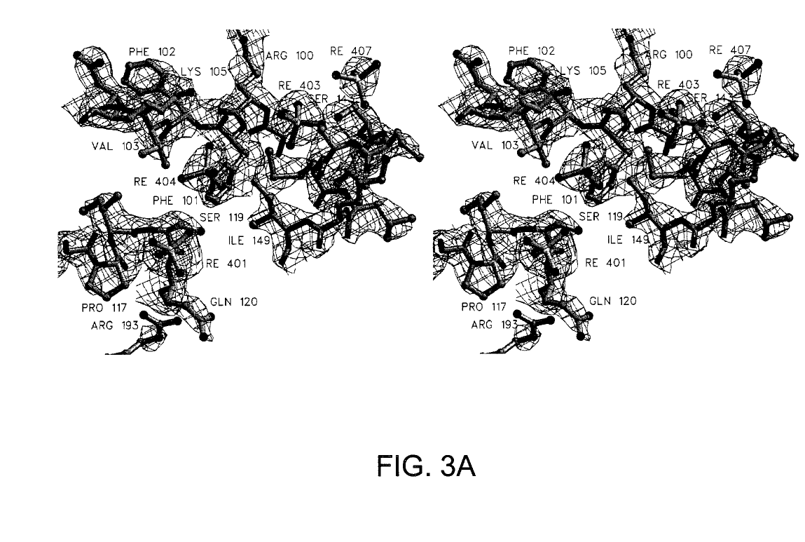

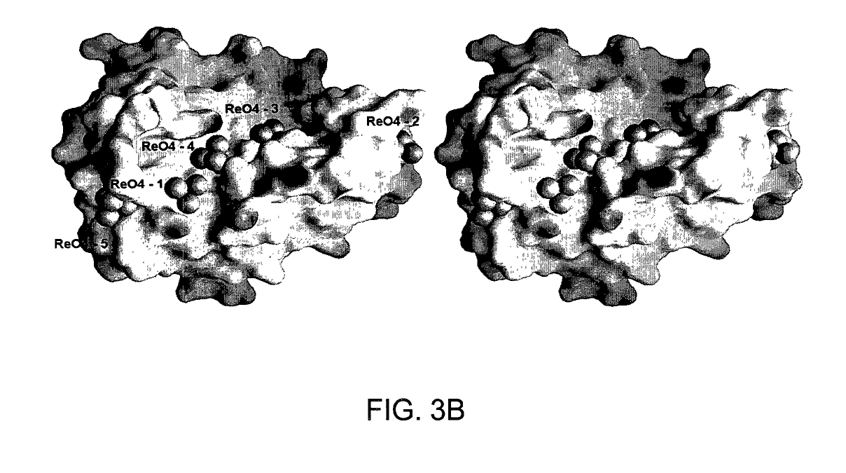

As used herein, the term “model” refers to a representation in a tangible medium of the three dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, and Biosym Technologies, San Diego, Calif. The phrase “providing a picture of the model” refers to the ability to generate a “hard copy” of the model. Hard copies include both motion and still pictures. Computer screen images and pictures of the model can be visualized in a number of formats including space-filling representations, a carbon traces, ribbon diagrams and electron density maps. A variety of such representations of the AHL synthase structural model are shown, for example, in FIGS. 3-5.

Preferably, a three dimensional structure of an AHL synthase provided by the present invention includes:

(a) a structure defined by atomic coordinates of a three dimensional structure of a crystalline AHL synthase (e.g., crystalline EsaI or crystalline LasI);

(b) a structure defined by atomic coordinates selected from:

(i) atomic coordinates represented in any one of Tables 2-5;

(ii) atomic coordinates that define a three dimensional structure having an average root-mean-square deviation (RMSD) of equal to or less than about 1.7 Å over the backbone atoms in secondary structure elements of at least 50% of the residues in a three dimensional structure represented by the atomic coordinates of (1);

wherein the structure has an amino acid sequence comprising at least three of eight conserved amino acid residues corresponding to the following residues in SEQ ID NO:1: Arg²⁴, Phe²⁸, Trp³⁴, Asp⁴⁵, Asp⁴⁸, Ag⁶⁸, GlU⁹⁷, or Arg¹⁰⁰ or to the following residues in SEQ ID NO:2: Arg²³, Phe²⁷, Trp³³, Asp⁴⁴, Asp47, Arg⁷⁰, Glu¹⁰¹ or Arg¹⁰⁴; and

wherein the structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; or

(iii) atomic coordinates in any one of Tables 2-5 defining a portion of the AHL synthase, wherein the portion of the AHL synthase comprises sufficient structural information to perform step (b);

(c) a structure defined by atomic coordinates defining the three dimensional structure of EsaI molecules arranged in a crystalline manner in a space group p4₃ so as to form a unit cell having approximate dimensions of a=b=66.40, c=47.33;

(d) a structure defined by atomic coordinates defining the three dimensional structure of EsaI molecules arranged in a crystalline manner in a space group p4₃ so as to form a unit cell having approximate dimensions of a=b=66.99, c=47.01; or

(e) atomic coordinates defining the three dimensional structure of LasI molecules arranged in a crystalline manner in a space group F23, so as to form a unit cell having approximate dimensions of a=b=c=154.90 Å.

The crystalline AHL synthases, including crystalline EsaI or crystalline LasI, have been described in detail above, as well as methods to produce, analyze and model the structure of such crystals (see also Examples 1 and 2). In addition, the atomic coordinates of Tables 2-5, which define the tertiary structures of several AHL synthases and AHL synthase complexes have also been discussed above (see also Examples 1 and 2).

In one aspect as described above, a three dimensional structure of an AHL synthase provided by the present invention includes a structure wherein the structure has an average root-mean-square deviation (RMSD) of equal to or less than about 1.7 Å over the backbone atoms in secondary structure elements of at least 50% of the residues in a three dimensional structure represented by the atomic coordinates of any one of Tables 2-5. Such a structure can be referred to as a structural homologue of the AHL synthase structures defined by one of Tables 2-5. Preferably, the structure has an average root-mean-square deviation (RMSD) of equal to or less than about 1.6 Å over the backbone atoms in secondary structure elements of at least 50% of the residues in a three dimensional structure represented by the atomic coordinates of any one of Tables 2-5, or equal to or less than about 1.5 Å, or equal to or less than about 1.4 Å, or equal to or less than about 1.3 Å, or equal to or less than about 1.2 Å, or equal to or less than about 1.1 Å, or equal to or less than about 1.0 Å, or equal to or less than about 0.9 Å, or equal to or less than about 0.8 Å, or equal to or less than about 0.7 Å, or equal to or less than about 0.6 Å, or equal to or less than about 0.5 Å, or equal to or less than about 0.4 Å, or equal to or less than about 0.3 Å, or equal to or less than about 0.2 Å, over the backbone atoms in secondary structure elements of at least 50% of the residues in a three dimensional structure represented by the atomic coordinates of any one of Tables 2-5. In another aspect, a three dimensional structure of an AHL synthase provided by the present invention includes a structure wherein the structure has the recited RMSD over the backbone atoms in secondary structure elements of at least 75% of the residues in a three dimensional structure represented by the atomic coordinates of any one of Tables 2-5, and more preferably at least about 80%, and more preferably at least about 85%, and more preferably at least about 90%, and more preferably at least about 95%, and most preferably, about 100% of the residues in a three dimensional structure represented by the atomic coordinates of any one of Tables 2-5.

In one embodiment, the RMSD of a structural homologue of an AHL synthase can be extended to include atoms of amino acid side chains. As used herein, the phrase “common amino acid side chains” refers to amino acid side chains that are common to both the structural homologue and to the structure that is actually represented by such atomic coordinates (e.g., a structure represented by one of Tables 2-5). Preferably, at least 50% of the structure has an average root-mean-square deviation (RMSD) from common amino acid side chains in a three dimensional structure represented by the atomic coordinates of one of Tables 2-5 of equal to or less than about 1.7 Å, or equal to or less than about 1.6 Å, equal to or less than about 1.5 Å, or equal to or less than about 1.4 Å, or equal to or less than about 1.3 Å, or equal to or less than about 1.2 Å, or equal to or less than about 1.1 Å, or equal to or less than about 1.0 Å, or equal to or less than about 0.9 Å, or equal to or less than about 0.8 Å, or equal to or less than about 0.7 Å, or equal to or less than about 0.6 Å, or equal to or less than about 0.5 Å, or equal to or less than about 0.4 Å, or equal to or less than about 0.3 Å, or equal to or less than about 0.2 Å. In another embodiment, a three dimensional structure of an AHL synthase provided by the present invention includes a structure wherein at least about 75% of such structure has the recited average root-mean-square deviation (RMSD) value, and more preferably, at least about 85% of such structure has the recited average root-mean-square deviation (RMSD) value, and most preferably, about 95% of such structure has the recited average root-mean-square deviation (RMSD) value.

In addition to having the recited RMSD values, a structural homologue of an AHL synthase should additionally meet the following criteria for amino acid sequence homology, both of which have been discussed in detail previously herein. First, the structure should represent a protein having an amino acid sequence comprising at least three of the eight absolutely conserved amino acid residues of a LuxI type AHL synthase. In EsaI, these correspond to the following residues in SEQ ID NO: 1: Arg²⁴, Phe²⁸, Trp³⁴, Asp⁴⁵, ASp⁴⁸, Arg⁶⁸, Glu⁹⁷, or Arg¹⁰⁰. In LasI, these correspond to the following residues in SEQ ID NO:2: Arg²³, Phe²⁷, Trp³³, Asp⁴⁴, Asp⁴⁷, Arg⁷⁰, Glu¹⁰¹ or Arg¹⁰⁴. In addition, the structure should represent a protein having an amino acid sequence that has at least three regions having detectable sequence homology with the first three regions (blocks) of the four conserved regions or blocks of sequence homology that have been identified for LuxI type AHL synthases (described above). For EsaI, the first three blocks of conserved sequence homology are found, with respect to SEQ ID NO: 1, at positions: amino acid residues 19 through 56, amino acid residues 63-83, and amino acid residues 90-101. For LasI, the first three regions of conserved sequence homology are found, with respect to SEQ ID NO:2, at positions: amino acid residues 18-55, amino acid residues 65-85 and amino acid residues 95-105. For a given amino acid sequence or amino acid residue to correspond to an amino acid region or amino acid position in another sequence, the position of the sequence or residue in the query sequence should align to the position of the region or residue in the compared sequence using a standard alignment program in the art, but particularly, using the programs BLOCKS (GIBBS) and/or MAST (Henikoff et al., 1995, Gene, 163, 17-26; Henikoff et al., 1994, Genomics, 19, 97-107), using standard manufacturer defaults.

Another structure that is useful in the methods of the present invention is a structure that is defined by the atomic coordinates in any one of Tables 2-5 defining a portion of the AHL synthase, wherein the portion of the AHL synthase comprises sufficient structural information to perform structure based drug design (described below). Suitable portions of an AHL synthase that could be-modeled and used in structure based drug design will be apparent to those of skill in the art. The present inventors have provided at least one example in the coordinates of Table 4, which define the EsaI-phosphopantetheine structure. The present inventors have also identified multiple sites of interest based on the structure of EsaI and LasI (described in detail below). Structures comprising these portions (e.g., the phosphopantetheine core fold of the protein) would be encompassed by the present invention.

Accordingly, one embodiment of the present invention relates to a method of structure-based identification of compounds that regulate the activity of an AHL synthase. Such compounds can regulate the ability of the AHL synthase to bind to a substrate and/or the biological activity of the AHL synthase, such as the enzymatic activity. The method is typically a computer-assisted method of structure based drug design, and includes the steps of: (a) obtaining atomic coordinates that define the three dimensional structure of an AHL synthase, including any of the AHL synthase three dimensional structures or atomic coordinates described herein; and (b) selecting candidate compounds for binding to said AHL synthase by performing structure based drug design with said structure of (a), wherein said step of selecting is performed in conjunction with computer modeling. In one embodiment, step (b) of the method is a step of selecting candidate compounds that inhibit the biological activity of an AHL synthase.

The structures and atomic coordinates used to perform the above-described method have been described in detail above and in the Examples section, and include any structural homologues of AHL synthases described herein. According to the present invention, the phrase “obtaining atomic coordinates that define the three dimensional structure of an AHL synthase” is defined as any means of obtaining providing, supplying, accessing, displaying, retrieving, or otherwise making available the atomic coordinates defining any three dimensional structure of the AHL synthase as described herein. For example, the step of obtaining can include, but is not limited to, accessing the atomic coordinates for the structure from a database or other source; importing the atomic coordinates for the structure into a computer or other database; displaying the atomic coordinates and/or a model of the structure in any manner, such as on a computer, on paper, etc.; and determining the three dimensional structure of an AHL synthase described by the present invention de novo using the guidance provided herein.

The second step of the method of structure based identification of compounds of the present invention includes selecting a candidate compound for binding to and/or inhibiting the biological activity of the AHL synthase represented by the structure model by performing structure based drug design with the model of the structure. According to the present invention, the step of “selecting” can refer to any screening process, modeling process, design process, or other process by which a compound can be selected as useful for binding or inhibiting the activity of an AHL synthase according to the present invention. Methods of structure based identification of compounds are described in detail below. As discussed above, AHL synthases catalyze the synthesis of molecules that are pivotal for quorum sensing signal generation, and therefore, the selection of compounds that compete with, disrupt or otherwise inhibit the biological activity of AHL synthases are highly desirable. Such compounds can be designed using structure based drug design using models of the structures disclosed herein. Until the discovery of the three dimensional structure of the present invention, the only information available for the development of therapeutic compounds based on the AHL synthases was based on the primary sequence of the AHL synthase and mutagenesis studies directed to the isolated protein.

Structure based identification of compounds (e.g., structure based drug design, structure based compound screening, or structure based structure modeling) refers to the prediction or design of a conformation of a peptide, polypeptide, protein (e.g., an AHL synthase), or to the prediction or design of a conformational interaction between such protein, peptide or polypeptide, and a candidate compound, by using the three dimensional structure of the peptide, polypeptide or protein. Typically, structure based identification of compounds is performed with a computer (e.g., computer-assisted drug design, screening or modeling). For example, generally, for a protein to effectively interact with (e.g., bind to) a compound, it is necessary that the three dimensional structure of the compound assume a compatible conformation that allows the compound to bind to the protein in such a manner that a desired result is obtained upon binding. Knowledge of the three dimensional structure of the AHL synthase enables a skilled artisan to design a compound having such compatible conformation, or to select such a compound from available libraries of compounds and/or structures thereof. For example, knowledge of the three dimensional structure of the ACP binding site of AHL synthase enables one of skill in the art to design or select a compound structure that is predicted to bind to the AHL synthase at that site and result in, for example, inhibition of the binding of ACP to a synthase and thereby inhibit a biological response such as AHL production catalyzed by the synthase. In addition, for example, knowledge of the three dimensional structure of an AHL synthase enables a skilled artisan to design an analog of AHL synthase or an analog of an AHL synthase substrate.

Suitable structures and models useful for structure based drug design are disclosed herein. Preferred target structures to use in a method of structure based drug design include any representations of structures produced by any modeling method disclosed herein, including molecular replacement and fold recognition related methods.

According to the present invention, the step of selecting or designing a compound for testing in a method of structure based identification of the present invention can include creating a new chemical compound structure or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of an AHL synthase by computer (e.g. as described by Humblet and Dunbar, Animal Reports in Medicinal Chemistry, vol. 28, pp. 275-283, 1993, M Venuti, ed., Academic Press). The compound itself, if identified as a suitable candidate by the method of the invention, can be synthesized and tested directly with the AHL synthase protein in a biological assay. Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory) and are discussed herein.

Various other methods of structure-based drug design are disclosed in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In the present method of structure based identification of compounds, it is not necessary to align the structure of a candidate chemical compound (i.e., a chemical compound being analyzed in, for example, a computational screening method of the present invention) to each residue in a target site (target sites will be discussed in detail below). Suitable candidate chemical compounds can align to a subset of residues described for a target site. Preferably, a candidate chemical compound comprises a conformation that promotes the formation of covalent or noncovalent crosslinking between the target site and the candidate chemical compound. In one aspect, a candidate chemical compound binds to a surface adjacent to a target site to provide an additional site of interaction in a complex. When designing an antagonist (i.e., a chemical compound that inhibits the biological activity of an AHL synthase), for example, the antagonist should bind with sufficient affinity to the target binding site or substantially prohibit a ligand (e.g., a molecule that specifically binds to the target site) from binding to a target site. It will be appreciated by one of skill in the art that it is not necessary that the complementarity between a candidate chemical compound and a target site extend over all residues specified here in order to inhibit or promote binding of a ligand.

In general, the design of a chemical compound possessing stereochemical complementarity can be accomplished by techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

One embodiment of the present invention for structure based drug design comprises identifying a chemical compound that complements the shape of an AHL synthase, including a portion of AHL synthase. Such method is referred to herein as a “geometric approach”. In a geometric approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule, such as a ligand).

The geometric approach is described by Kuntz et al., J. Mol. Biol., vol. 161, p. 269,1982, which is incorporated by this reference herein in its entirety. The algorithm for chemical compound design can be implemented using the software program DOCK Package, Version 1.0 (available from the Regents of the University of California). Pursuant to the Kuntz algorithm, the shape of the cavity or groove on the surface of a structure (e.g., AHL synthase) at a binding site or interface is defined as a series of overlapping spheres of different radii. One or more extant databases of crystallographic data (e.g., the Cambridge Structural Database System maintained by University Chemical Laboratory, Cambridge University, Lensfield Road, Cambridge CB21EW, U.K.) or the Protein Data Bank maintained by Brookhaven National Laboratory, is then searched for chemical compounds that approximate the shape thus defined.

Chemical compounds identified by the geometric approach can be modified to satisfy criteria associated with chemical complementarity, such as hydrogen bonding, ionic interactions or Van der Waals interactions.

Another embodiment of the present invention for structure based identification of compounds comprises determining the interaction of chemical groups (“probes”) with an active site at sample positions within and around a binding site or interface, resulting in an array of energy values from which three dimensional contour surfaces at selected energy levels can be generated. This method is referred to herein as a “chemical-probe approach.” The chemical-probe approach to the design of a chemical compound of the present invention is described by, for example, Goodford, J. Med. Chem., vol. 28, p. 849, 1985, which is incorporated by this reference herein in its entirety, and is implemented using an appropriate software package, including for example, GRID (available from Molecular Discovery Ltd., Oxford OX2 9LL, U.K.). The chemical prerequisites for a site-complementing molecule can be identified at the outset, by probing the active site of an AHL synthase, for example, (e.g., as represented by the atomic coordinates shown in one of Tables 2-5) with different chemical probes, e.g., water, a methyl group, an amine nitrogen, a carboxyl oxygen and/or a hydroxyl. Preferred sites for interaction between an active site and a probe are determined. Putative complementary chemical compounds can be generated using the resulting three dimensional pattern of such sites.

According to the present invention, suitable candidate compounds to test using the method of the present invention include proteins, peptides or other organic molecules, and inorganic molecules. Suitable organic molecules include small organic molecules. Peptides refer to small molecular weight compounds yielding two or more amino acids upon hydrolysis. A polypeptide is comprised of two or more peptides. As used herein, a protein is comprised of one or more polypeptides. Preferred therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Preferably, a compound that is identified by the method of the present invention originates from a compound having chemical and/or stereochemical complementarity with a site on an AHL synthase. Such complementarity is characteristic of a compound that matches the surface of the enzyme either in shape or in distribution of chemical groups and binds to AHL synthase to inhibit binding of a substrate to the AHL synthase, for example, or to otherwise inhibit the biological activity of the synthase and/or inhibit quorum sensing signal generation in a cell expressing the AHL synthase upon the contact of the compound with the AHL synthase. More preferably, a compound that binds to a ligand binding site on an AHL synthase associates with an affinity of at least about 10⁻⁶ M, and more preferably with an affinity of at least about 10⁻⁷M, and more preferably with an affinity of at least about 10⁻⁸ M.

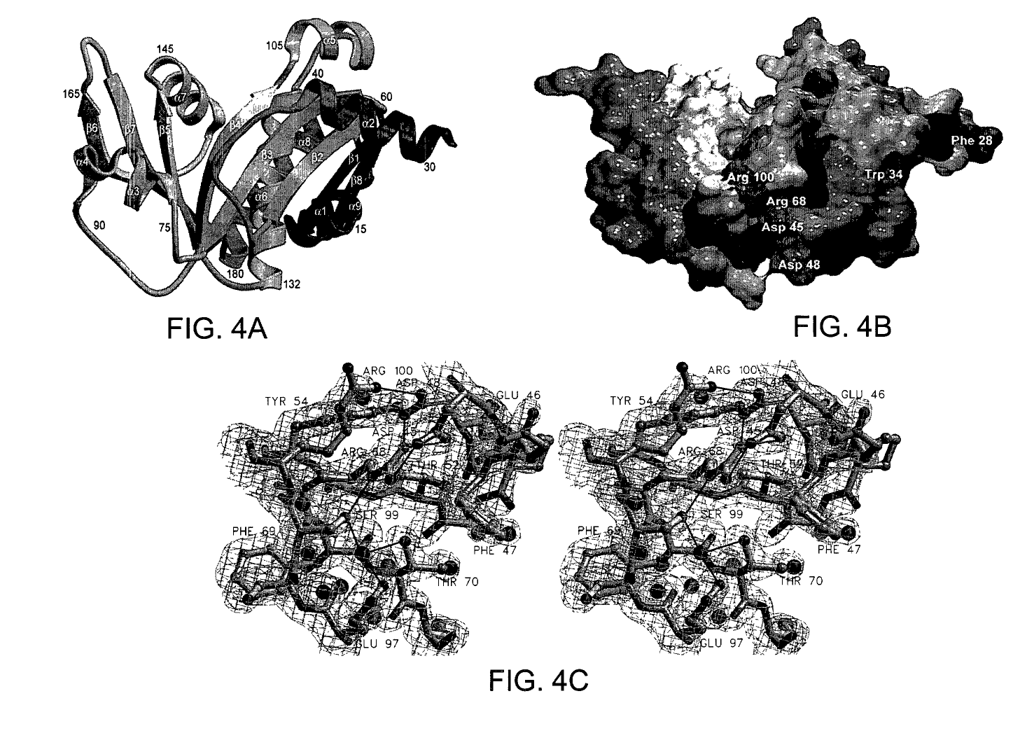

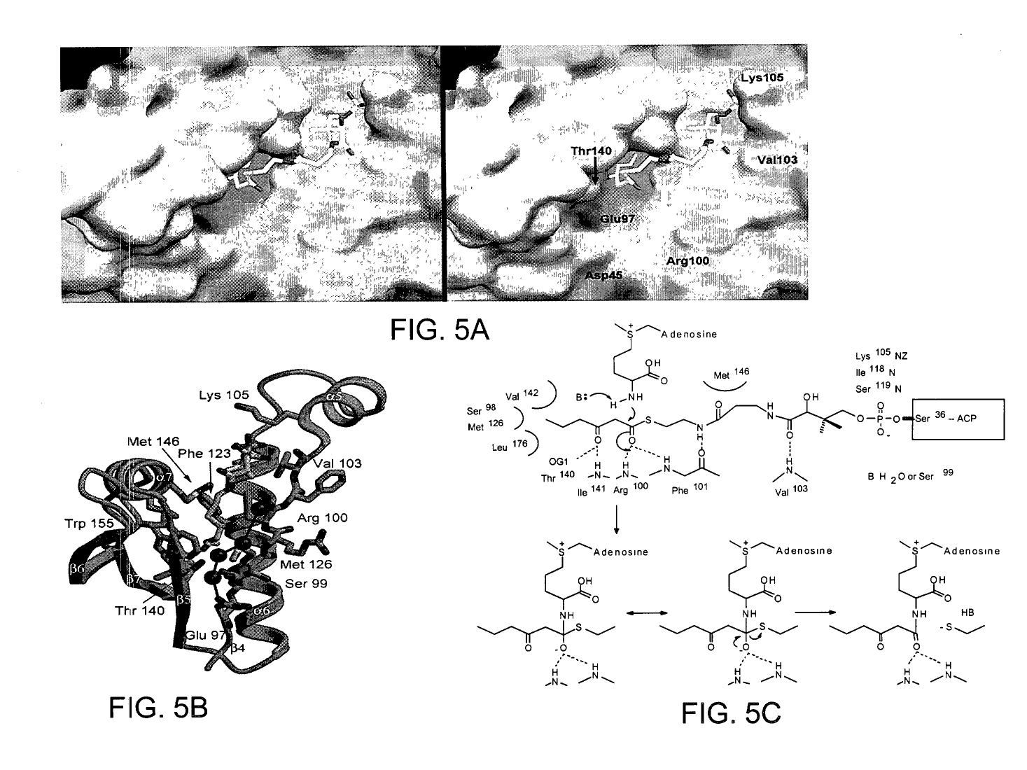

Preferably, the following general sites of an AHL synthase are targets for structure based drug design or identification of candidate compounds and lead compounds (i.e., target sites), although other sites may become apparent to those of skill in the art. The preferred sites include: (1) the phosphopantetheine core fold of the AHL protein (Table 4) (e.g., for EsaI, the core fold is defined as the residues that superimpose to within 2.0 A, and has an RMSD of 0.9 Å over the Cα positions of 71 residues when superimoposed on the GCN5 protein 1); (2) the phosphopantetheine core binding fold of the AHL synthase, which are defined herein as the secondary structure elements in common between EsaI and LasI from the structural alignment (e.g., see FIG. 2); (3) the acyl chain binding region of the AHL synthase; (4) the acyl-ACP binding site of the AHL synthase; (5) the SAM binding site of the AHL synthase; and/or (6) the electrostatic cluster of the AHL synthase. Combinations of any of these general sites are also suitable target sites. These sites are generally referenced with regard to the tertiary structure of the sites. Even if some of such sites were generally known or hypothesized to be important sites prior to the present invention based on the linear sequence and mutational analysis or binding studies of AHL synthases, the present invention actually defines the sites in three dimensions and confirms or newly identifies residues that are important targets that could not be confirmed or identified prior to the present invention. The use of any of these target sites as a three dimensional structure is novel and encompassed by the present invention. Many of these target sites for EsaI are further described and illustrated in the Figures and Examples of the invention. FIG. 4C shows the electrostatic cluster of conserved residues. FIG. 5A is a stereodiagram of acyl-phosphopantetheine modeled into the EsaI active-site cavity (the electrostatic surface is shaded, indicating various charged regions of the surface). FIG. 5B shows the EsaI structure, where the acylation cleft of EsaI and relevant residues and the modeled phosphopantheteine are shown, and where the well-ordered water molecules observed in the native structure that lie along β4 are shown as spheres.

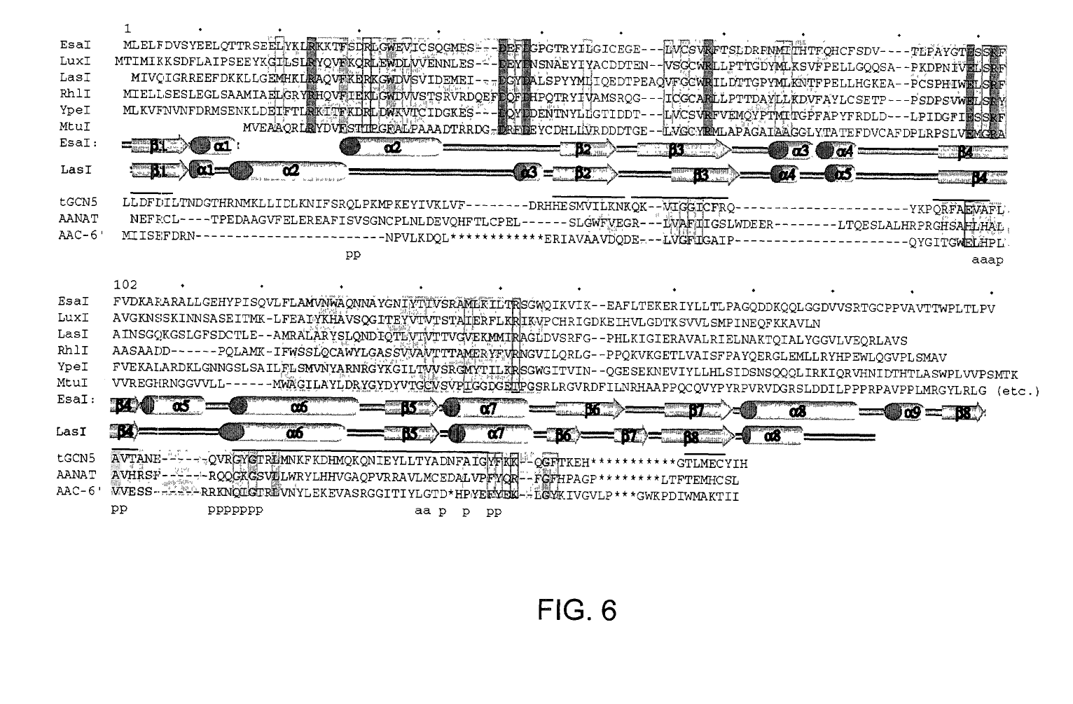

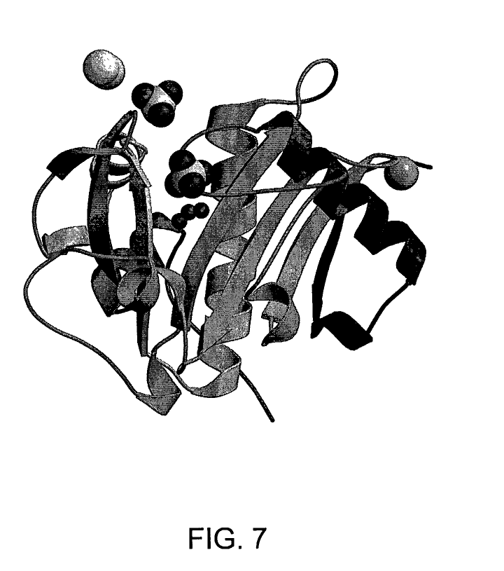

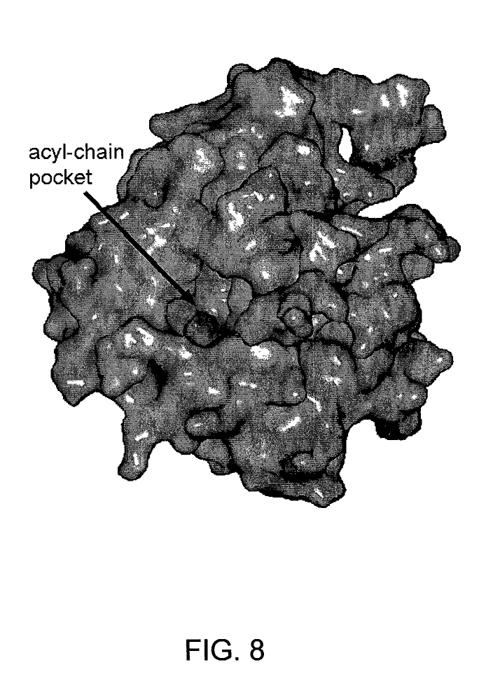

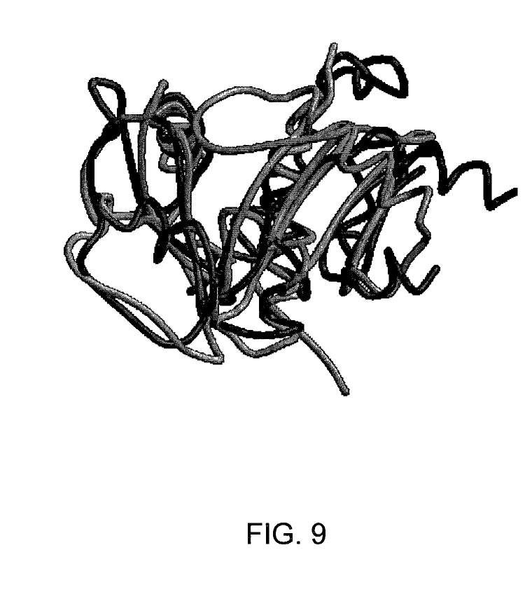

The Examples section and the following discussion provides specific detail regarding the structure of AHL synthases and target sites of AHL synthases based on the three-dimensional structures described for EsaI and the enzymatically active LasI mutant, including the identification of important residues in the structures. It is to be understood, however, that one of skill in the art, using the description of these specific AHL synthase structures provided herein, will be able to identify compounds that are potential candidates for modulating the biological activity of these and other AHL synthases. FIGS. 2 and 6 illustrate how one of skill in the art can now create an alignment of AHL synthases based on both sequence and structural characteristics learned from the present invention and thereby reveal structurally and/or functionally important amino acid residues and target regions on AHL synthases other than EsaI and LasI. All such embodiments are encompassed by the present invention.

Particularly preferred Esa I residues that could be targeted for inhibitor design include, but are not limited to (with respect to SEQ ID NO: 1): (1) residues in the acyl chain binding region, including, but not limited to amino acid positions 98, 99, 119, 123, 138, 140, 142, 146, 149, 150, 153, 155, 176; (2) residues in the acyl-ACP site, including, but not limited to, amino acid positions 148, 151, 152, 180, 181; (3) residues in the SAM site, including, but not limited to 27, 28, 31, 34, 67, 101, 103, 105, 116, 141-143; (4) residues in the electrostatic cluster, including, but not limited to 24, 31, 45, 48, 68, 97, 100. Particularly preferred residues to target in the EsaI structure include, but are not limited to: residues 97-105, 126, 138-157, and/or 174-176, or surface accessible residues likely to be good targets of drug binding, including but not limited to amino acid residues 3, 5, 6, 8-32, 34-36, 38, 39, 53, 58, 77, 77-84, 99-102, 104-111, 119, 131, 132, 136, 137, 143-149, 151, 152, 158-162, 168-171, 175, 177, 179-181, 183-185, 188, 189, 191-193, 197-200, 205, 207, 209, 210.

Particularly preferred residues of LasI that could be targeted for inhibitor design include, but are not limited to: (1) residues in the acyl chain binding region, including 185, 154, 152, 149, 118, 122, 175, 137, 148, 181, 184, 145, 99, 100, 139, 141; (2) residues in the acyl-ACP site, including 180, 151, 147, 150; (3) residues in the SAM site, including 33, 30, 114, 26, 27, 142, 145, 141, 140, 104, 106, 102, 66; (4) residues in the electrostatic cluster, including 20, 8, 42, 23, 47, 49, 67, 53, 101, 100 (all positions given relative to SEQ ID NO:82). In one aspect of the invention, preferred residues to target in the LasI structure include, but are not limited to surface accessible residues likely to be good targets of drug binding, including amino acid residues 1-10, 13-15, 17, 18, 21, 24, 25, 27-41, 43, 45, 47, 49, 57, 70, 78, 82, 83, 96, 105, 119, 120, 123, 124, 127, 128, 130, 135, 136, 143, 144, 147, 148, 150-153, 155, 157, 158, 162-165, 168, 169, 174, 176, 178-180, 182-184.

A candidate compound for binding to or otherwise modulating the activity of an AHL synthase, including to one of the preferred target sites described above, is identified by one or more of the methods of structure-based identification discussed above. As used herein, a “candidate compound” refers to a compound that is selected by a method of structure-based identification described herein as having a potential for binding to an AHL synthase on the basis of a predicted conformational interaction between the candidate compound and the target site of the AHL synthase. The ability of the candidate compound to actually bind to an AHL synthase can be determined using techniques known in the art, as discussed in some detail below. A “putative compound” is a compound with an unknown regulatory activity, at least with respect to the ability of such a compound to bind to and/or regulate an AHL synthase as described herein. Therefore, a library of putative compounds can be screened using structure based identification methods as discussed herein, and from the putative compounds, one or more candidate compounds for binding to or mimicking the target AHL synthase (see embodiments regarding identification of AHL synthase homologues described below) can be identified. Alternatively, a candidate compound for binding to or mimicking an AHL synthase can be designed de novo using structure based drug design, also as discussed above.

Accordingly, in one aspect of the present invention, the method of structure-based identification of compounds that potentially bind to or modulate (regulate) the activity of an AHL synthase further includes steps which confirm whether or not a candidate compound has the predicted properties with respect to its effect on the actual AHL synthase. In one embodiment, the candidate compound is predicted to be an inhibitor of the binding of an AHL synthase to at least one of its substrates, and the method further includes producing or otherwise obtaining a candidate compound selected in the structure based method and determining whether the compound actually has the predicted effect on the AHL synthase or its biological activity. For example, one can additionally contact the candidate compound selected in the structure based identification method with the AHL synthase or a fragment thereof under conditions in which the AHL synthase binds to its substrate in the absence of the candidate compound; and measuring the binding affinity of the AHL synthase or fragment thereof for its substrate or a fragment thereof. In this example (binding), a candidate inhibitor compound is selected as a compound that inhibits the binding of AHL synthase to its substrate when there is a decrease in the binding affinity of the AHL synthase or fragment thereof for the substrate or fragment thereof, as compared to in the absence of the candidate inhibitor compound.

In another embodiment, the candidate compound is predicted to inhibit the biological activity of an AHL synthase, and the method further comprises contacting the actual candidate compound selected by the structure-based identification method with AHL synthase or a targeted fragment thereof, under conditions wherein in the absence of the compound, AHL synthase is biologically active and measuring the ability of the candidate compound to inhibit the activity of the AHL synthase.

In another embodiment, the candidate compound, or modeled AHL synthase structure in some embodiments (described below), is predicted to be a mimic or homologue of a natural AHL synthase and is predicted to have modified biological activity as compared to the natural AHL synthase. For example, one can model and then produce and test an AHL synthase homologue that has different substrate specificity as compared to the natural AHL synthase, or a homologue that increased or decreased biological activity as compared to the natural AHL synthase. Such homologues can be useful in various biological assays, as competitive inhibitors, or in the production of genetically engineered organisms, such as plants and microbes. For example, in one embodiment, plant-produced natural AHLs (i.e., as a result of transgenic expression of an AHL synthase or AHL synthase homologue according to the present invention) may modulate the behavior of the bacterial pathogen and cause it to express quorum sensing regulated genes prematurely.

In one embodiment, the conditions under which an AHL synthase according to the present invention is contacted with a candidate compound, such as by mixing, are conditions in which the enzyme is not stimulated (activated) or bound to a natural ligand (substrate) if essentially no candidate compound is present. In one aspect, a natural stimulant or substrate can be added after contact with the candidate compound to determine the effect of the compound on the biological activity of the AHL synthase. Alternatively, this aspect can be designed simply to determine whether the candidate compound binds to the AHL synthase (i.e., in the absence of any additional testing, such as by addition of substrates). For example, such conditions include normal culture conditions in the absence of a stimulatory compound or substrate.

In another embodiment, the conditions under which an AHL synthase according to the present invention is contacted with a candidate compound, such as by mixing, are conditions in which the enzyme is normally bound by a substrate or activated if essentially no candidate compound is present. Such conditions can include, for example, contact of the AHL synthase with the appropriate substrates or other stimulatory molecule. In this embodiment, the candidate compound can be contacted with the AHL synthase prior to the contact of the AHL synthase with the substrates (e.g., to determine whether the candidate compound blocks or otherwise inhibits the binding of the AHL synthase to the substrates or the biological activity of the AHL synthase), or after contact of the AHL synthase with the substrates (e.g., to determine whether the candidate compound downregulates, or reduces the biological activity of the AHL synthase after the initial contact with the substrates).

The present methods involve contacting the AHL synthase with the candidate compound being tested for a sufficient time to allow for binding to, activation or inhibition of the enzyme by the candidate compound. The period of contact with the candidate compound being tested can be varied depending on the result being measured, and can be determined by one of skill in the art. For example, for binding assays, a shorter time of contact with the candidate compound being tested is typically suitable, than when activation is assessed. As used herein, the term “contact period” refers to the time period during which the AHL synthase is in contact with the compound being tested. The term “incubation period” refers to the entire time during which cells expressing the AHL synthase, for example, are allowed to grow or incubate prior to evaluation, and can be inclusive of the contact period. Thus, the incubation period includes all of the contact period and may include a further time period during which the compound being tested is not present but during which growth or cellular events are continuing (in the case of a cell based assay) prior to scoring. It will be recognized that shorter incubation times are preferable because compounds can be more rapidly screened.

In accordance with the present invention, a cell-based assay is conducted under conditions that are effective to screen candidate compounds selected in the structure-based identification method to confirm whether such compounds are useful as predicted. Effective conditions include, but are not limited to, appropriate media, temperature, pH and oxygen conditions that permit the growth of the cell that expresses the AHL synthase. An appropriate, or effective, medium refers to any medium in which a cell that naturally or recombinantly expresses an AHL synthase, when cultured, is capable of cell growth and expression of the AHL synthase. Such a medium is typically a solid or liquid medium comprising growth factors and assimilable carbon, nitrogen, sulfur and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

Cells that are useful in the cell-based assays of the present invention include any cell that expresses the AHL synthase of interest and particularly, other components of a quorum sensing system. Such cells include bacteria and mycobacteria and particularly, gram negative bacteria and more particularly, bacteria or mycobacteria that are or can be pathogenic.

The assay of the present invention can also be a non-cell based assay. In this embodiment, the candidate compound can be directly contacted with an isolated AHL synthase, or a portion thereof (e.g., a portion comprising an acyl chain binding region or a portion comprising a SAM binding region), and the ability of the candidate compound to bind to the enzyme or portion thereof can be evaluated, such as by an immunoassay or other binding assay. The assay can, if desired, additionally include the step of further analyzing whether candidate compounds which bind to the AHL synthase are capable of increasing or decreasing the activity of the AHL synthase. Such further steps can be performed by cell-based assay, as described above, or by a non-cell-based assay that measures enzymatic activity. For example, the AHL synthase can be immobilized on a solid support and evaluated for binding to a candidate compound and additionally, enzyme activity can be measured if the appropriate conditions and substrates are provided. Enzymes can be immobilized on a substrate such as: artificial membranes, organic supports, biopolymer supports and inorganic supports. The protein can be immobilized on the solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports can be in any suitable form, including in a bead form, plate form, or well form.

In one embodiment, a BIAcore machine can be used to determine the binding constant of a complex between an AHL synthase and a candidate compound or between AHL synthase and a substrate, for example, in the presence and absence of the candidate compound. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the AHL synthase and monitoring the response function (e.g., the change in the refractive index with respect to time) allows the complex dissociation constant to be determined in the presence of the candidate compound.

Other suitable assays for measuring the binding of a candidate compound to an AHL synthase, and/or for measuring the ability of such compound to affect the binding of an AHL synthase to a substrate include, for example, immunoassays such as enzyme linked immunoabsorbent assays (ELISA) and radioimmunoassays (RIA), or determination of binding by monitoring the change in the spectroscopic or optical properties of the AHL synthase or any substrate, through fluorescence, UV absorption, circular dichrosim, or nuclear magnetic resonance (NMR).

Candidate compounds identified by the present invention can include agonists of AHL synthase activity and antagonists of AHL synthase activity, with the identification of antagonists or inhibitors being preferred. As used herein, the phrase “agonist” refers to any compound that interacts with an AHL synthase and elicits an observable response. More particularly, an AHL synthase agonist can include, but is not limited to, a protein (including an antibody), a peptide, a nucleic acid or any suitable product of drug design (e.g., a mimetic) which is characterized by its ability to agonize (e.g., stimulate, induce, increase, enhance) the biological activity of a naturally occurring AHL synthase in a manner similar to a natural agonist (e.g., a natural substrate for the enzyme). An “antagonist” refers to any compound which inhibits the biological activity of AHL synthase and particularly, which inhibits the effect of the interaction of AHL synthase with its natural substrates. More particularly, an AHL synthase antagonist (e.g., an inhibitor) is capable of associating with an AHL synthase such that the biological activity of the enzyme is decreased (e.g., reduced, inhibited, blocked, reversed, altered) in a manner that is antagonistic (e.g., against, a reversal of, contrary to) to the natural activity of the enzyme (e.g., the activity induced under normal conditions in the presence of natural substrates). It is noted that the three dimensional structures disclosed herein can be used to design or identify candidate compounds that agonize or antagonize the biological activity of the AHL synthase. However, it is desirable to inhibit the activity of the AHL synthase in order to decrease the pathogenicity of a microorganism; therefore, the identification or design of antagonists/inhibitors is preferred.

Suitable antagonist (i.e., inhibitory) compounds to identify using the present method are compounds that interact directly with the AHL synthase, thereby inhibiting the binding of a substrate to the AHL synthase, by either blocking the substrate binding site of AHL synthase (referred to herein as substrate analogs) or by modifying other regions of the AHL synthase such that the natural substrate cannot bind to the AHL synthase (e.g., by allosteric interaction) or so that AHL synthase enzymatic activity is inhibited.

An inhibitory compound of the present invention can also include a compound that essentially mimics at least a portion of the AHL synthase, such as the portion that binds to a natural substrate (referred to herein as a peptidomimetic compound). Accordingly, another embodiment of the present invention relates to a method to produce an AHL synthase homologue that catalyzes the synthesis of AHL compounds having antibacterial biological activity. This method includes the steps of: (a) obtaining atomic coordinates that define the three dimensional structure of an AHL synthase, including any of the AHL synthase three dimensional structures or atomic coordinates described herein; (b) performing computer modeling with the atomic coordinates of (a) to identify at least one site in the AHL synthase structure that is predicted to modify the biological activity of the AHL synthase; (c) producing a candidate AHL synthase homologue that is modified in the at least one site identified in (b); and (d) determining whether the candidate AHL synthase homologue of (c) catalyzes the synthesis ofAHL compounds having antibacterial biological activity. In one embodiment, the method includes the step of determining whether a compound has affinity (of a threshold amount stronger than a Kd of 1×10⁻⁶ M) or specificity for the AHL-synthase (e.g., binds to the AHL synthase with greater affinity than to any other protein tested by a factor of greater than 10-fold).

Yet another embodiment of the present invention relates to a method to produce an AHL synthase homologue with modified biological activity as compared to a natural AHL synthase. This method includes the steps of: (a) obtaining atomic coordinates that define the three dimensional structure of an AHL synthase, including any of the AHL synthase three dimensional structures or atomic coordinates described herein; (b) using computer modeling of the atomic coordinates in (a) to identify at least one site in the AHL synthase structure that is predicted to contribute to the biological activity of the AHL synthase; and (c) modifying the at least one site in an AHL synthase protein to produce an AHL synthase homologue which is predicted to have modified biological activity as compared to a natural AHL synthase. The final step of modifying the site on the AHL synthase can be performed by producing a “virtual AHL synthase homologue” on a computer, such as by generating a computer model of an AHL synthase homologue, or by modifying an AHL synthase protein to produce the homologue, such as by classical mutagenesis or recombinant technology.

The atomic coordinates that define the three dimensional structure of an AHL synthase and the step of obtaining such coordinates have been described in detail previously herein with regard to the method of structure based identification of compounds. Computer modeling methods suitable for modeling the atomic coordinates to identify sites in an AHL synthase structure that are predicted to contribute to the biological activity of an AHL synthase, as well as for modeling homologues of an AHL synthase, have been discussed generally above. A variety of computer software programs for modeling and analyzing three dimensional structures of proteins are publicly available. The Examples section describes in detail the use of a few of such programs to analyze the three dimensional structure of EsaI, for example. Such computer software programs include, but are not limited to, the graphical display program O (Jones et. al., Acta Crystallography, vol. A47, p. 110, 1991), the graphical display program GRASP, MOLSCRIPT 2.0 (Avatar Software AB, Heleneborgsgatan 21 C, SE-11731 Stockholm, Sweden), the program CONTACTS from the CCP4 suite of programs (Bailey, 1994, Acta Cryst. D50:760-763), or the graphical display program INSIGHT.

The present inventors have identified multiple sites on the AHL synthases, EsaI and LasI, which are believed to contribute to the biological activity of the AHL synthase. These sites and amino acid positions have been discussed in detail above and in the Examples. Using similar methods of analysis of the AHL synthase model, one can identify or further analyze sites on the AHL synthase or on other AHL synthase models which are predicted to affect (contribute to) the biological activity of the AHL synthase. Such sites will generally include the phosphopantetheine core fold and substrate binding sites.

Once target sites for modification on an AHL synthase are identified, AHL synthase homologues having modifications at these sites can be produced and evaluated to determine the effect of such modifications on AHL synthase biological activity. In one embodiment, an AHL synthase homologue can be modeled on a computer to produce a computer model of an AHL synthase homologue which predicts the effects of given modifications on the structure of the synthase and its subsequent interaction with other molecules. Such computer modeling techniques are well known in the art. By way of example, the present inventors have exemplified such a technique by modeling the acyl-phosphopantetheine model into the active-site cavity of a rigid model of EsaI using CNS (Brünger et al., 1998, Acta Crystallogr., D54:905-921) (See Example 1).

In another aspect, or subsequent to an initial computer generation and evaluation of an AHL synthase homologue model, an actual AHL synthase homologue can be produced and evaluated by modifying target sites of a natural AHL synthase to produce a modified or mutant AHL synthase. Homologues of the present invention can be produced using techniques known in the art including, but not limited to, direct modifications to the protein or modifications to the gene encoding the protein using, for example, classic or recombinant DNA techniques to effect random or targeted mutagenesis. Examples of several AHL synthase homologues which were produced by the present inventors as a result of the structural analysis of the AHL synthase EsaI are provided in Example 1.

One embodiment of the present invention relates to an isolated AHL synthase homologue (e.g., mutant) which comprises at least one amino acid modification as compared to a naturally occurring AHL synthase, or portion of such a homologue that contains the modification. Such a mutant preferably has modified biological activity, including, but not limited to, modified enzymatic activity, modified substrate binding, modified substrate specificity, and/or modified product synthesis as compared to the wild-type AHL synthase, or equivalent fragment/portion of a wild-type AHL synthase. One aspect of this embodiment relates to an isolated protein comprising a mutant AHL synthase, wherein the protein comprises an amino acid sequence that differs from the amino acid sequence of a naturally occurring AHL synthase by at least one amino acid modification. In a particularly preferred embodiment, the modification results in a mutant AHL synthase that catalyzes the production of a different AHL product as compared to the naturally occurring AHL synthase. The present inventors have demonstrated such a mutant AHL synthase in Example 1.

The modifications to the amino acid sequence of the mutant AHL synthase can include any of the modifications to any amino acid position corresponding to any of the target residues identified above for EsaI and Las I. In one embodiment of the invention, a mutant (homologue) AHL synthase is disclosed that has an amino acid sequence comprising at least one modification as compared to a naturally occurring AHL synthase, wherein the modification is in a region selected from: (1) the phosphopantetheine core binding fold of the AHL synthase; (2) the acyl chain binding region of the AHL synthase; (3) the acyl-ACP binding site of the AHL synthase; (4) the SAM binding site of the AHL synthase; and/or (5) the electrostatic cluster of the AHL synthase in the acyl chain binding region of the AHL synthase. In another aspect, the mutant AHL synthase has an amino acid sequence comprising at least one modification, as compared to a naturally occurring AHL synthase, in the acyl chain binding region of the AHL synthase. In yet another aspect, the mutant AHL synthase has an amino acid sequence comprising at least one modification, as compared to a naturally occurring AHL synthase, in an amino acid position corresponding to an amino acid position of SEQ ID NO: 1 selected from: (1) residues in the acyl chain binding region, including, but not limited to amino acid positions 98, 99, 119, 123, 138, 140, 142, 146, 149, 150, 153, 155, 176; (2) residues in the acyl-ACP site, including, but not limited to, amino acid positions 148, 151, 152, 180, 181; (3) residues in the SAM site, including, but not limited to 27, 28, 31, 34, 67, 101, 103, 105, 116, 141-143; (4) residues in the electrostatic cluster, including, but not limited to 24, 31, 45, 48, 68, 97, 100; or (5) any of residues 97-105, 126, 138-157, and/or 174-176, or (6) surface accessible residues likely to be good targets of drug binding, including but not limited to amino acid residues 3, 5, 6, 8-32, 34-36, 38, 39, 53, 58, 77, 77-84, 99-102, 104-111, 119, 131, 132, 136, 137, 143-149, 151, 152, 158-162, 168-171, 175, 177, 179-181, 183-185, 188, 189, 191-193, 197-200, 205, 207, 209, 210. In another aspect, the mutant AHL synthase has an amino acid sequence comprising at least one modification, as compared to a naturally occurring AHL synthase, in an amino acid position corresponding to an amino acid position of SEQ ID NO:82 selected from: (1) residues in the acyl chain binding region, including 185, 154, 152, 149, 118, 122, 175, 137, 148, 181, 184, 145, 99, 100, 139, 141; (2) residues in the acyl-ACP site, including 180, 151, 147, 150; (3) residues in the SAM site, including 33, 30, 114, 26, 27, 142, 145, 141, 140, 104, 106, 102, 66; (4) residues in the electrostatic cluster, including 20, 8, 42, 23, 47, 49, 67, 53, 101, 100; and (5) surface accessible residues likely to be good targets of drug binding, including amino acid residues 1-10, 13-15, 17, 18, 21, 24, 25, 27-41, 43, 45, 47, 49, 57, 70, 78, 82, 83, 96, 105, 119, 120, 123, 124, 127, 128, 130, 135, 136, 143, 144, 147, 148, 150-153, 155, 157, 158, 162-165, 168, 169, 174, 176, 178-180, 182-184. In one aspect, the mutant AHL synthase comprises a mutation in an amino acid residue corresponding to Thr¹⁴⁰ in SEQ ID NO:1. In yet another aspect, the mutant AHL synthase comprises a mutation in an amino acid residue corresponding to Ser⁹⁹ of SEQ ID NO: 1.

One aspect of the invention relates to a mutant EsaI protein, wherein the protein comprises an amino acid sequence that differs from SEQ ID NO: 1 (wild-type EsaI sequence) by an amino acid deletion, substitution, insertion or derivatization that results in a modified or mutant AHL synthase protein. For example, mutant AHL synthases encompassed by the present invention include AHL synthase homologues having an amino acid sequence that differs from the wild-type sequence (SEQ ID NO: 1) by a substitution selected from: a non-arginine amino acid residue at position 24, a non-phenyalanine amino acid residue at position 28, a non-tryptophan amino acid residue at position 34, a non-aspartate amino acid residue at position 45, a non-aspartate amino acid residue at position 48, a non-arginine amino acid residue at position 68, a non-glutamate amino acid residue at position 97, a non-serine amino acid residue at position 99, a non-arginine amino acid residue at position 100; and a non-threonine amino acid residue at position 140. Preferably, the mutant EsaI protein has modified biological activity as compared to a wild-type EsaI protein. Particularly preferred EsaI mutants according to the present invention have an amino acid sequence that differs from the wild-type sequence (SEQ ID NO:1) by a substitution selected from: (1) D⁴⁵N (wherein the D residue is the wild type residue, the number indicates the amino acid position relative to SEQ ID NO: 1, and the N is the substituted residue); (2) E⁹⁷Q; (3) S⁹⁹A; (4) T¹⁴⁰V; and (5) T¹⁴⁰A. These mutants are merely exemplary of the types of homologues that can be produced using the knowledge gained from the structure analysis of an AHL synthase; other modifications will be apparent to those of skill in the art and such homologues are intended to be encompassed by the present invention.

One embodiment of the invention relates to a transgenic microorganism or plant (or part of a plant) comprising one or more cells that recombinantly express a nucleic acid sequence encoding any of the mutant AHL synthases as described herein.

Now that the present inventors have determined the three dimensional structure for two AHL synthases, one of skill in the art can make predictions regarding the structures of related AHL synthases (e.g., see the list of synthases in Table 1) and/or identify other putative proteins that appear to belong to the same structural class of AHL synthases. The present inventors have identified a putative protein of unknown function from Mycobacterium tuberculosis that is believed by the present inventors to be an AHL synthase of the same structural type as the AHL synthases (e.g., EsaI and LasI) described in the present invention. This protein was disclosed as a hypothetical protein among several open reading frames in a Sep. 7, 2001 database submission of genome sequence for Mycobacterium tuberculosis (Accession No. NC_{—}000962.1). The open reading frame that encodes what the present inventors believe is a novel AHL synthase from M tuberculosis, is designated in the database submission as a region encoding a hypothetical protein of unknown function. The amino acid sequence for the hypothetical protein is provided in Accession No. NP_{—}217543. The present inventors have designated this Mycobacterial tuberculosis protein, represented herein by SEQ ID NO:67, as MtuI. FIG. 6 shows an alignment and topology (based on knowledge gained from the structural characterization of EsaI and LasI) of several known AHL synthases and MtuI, the putative AHL synthase from Mycobacterium tuberculosis. As shown in FIG. 6, MtuI shares conserved residues and regions of significant homology with the known AHL synthases which the inventors believe have the structure signature represented by EsaI and LasI. To the present inventors' knowledge, the MtuI protein has never been isolated, expressed or identified by function prior to this invention. Mycobacterial proteins having significant homology to MtuI have now also been identified by the present inventors in M. bovis, M. leprae, and M. avium. Other open reading frames that show homology to the M. tuberculosis MtuI protein of the present invention, particularly with regard to AHL synthase signature regions (e.g., conserved regions discussed in detail above), and which may represent additional AHL synthases from other bacteria, are listed in Table 1 B and include:

TABLE 1B

| | |
|---|---|
| gi\|13882902\|gb\|AE007130.1\|AE007130 Mycobacterium tuberculosis | (SEQ ID NO:68; also in TABLE 1A) |
| gi\|20520937\|emb\|AL133469.2\|SCM10 Streptomyces coelicolor | (SEQ ID NO:83) |
| gi\|17546257\|ref\|NP_519659.1\|Ralstonia solanacearum | (SEQ ID NO:84) |
| gi\|17978632\|gb\|AAL47567.1\|Burkholderia thailandensis | (SEQ ID NO:85) |
| gi\|15599546\|ref\|NP_253040.1\|Pseudomonas aeruginosa | (SEQ ID NO:86) |
| gi\|17988083\|ref\|NP_540717.1\|Brucella melitensis | (SEQ ID NO:87) |
| gi\|17934260\|ref\|NP_531050.1\|Agrobacterium tumefaciens str. C58 | (SEQ ID NO:88) |
| gi\|17227724\|ref\|NP_484272.1\|Nostoc sp. PCC 7120 | (SEQ ID NO:89) |
| M. avium 432 Frame 4:gnl\|TIGR\|M.avium_432 Mycobacterium avium | (SEQ ID NO:90) |
| M. bovis contig 636 Frame 1 | (SEQ ID NO:91) |
| gi\|14023393\|dbj\|AP003001.2\|AP003001 Mesorhizobium loti | (SEQ ID NO:92) |
| gnl\|CBCUMN_1770\|mycpara_Contig1332 Mycobacterium avium | (SEQ ID NO:93) |
| gnl\|TIGR_1773_2\|mtub210_69 Mycobacterium tuberculosis | (SEQ ID NO:94) |
| gnl\|Sanger_1765\|mbovis_Contig281 Mycobacterium bovis | (SEQ ID NO:95) |
| gnl\|TIGR_1772\|msmeg_3272 Mycobacterium smegmatis | (SEQ ID NO:96) |
| gnl\|TIGR_1772\|msmeg_3267 Mycobacterium smegmatis | (SEQ ID NO:97) |
| gnl\|Sanger_518\|bbronchi_Contig162 Bordetella bronchiseptica | (SEQ ID NO:98) |
| gnl\|Sanger_519\|bparaper_Contig76 Bordetella parapertussis | (SEQ ID NO:99) |
| EsaI Erwinia stewartii | (SEQ ID NO:100) |

Therefore, another embodiment of the present invention relates to an isolated AHL synthase comprising an amino acid sequence selected from the group consisting of: (a) an amino acid sequence that is at least about 40% identical to an amino acid sequence chosen from any of SEQ ID NO:67 or SEQ ID NO:83-100, wherein the amino acid sequence has AHL synthase activity; and (b) a fragment of an amino acid sequence of (a), wherein the fragment has AHL synthase activity. Preferably, the amino acid sequence is 40% identical to amino acid sequence (e.g., SEQ ID NO:67) over the full length of the amino acid sequence, wherein the protein has AHL synthase biological activity. In another aspect, an isolated AHL synthase of the present invention has an amino acid sequence that is at least about 45% identical, and even more preferably at least about 50% identical, and even more preferably at least about 55% identical, and even more preferably at least about 60% identical, and even more preferably at least about 65% identical, and even more preferably at least about 70% identical, and even more preferably at least about 75% identical, and even more preferably at least about 80% identical, and even more preferably at least about 85% identical, and even more preferably at least about 90% identical and even more preferably at least about 95% identical, and even more preferably at least about 96% identical, and even more preferably at least about 97% identical, and even more preferably at least about 98% identical, and even more preferably at least about 99% identical to an amino acid sequence chosen from: any of SEQ ID NO:67 or SEQ ID NO:83-100, over the full length of the amino acid sequence, wherein the protein has AHL synthase biological activity.

In one embodiment, an isolated AHL synthase of the present invention, in addition to having the above-identified identity to the amino acid sequence chosen from: any of SEQ ID NO:67 or SEQ ID NO:83-100, has at least a detectable homology with an amino acid sequence that corresponds to at least one, and preferably two, and more preferably three, and even more preferably four, of the conserved blocks of sequences known for LuxI type AHL synthases (described above and illustrated for several synthases in FIG. 2). In one embodiment, an AHL synthase homologue has an amino acid sequence that is at least about 20% identical to an amino acid sequence that corresponds to at least one, and preferably two, and more preferably three, and even more preferably four, of these conserved blocks of sequences. More preferably, an AHL synthase homologue has an amino acid sequence that is at least about 25% identical, and more preferably at least about 30% identical, and more preferably at least about 35% identical, and more preferably at least about 40% identical, and more preferably at least about 45% identical, and more preferably at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical, and more preferably at least about 90% identical, and more preferably at least about 95% identical, to an amino acid sequence that corresponds to at least one, and preferably two, and more preferably three, and even more preferably four, of these conserved blocks of sequences.

In another embodiment, an isolated AHL synthase of the present invention, in addition to having the above-identified identity to an amino acid sequence chosen from: any of SEQ ID NO:67 or SEQ ID NO:83-100, has an amino acid sequence comprising at least three and more preferably four, and more preferably five, and more preferably six, and more preferably seven, and even more preferably eight, out of eight absolutely conserved amino acid residues in LuxI type AHL synthases (described in detail above and specifically shown for several AHL synthases—see FIG. 2).

In another embodiment, an isolated AHL synthase of the present invention has an amino acid sequence that is at least about 70% identical to an amino acid sequence chosen from: any of SEQ ID NO:67 or SEQ ID NO:83-100, over at least 50 amino acids of the amino acid sequence chosen from: any of SEQ ID NO:67 or SEQ ID NO:83-100. More preferably, an isolated AHL synthase of the present invention has an amino acid sequence that is at least about 75% identical, and more preferably at least about 80% identical, and more preferably at least about 85% identical, and more preferably at least about 90% identical and more preferably at least about 95% identical, and more preferably at least about 96% identical, and more preferably at least about 97% identical, and more preferably at least about 98% identical, and more preferably at least about 99% identical to an amino acid sequence chosen from: any of SEQ ID NO:67 or SEQ ID NO:83-100, over at least 75 amino acids, and more preferably 100 amino acids, and more preferably 125, and more preferably 150, and more preferably 175, and more preferably 200, and more preferably 225 amino acids of the amino acid sequence chosen from: any of SEQ ID NO:67 or SEQ ID NO:83-100. In a most preferred embodiment, such a protein has AHL synthase biological activity.

In one embodiment of the present invention, an AHL synthase according to the present invention has an amino acid sequence that is less than about 100% identical to an amino acid sequence chosen from: any of SEQ ID NO:67 or SEQ ID NO:83-100. In another aspect of the invention, an AHL synthase according to the present invention has an amino acid sequence that is less than about 99% identical to an amino acid sequence chosen from: any of SEQ ID NO:67 or SEQ ID NO:83-100, and in another embodiment, is less than 98% identical to an amino acid sequence chosen from: any of SEQ ID NO:67 or SEQ ID NO:83-100, and in another embodiment, is less than 97% identical to an amino acid sequence chosen from: any of SEQ ID NO:67 or SEQ ID NO:83-100, and in another embodiment, is less than 96% identical to an amino acid sequence chosen from: any of SEQ ID NO:67 or SEQ ID NO:83-100, and in another embodiment, is less than 95% identical to an amino acid sequence chosen from: any of SEQ ID NO:67 or SEQ ID NO:83-100, and in another embodiment, is less than 94% identical to an amino acid sequence chosen from: any of SEQ ID NO:67 or SEQ ID NO:83-100, and in another embodiment, is less than 93% identical to an amino acid sequence chosen from: any of SEQ ID NO:67 or SEQ ID NO:83-100, and in another embodiment, is less than 92% identical to an amino acid sequence chosen from: any of SEQ ID NO:67 or SEQ ID NO:83-100, and in another embodiment, is less than 91% identical to an amino acid sequence chosen from: any of SEQ ID NO:67 or SEQ ID NO:83-100, and in another embodiment, is less than 90% identical to an amino acid sequence chosen from: any of SEQ ID NO:67 or SEQ ID NO:83-100.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Sch\ddot{aa}ffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) PSI-BLAST with the standard default parameters (Position-Specific Iterated BLAST; or (4) any of the software programs/algorithms described in the Examples or elsewhere herein. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows:

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

Other methods for aligning sequences (e.g., BLOCKS and MAST) have been discussed above.

An AHL synthase of the present invention can also include proteins having an amino acid sequence comprising at least 30 contiguous amino acid residues of an amino acid sequence chosen from: any of SEQ ID NO:67 or SEQ ID NO:83-100, (e.g., 30 contiguous amino acid residues having 100% identity with 30 contiguous amino acids of SEQ ID NO:67). In a preferred embodiment, an AHL synthase of the present invention includes proteins having amino acid sequences comprising at least 50, and more preferably at least 75, and more preferably at least 100, and more preferably at least 115, and more preferably at least 130, and more preferably at least 150, and more preferably at least 200 contiguous amino acid residues of an amino acid sequence chosen from: any of SEQ ID NO:67 or SEQ ID NO:83-100. In one embodiment, such a protein has AHL synthase biological activity.

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, an AHL synthase of the present invention, including an AHL synthase homologue, includes a protein having an amino acid sequence that is sufficiently similar to a naturally occurring AHL synthase amino acid sequence that a nucleic acid sequence encoding the homologue is capable of hybridizing under moderate, high, or very high stringency conditions (described below) to (i.e., with) a nucleic acid molecule encoding the naturally occurring AHL synthase (i.e., to the complement of the nucleic acid strand encoding the naturally occurring AHL synthase amino acid sequence). Preferably, a AHL synthase is encoded by a nucleic acid sequence that hybridizes under moderate, high or very high stringency conditions to the complement of a nucleic acid sequence that encodes a protein comprising an amino acid sequence chosen from: any of SEQ ID NO:67 or SEQ ID NO:83-100. Such hybridization conditions are described in detail below. A nucleic acid sequence complement of nucleic acid sequence encoding an AHL synthase of the present invention refers to the nucleic acid sequence of the nucleic acid strand that is complementary to the strand which encodes the AHL synthase. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence of an AHL synthase, and/or with the complement of the nucleic acid sequence that encodes any of such amino acid sequences. Methods to deduce a complementary sequence are known to those skilled in the art. It should be noted that since amino acid sequencing and nucleic acid sequencing technologies are not entirely error-free, the sequences presented herein, at best, represent apparent sequences of AHL synthases of the present invention.

In another embodiment, an AHL synthase can include any AHL synthases that are structural homologues of the EsaI and LasI AHL synthases described above.

A preferred protein of the present invention comprises an isolated AHL synthase from a mycobacterium. Such mycobacteria can include, but are not limited to mycobacteria of the species: M tuberculosis, M. avium, M. bovis, and M. leprae. A particularly preferred protein of the present invention comprises an amino acid sequence chosen from: any of SEQ ID NO:67 or SEQ ID NO:83-100, or a fragment of such sequence that has AHL synthase biological activity.

AHL synthase homologues can, in one embodiment, be the result of natural allelic variation or natural mutation. AHL synthase homologues can also be naturally occurring AHL synthase from different organisms (e.g., other mycobacteria or bacteria) with at least 30% identity to one another at the nucleic acid or amino acid level as described herein. AHL synthase homologues of the present invention can also be produced using techniques known in the art including, but not limited to, direct modifications to the protein or modifications to the gene encoding the protein using, for example, classic or recombinant DNA techniques to effect random or targeted mutagenesis. A naturally occurring allelic variant of a nucleic acid encoding a given AHL synthase is a gene that occurs at essentially the same locus (or loci) in the genome as the gene which encodes the given AHL synthase, but which, due to natural variations caused by, for example, mutation or recombination, has a similar but not identical sequence. Natural allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

AHL synthases of the present invention also include expression products of gene fusions (for example, used to overexpress soluble, active forms of the recombinant protein), of mutagenized genes (such as genes having codon modifications to enhance gene transcription and translation), and of truncated genes (such as genes having membrane binding domains removed to generate soluble forms of a membrane protein, or genes having signal sequences removed which are poorly tolerated in a particular recombinant host).

The minimum size of a protein and/or homologue of the present invention is, in one aspect, a size sufficient to have AHL synthase biological activity. In another embodiment, a protein of the present invention is at least 30 amino acids long, and more preferably, at least about 50, and more preferably at least 75, and more preferably at least 100, and more preferably at least 115, and more preferably at least 130, and more preferably at least 150, and more preferably at least 200 amino acids long. There is no limit, other than a practical limit, on the maximum size of such a protein in that the protein can include a portion of an AHL synthase or a full-length AHL synthase, plus additional sequence (e.g., a fusion protein sequence), if desired.

The present invention also includes a fusion protein that includes an AHL synthase-containing domain (i.e., an amino acid sequence for an AHL synthase according to the present invention) attached to one or more fusion segments. Suitable fusion segments for use with the present invention include, but are not limited to, segments that can: enhance a protein's stability; provide other desirable biological activity; and/or assist with the purification of a AHL synthase (e.g., by affinity chromatography). A suitable fusion segment can be a domain of any size that has the desired function (e.g., imparts increased stability, solubility, biological activity; and/or simplifies purification of a protein). Fusion segments can be joined to amino and/or carboxyl termini of the AHL synthase-containing domain of the protein and can be susceptible to cleavage in order to enable straight-forward recovery of a AHL synthase. Fusion proteins are preferably produced by culturing a recombinant cell transfected with a fusion nucleic acid molecule that encodes a protein including the fusion segment attached to either the carboxyl and/or amino terminal end of an AHL synthase-containing domain.

One embodiment of the present invention relates to an isolated nucleic acid molecule comprising a nucleic acid sequence that encodes an AHL synthase of the present invention including the putative AHL synthase disclosed as MtuI (SEQ ID NO:67), or any of the amino acid sequences represented by SEQ ID NOs:83-100 homologues of such sequence, and nucleic acid sequences fully complementary thereto. A nucleic acid molecule encoding an AHL synthase of the present invention includes a nucleic acid molecule encoding any of the AHL synthases, including homologues, discussed above.

In one embodiment, nucleic acid molecules encoding an AHL synthase of the present invention include isolated nucleic acid molecules that hybridize under moderate stringency conditions, and even more preferably under high stringency conditions, and even more preferably under very high stringency conditions with the complement of a nucleic acid sequence encoding a naturally occurring AHL synthase. Preferably, an isolated nucleic acid molecule encoding an AHL synthase of the present invention comprises a nucleic acid sequence that hybridizes under moderate or high stringency conditions to the complement of a nucleic acid sequence that encodes a protein comprising an amino acid sequence chosen from: any of SEQ ID NO:67 or SEQ ID NO:83-100.

As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tm of a particular hybrid, and wash conditions typically include a combination of salt and temperature conditions that are approximately 12-20° C. below the calculated Tm of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

In one embodiment, a nucleic acid sequence can be used as a probe or primer to identify and/or clone other nucleic acid sequences encoding AHL synthases. Such a nucleic acid sequence can vary in size from about 8 nucleotides up to, including all whole integers in between, 500 nucleotides. In another embodiment, the present invention includes an isolated nucleic acid molecules comprising a nucleic acid sequence encoding a protein having an amino acid sequence comprising at least 30 contiguous amino acid residues of an amino acid sequence chosen from: any of SEQ ID NO:67 or SEQ ID NO:83-100, (i.e., 30 contiguous amino acid residues having 100% identity with 30 contiguous amino acids of any of such amino acid sequences). In a preferred embodiment, an isolated nucleic acid molecule comprises a nucleic acid sequence encoding a protein having an amino acid sequence comprising at least 50, and more preferably at least 75, and more preferably at least 100, and more preferably at least 115, and more preferably at least 130, and more preferably at least 150, and more preferably at least 200, contiguous amino acid residues of an amino acid sequence chosen from: any of SEQ ID NO:67 or SEQ ID NO:83-100. Such a protein preferably has AHL synthase biological activity.

In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene, such as an AHL synthase gene. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid-sequence in nature (i.e., are heterologous sequences). Isolated nucleic acid molecules can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

A nucleic acid molecule homologue can be produced using a number of methods known to those skilled in the art (see, for example, Sambrook et al., ibid.). For example, nucleic acid molecules can be modified using a variety of techniques including, but not limited to, classical mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

Any of the AHL synthases described herein, including homologues, can be produced with from at least one, and up to about 20, additional heterologous amino acids flanking each of the C- and/or N-terminal end of the AHL synthase protein. Such a protein can be referred to as “consisting essentially of” a given AHL synthase amino acid sequence. According to the present invention, the heterologous amino acids are a sequence of amino acids that are not naturally found (i.e., not found in nature, in vivo) flanking the AHL synthase sequence or which would not be encoded by the nucleotides that flank the naturally occurring AHL synthase nucleic acid sequence as it occurs in the gene, if such nucleotides in the naturally occurring sequence were translated using standard codon usage for the organism from which the AHL synthase is derived. Similarly, the phrase “consisting essentially of”, when used with reference to a nucleic acid sequence herein, refers to a nucleic acid sequence encoding a AHL synthase (including fragments/homologues) that can be flanked by from at least one, and up to as many as about 60, additional heterologous nucleotides at each of the 5′ and/or the 3′ end of the nucleic acid sequence encoding the AHL synthase. The nucleotides are not naturally found (i.e., not found in nature, in vivo) flanking the AHL synthase coding sequence as it occurs in the natural gene.

Another embodiment of the present invention includes a recombinant nucleic acid molecule comprising a recombinant vector and a nucleic acid sequence encoding an AHL synthase, or a biologically active subunit or homologue/mutant (including a fragment) thereof, as previously described herein. This embodiment of the present invention also includes AHL synthase regulatory proteins identified by the structure based identification methods provided herein, which can be used as therapeutic compounds in various host cells. The methods described herein are applicable to the recombinant expression of any molecule that forms part of the present invention, including molecules identified using methods of the invention.

Therefore, according to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences including nucleic acid sequences that are not naturally found adjacent to nucleic acid sequence to be delivered, although the vector can also contain regulatory nucleic acid sequences (e.g., promoters, untranslated regions) which are naturally found adjacent to nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of the recombinant host cell. The entire vector can remain in place within a host cell, or under certain conditions, the plasmid DNA can be deleted, leaving behind the nucleic acid molecule encoding an AHL synthase or homologue thereof. The integrated nucleic acid molecule can be under chromosomal promoter control, under native or plasmid promoter control, or under a combination of several promoter controls. Single or multiple copies of the nucleic acid molecule can be integrated into the chromosome.

As used herein, the phrase “recombinant nucleic acid molecule” is used primarily to refer to a recombinant vector into which has been ligated the nucleic acid sequence to be cloned, manipulated, transformed into the host cell (i.e., the insert). “DNA construct” can be used interchangeably with “recombinant nucleic acid molecule” in some embodiments and is further defined herein to be a constructed (non-naturally occurring) DNA molecules useful for introducing DNA into host cells, and the term includes chimeric genes, expression cassettes, and vectors.

In one embodiment, a recombinant vector of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector (e.g., a promoter) which enable the transcription and translation of the nucleic acid sequence within the recombinant host cell.

Typically, a recombinant vector includes at least one nucleic acid molecule of the present invention (e.g., a nucleic acid molecule comprising a nucleic acid sequence encoding an AHL synthase) operatively linked to one or more transcription control sequences to form a recombinant nucleic acid molecule. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence (including the order of the sequences, the orientation of the sequences, and the relative spacing of the various sequences) in a manner such that proteins encoded by the nucleic acid sequence can be expressed when transfected (i.e., transformed, transduced, transfected, conjugated or conduced) into a host cell. Methods of operatively linking expression control sequences to coding sequences are well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989).

Vectors for transferring recombinant sequences into eukaryotic cells are known to the person skilled in the art and include, but are not limited to self-replicating vectors, integrative vectors, artificial chromosomes, Agrobacterium based transformation vectors and viral vector systems such as retroviral vectors, adenoviral vectors or lentiviral vectors.

Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell useful in the present invention.

The transcription control sequences includes a promoter. The promoter may be any DNA sequence which shows transcriptional activity in the chosen host cell or organism. The promoter may be inducible or constitutive. It may be naturally-occurring, may be composed of portions of various naturally-occurring promoters, or may be partially or totally synthetic. The promoter may be a native promoter (i.e., the promoter that naturally occurs within the AHL synthase gene and regulates transcription thereof) or a non-native promoter (i.e., any promoter other than the promoter that naturally occurs within the AHL synthase gene, including other promoters that naturally occur within the chosen host cell). Guidance for the design of promoters is provided by studies of promoter structure, such as that of Harley and Reynolds, Nucleic Acids Res., 15, 2343-61 (1987). Also, the location of the promoter relative to the transcription start may be optimized. See, e.g., Roberts, et al., Proc. Natl Acad. Sci. USA, 76, 760-4 (1979). Many suitable promoters for use in prokaryotes and eukaryotes are well known in the art.

For instance, suitable constitutive promoters for use in plants include, but are not limited to: the promoters from plant viruses, such as the ³⁵S promoter from cauliflower mosaic virus (Odell et al., Nature 313:810-812 (1985), the full length transcript promoter with duplicated enhancer domains from peanut chlorotic streak caulimovirus (Maiti and Shepherd, BBRC 244:440-444 (1998)), promoters of Chlorella virus methyltransferase genes (U.S. Pat. No. 5,563,328), and the full-length transcript promoter from figwort mosaic virus (U.S. Pat. No. 5,378,619); the promoters from such genes as rice actin (McElroy et al., Plant Cell 2:163-171 (1990)), ubiquitin (Christensen et al., Plant Mol. Biol. 12:619-632 (1989) and Christensen et al., Plant Mol. Biol. 18:675-689 (1992)), pEMU (Last et al., Theor. Appl. Genet. 81:581-588 (1991)), MAS (Velten et al., EMBO J. 3:2723-2730 (1984)), maize H3 histone (Lepetit et al., Mol. Gen. Genet. 231:276-285 (1992) and Atanassova et al., Plant Journal 2(3):291-300 (1992)), Brassica napus ALS3 (PCT application WO 97/41228); and promoters of various Agrobacterium genes (see U.S. Pat. Nos. 4,771,002, 5,102,796, 5,182,200, 5,428,147).

Suitable inducible promoters for use in plants include, but are not limited to: the promoter from the ACE1 system which responds to copper (Mett et al. PNAS 90:4567-4571 (1993)); the promoter of the maize In2 gene which responds to benzenesulfonamide herbicide safeners (Hershey et al., Mol. Gen. Genetics 227:229-237 (1991) and Gatz et al., Mol. Gen. Genetics 243:32-38 (1994)), and the promoter of the Tet repressor from Tn10 (Gatz et al., Mol. Gen. Genet. 227:229-237 (1991). A particularly preferred inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone. Schena et al., Proc. Natl. Acad. Sci. USA 88:10421 (1991). Other inducible promoters for use in plants are described in EP 332104, PCT WO 93/21334 and PCT WO 97/06269.

Suitable promoters for use in bacteria include, but are not limited to, the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gene, the Bacillus pumilus xylosidase gene, the phage lambda P_{R} and P_{L} promoters, and the Escherichia coli lac, trp and tac promoters. See PCT WO 96/23898 and PCT WO 97/42320.

Suitable promoters for use in yeast host cells include, but are not limited to, promoters from yeast glycolytic genes, promoters from alcohol dehydrogenase genes, the TP11 promoter, and the ADH2-4c promoter. See, e.g., PCT WO 96/23898.

Finally, promoters composed of portions of other promoters and partially or totally synthetic promoters can be used. See, e.g., Ni et al., Plant J, 7:661-676 (1995) and PCT WO 95/14098 describing such promoters for use in plants.

The promoter may include, or be modified to include, one or more enhancer elements. Preferably, the promoter will include a plurality of enhancer elements. Promoters containing enhancer elements provide for higher levels oftranscription as compared to promoters which do not include them. Suitable enhancer elements for use in plants include the ³⁵S enhancer element from cauliflower mosaic virus (U.S. Pat. Nos. 5,106,739 and 5,164,316) and the enhancer element from figwort mosaic virus (Maiti et al., Transgenic Res., 6, 143-156 (1997)). Other suitable enhancers for use in other cells are known. See PCT WO 96/23898 and Enhancers And Eukaryotic Expression (Cold Spring Harbor Press, Cold Spring Harbor, N.Y., 1983).

Recombinant nucleic acid molecules of the present invention, which can be either DNA or RNA, can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

For efficient expression, the coding sequences are preferably also operatively linked to a 3′ untranslated sequence. The 3′ untranslated sequence contains transcription and/or translation termination sequences. The 3′ untranslated regions can be obtained from the flanking regions of genes from bacterial, plant or other eukaryotic cells. For use in prokaryotes, the 3′ untranslated region will include a transcription termination sequence. For use in plants and other eukaryotes, the 3′ untranslated region will include a transcription termination sequence and a polyadenylation sequence. Suitable 3′ untranslated sequences for use in plants include those of the cauliflower mosaic virus ³⁵S gene, the phaseolin seed storage protein gene, the pea ribulose biphosphate carboxylase small subunit E9 gene, the soybean 7S storage protein genes, the octopine synthase gene, and the nopaline synthase gene.

In plants and other eukaryotes, a 5′ untranslated sequence is typically also employed. The 5′ untranslated sequence is the portion of an mRNA which extends from the 5′CAP site to the translation initiation codon. This region of the mRNA is necessary for translation initiation in eukaryotes and plays a role in the regulation of gene expression. Suitable 5′ untranslated regions for use in plants include those of alfalfa mosaic virus, cucumber mosaic virus coat protein gene, and tobacco mosaic virus.

It will be appreciated by one skilled in the art that use of recombinant DNA technologies can improve control of expression of transformed nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgamo sequences), modification of nucleic acid molecules to correspond to the codon usage of the host cell, and deletion of sequences that destabilize transcripts.

One or more recombinant molecules of the present invention can be used to produce an encoded product (e.g., an AHL synthase or an AHL synthase regulatory protein) of the present invention. In one embodiment, an encoded product is produced by expressing a nucleic acid molecule as described herein under conditions effective to produce the protein. A preferred method to produce an encoded protein is by transfecting (transforming) a host cell with one or more recombinant molecules to form a recombinant host cell. Suitable host cells to transfect include, but are not limited to, any prokaryotic or eukaryotic cell that can be transfected, with bacterial, fungal (e.g., yeast), algal and plant cells being particularly preferred. Host cells can be either untransfected cells or cells that are already transfected with at least one other recombinant nucleic acid molecule.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast, or into plant cells. In microbial systems and plant systems, the term “transformation” is used to describe an inherited change due to the acquisition of exogenous nucleic acids by the microorganism or plant and is essentially synonymous with the term “transfection.” Therefore, transfection techniques include, but are not limited to, transformation, particle bombardment, electroporation, microinjection, chemical treatment of cells, lipofection, adsorption, infection (e.g., Agrobacterium mediated transformation and virus mediated transformation) and protoplast fusion (protoplast transformation). Methods of transforming prokaryotic and eukaryotic host cells are well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); PCT WO 96/23898 and PCT WO 97/42320.

For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10: 1 (1991). Descriptions of Agrobacterium vector systems and methods for Agrobacterium-mediated gene transfer are provided by numerous references, including Gruberetal., supra, Mikietal., supra, Moloneyetal., Plant Cell Reports 8:238 (1989), and U.S. Pat. Nos. 4,940,838 and 5,464,763.

A generally applicable method of plant transformation is microprojectile-mediated transformation wherein DNA is carried on the surface of microprojectiles. The expression vector is introduced into plant tissues with a biolistic device that accelerates the microprojectiles to speeds sufficient to penetrate plant cell walls and membranes. Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

Accordingly, it is the object of the present invention to create genetically modified host cells, and particularly, genetically modified plants or microorganisms, that have introduced modified AHL synthases or AHL synthase regulatory compounds identified by the structure based methods of the present invention. It is one objective of the invention to provide plant produced AHLs, or AHL-like inhibitors, to influence the behavior of plant pathogenic bacteria. In these cases, the presence of AHLs in the plant tissue disrupts the normal disease process. This process may circumvent important steps in the disease developmental process, which seems to parallel biofilm formation Similarly, expression of AHLs in the root system of plants may lead to secretion of the signal into the rhizosphere thus influencing the growth and activity of beneficial bacteria in the rhizosphere. In these cases, an enzyme with increased activity (e.g., an AHL synthase homologue with increased biological activity) is expected to be of great value.

In one embodiment of the invention, AHL synthases, including any of the AHL synthase homologues described herein, are used to produce AHLs for application to combat biofilm formation. For example in case of P. stewartii, Agrobacterium tumefaciens, and Burkholderia cepacia, addition of AHL leads to premature mucoidy, and this in turn prevents bacterial surface attachment. If one could prevent bacterial surface adhesion one would possibly minimize substrate-bound biofilm formation. Therefore, genetically engineered production microorganisms or even cell-free enzyme reaction methods can be used to produce AHLs for use in the prevention of bacterial surface attachment. As one example, such AHL preparations could be used in coatings, such as paints, to protect a surface, such as the surface of a ship or boat, from bacterial biofilms that routinely form on the surface of the ship. Other such applications will be apparent to those of skill in the art.

According to the present invention, a genetically modified microorganism or plant includes a microorganism or plant that has been modified using recombinant technology and/or classical mutagenesis techniques. According to the present invention, genetic modifications that result in an increase in gene expression or function (the preferred embodiment) can be referred to as amplification, overproduction, overexpression, activation, enhancement, addition, or up-regulation of a gene. For example, a genetic modification in a gene encoding AHL synthase which results in an increase in the function of the AHL synthase, can be the result of an increased expression of the AHL synthase, an enhanced activity of the AHL synthase, or an inhibition of a mechanism that normally inhibits the expression or activity of the AHL synthase. Genetic modifications which result in a decrease in gene expression, in the function of the gene, or in the function of the gene product (i.e., the protein encoded by the gene) can be referred to as inactivation (complete or partial), deletion, interruption, blockage, silencing or down-regulation of a gene. For example, a genetic modification in a gene encoding AHL synthase which results in a decrease in the function of the AHL synthase, can be the result of a complete deletion of the gene (i.e., the gene does not exist, and therefore the protein does not exist), a mutation in the gene which results in incomplete or no translation of the protein (e.g., the protein is not expressed), a mutation in the gene or genome which results in silencing of a gene, or a mutation in the gene which decreases or abolishes the natural function of the protein (e.g., a protein is expressed which has decreased or no enzymatic activity).

A recombinant host cell (e.g., a type of genetically modified host cell) is cultured or grown in a suitable medium, under conditions effective to express the recombinant molecule and achieve the desired result. An appropriate, or effective, medium refers to any medium in which a recombinant host cell of the present invention, when cultured, is capable of producing the desired product (e.g., an AHL synthase, a modified AHL synthase, an AHL synthase regulatory compound). Such a medium is typically an aqueous medium comprising assimilable carbon, nitrogen and phosphate sources. Such a medium can also include appropriate salts, minerals, metals and other nutrients. Microorganisms of the present invention can be cultured in conventional fermentation bioreactors. The microorganisms can be cultured by any fermentation process which includes, but is not limited to, batch, fed-batch, cell recycle, and continuous fermentation. Preferred growth conditions for potential host microorganisms according to the present invention are well known in the art. Plants, such as transgenic plants, are cultured in a tissue culture medium or grown in a suitable medium such as soil. An appropriate, or effective, tissue culture medium for recombinant plant cells is known in the art and generally includes similar components as for a suitable medium for the culture of microbial cells (e.g., assimilable carbon, nitrogen and phosphate sources, as well as appropriate salts, minerals, metals and other nutrients). A suitable growth medium for higher plants includes any growth medium for plants, including, but not limited to, soil, sand, any other particulate media that support root growth (e.g. vermiculite, perlite, etc.) or Hydroponic culture, as well as suitable light, water and nutritional supplements which optimize the growth of the higher plant.

Recombinant host cells of the present invention can include any genetically modified microorganisms, host cells of an animal such as a mammal that are treated using gene therapy, and cells of a plant to form a transgenic plant. As described above, the present invention has applications for designing novel AHL synthases to produce altered AHL compounds as antibacterial agents and for commercial production purposes. These novel synthases could be put into transgenic animals, plants or used in gene therapy, for example, to produce altered bacterial behavior. Additionally, the compounds identified using the structure-based approach for identification of modulators of AHL synthases may also be introduced into host cells, transgenic microbes and transgenic plants for therapeutic benefit.

Yet another embodiment of the present invention relates to a method to identify a compound that regulates quorum sensing signal generation using the novel mycobacterial AHL synthase disclosed herein, or homologues thereof, in an assay to detect regulators of this synthase. The method generally includes the steps of: (a) contacting an AHL synthase or biologically active fragment thereof with a putative regulatory compound, wherein the AHL synthase comprises an amino acid sequence that is at least about 70% identical to an amino acid sequence chosen from any of SEQ ID NO:67 or SEQ ID NO:83-100, or a biologically active fragment thereof, wherein the amino acid sequence has AHL synthase activity; and (b) detecting whether the putative regulatory compound increases or decreases a biological activity of the AHL synthase as compared to in the absence of contact with the compound. Compounds that increase or decrease activity of the AHL synthase, as compared to in the absence of the compound, indicates that the putative regulatory compound is a regulator of the AHL synthase. More preferred AHL synthase homologues of an amino acid sequence chosen from any of SEQ ID NO:67 or SEQ ID NO:83-100, have been described above and are also encompassed in this method. Biological activity of an AHL synthase can be evaluated by measuring an activity that includes, but is not limited to, the binding of the AHL synthase to a substrate, AHL enzymatic activity, synthesis of an AHL, quorum sensing signal generation in a population of microorganisms expressing the AHL synthase. Such biological activities and methods of detecting the same have been described above and in the Examples. Other AHL synthases and homologues thereof described herein (including structural homologues) can also be used in such methods.

Methods of identifying candidate compounds and selecting compounds that bind to and activate, inhibit, mimic, modify AHL synthases, including both structural and biological assays, have now been described in detail. Candidate compounds can be synthesized using techniques known in the art, and depending on the type of compound. Synthesis techniques for the production of non-protein compounds, including organic and inorganic compounds are well known in the art.

For smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture.

If larger quantities of a protein are desired, or if the protein is a larger polypeptide, the protein can be produced using recombinant DNA technology. A protein can be produced recombinantly by culturing a cell capable of expressing the protein (i.e., by expressing a recombinant nucleic acid molecule encoding the protein) under conditions effective to produce the protein, and recovering the protein. Effective culture conditions have been described above.

Once a compound has been identified that modulates the biological activity of an AHL synthase according to the present invention, or once a homologue of an AHL synthase with modified biological activity has been identified and produced, such compounds and homologues can be used in any of a variety of therapeutic or beneficial applications. For example, novel AHL synthases can produce altered AHL compounds as antibacterial agents and for commercial production purposes. These novel synthases could be put into transgenic animals, plants or used in gene therapy, for example, to produce altered bacterial behavior. AHL synthase regulatory compounds, and particularly inhibitors, can be used as therapeutic compositions in a variety of organisms, including animals (e.g., mammals) and plants, to inhibit or alter the activity of the AHL synthase, which ideally will have downstream effects of inhibition of the quorum sensing system of bacteria infecting the animals or plants. It has previously been shown that inhibition of components of a quorum sensing system can render microbes having such a system avirulent or attenuated.

Therefore, one embodiment of the present invention relates to a therapeutic composition comprising a compound that inhibits the biological activity of an AHL synthase. The compound is identified either using the structure based method of identification described herein or the biological assays described herein, in the case of inhibitors of the MtuI putative AHL synthase described herein. Further embodiments of the invention relate to methods to treat a disease or condition that can be regulated by modifying the biological activity of an AHL synthase (e.g., a disease or condition caused by a pathogenic microorganism having a quorum sensing system in which an AHL synthase of the present invention is involved). One particular embodiment of the present invention relates to a method to inhibit quorum sensing signal generation in a population of microbial cells, comprising contacting a population of microbial cells that express an AHL synthase with an antagonist of the AHL synthase, wherein the antagonist decreases the biological activity of the AHL synthase, or with an AHL synthase homologue as described herein. The population of microbes can be a population that infects plants or animals. Such methods include genetically modifying microbes, plants or animal cells to contain a therapeutic compound or synthase homologue of the present invention or administering to a microbe, plant or animal cell an AHL regulatory compound. The treatment of plants or animal hosts which may be infected by pathogenic microbes can be performed in conjunction with conventional therapies, such as antibiotic treatment or administration of other antibacterial agents.

A composition, and particularly a therapeutic composition, of the present invention generally includes the therapeutic compound (e.g., the compound identified by the structure based identification method or other method described herein) and a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. Preferred pharmaceutically acceptable carriers are capable of maintaining a compound identified by the present methods in a form that, upon arrival of compound at the cell target in a culture, host cell, plant, or animal, the compound is capable of interacting with its target (e.g., AHL synthase).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

One type ofpharmaceutically acceptable carrier includes a controlled release formulation that is capable of slowly releasing a composition of the present invention into a patient or culture. As used herein, a controlled release formulation comprises a compound of the present invention (e.g., a protein (including homologues), a drug, an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a recipient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a compound of the present invention at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A pharmaceutically acceptable carrier which is capable of targeting is herein referred to as a “delivery vehicle.” Delivery vehicles of the present invention are capable of delivering a composition of the present invention to a target site in a patient. A “target site” refers to a site in a recipient to which one desires to deliver a composition. For example, a target site can be any cell which is targeted by direct injection or delivery using liposomes, viral vectors or other delivery vehicles, including ribozymes and antibodies. Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a recipient, thereby targeting and making use of a compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

One delivery vehicle of the present invention is a liposome. A liposome is capable of remaining stable in an animal for a sufficient amount of time to deliver a nucleic acid molecule or other compound to a preferred site in the recipient, typically an animal. A liposome, according to the present invention, comprises a lipid composition that is capable of delivering a nucleic acid molecule or other compound to a particular, or selected, site in a patient. A liposome according to the present invention comprises a lipid composition that is capable of fusing with the plasma membrane of the targeted cell to deliver a nucleic acid molecule or other compound into a cell. Suitable liposomes for use with the present invention include any liposome. Preferred liposomes of the present invention include those liposomes commonly used in, for example, gene delivery methods known to those of skill in the art. More preferred liposomes comprise liposomes having a polycationic lipid composition and/or liposomes having a cholesterol backbone conjugated to polyethylene glycol. Complexing a liposome with a nucleic acid molecule or other compound can be achieved using methods standard in the art.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxyiruses, adenoviruses, herpesviruses, lentiviruses, adeno-associated viruses and retroviruses.

Preferred methods of delivery of a gene to a plant cell have been described in detail above.

A composition which includes an compound identified according to the present methods can be delivered to a recipient by any suitable method. Selection of such a method will vary with the recipient, the type of compound being administered or delivered (i.e., protein, peptide, nucleic acid molecule, mimetic, or other type of compound), the mode of delivery (i.e., in vitro, in vivo, ex vivo) and the goal to be achieved by administration/delivery of the compound or composition. According to the present invention, an effective administration protocol (i.e., administering a composition in an effective manner) comprises suitable dose parameters and modes of administration that result in delivery of a composition to a desired site (i.e., to a desired cell) and/or in the desired regulatory event (e.g., inhibition of the biological activity of an AHL synthase and/or of quorum sensing of a population of microbes).

Administration routes include in vivo, in vitro and ex vivo routes. In vivo routes include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue for animal recipients, and transformation, particle bombardment, electroporation, microinjection, chemical treatment of cells, lipofection, adsorption, infection (e.g., Agrobacterium mediated transformation and virus mediated transformation) and protoplast fusion (protoplast transformation) for microbial and plant recipients. Preferred parenteral routes for animal administration can include, but are not limited to, subcutaneous, intradermal, intravenous, intramuscular and intraperitoneal routes. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of a compound. Ex vivo refers to performing part of the regulatory step outside of the recipient, such as by transfecting a population of cells removed from a recipient with a recombinant molecule comprising a nucleic acid sequence encoding a protein according to the present invention under conditions such that the recombinant molecule is subsequently expressed by the transfected cell, and returning the transfected cells to the recipient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

Another embodiment of the present invention relates to an antibody that selectively binds to an AHL synthase of the present invention and particularly, to a novel AHL synthase described herein, including the protein represented by SEQ ID NO:67 and homologues thereof. Such antibodies are useful for the identification and purification of AHL synthases, for example. In addition, such antibodies can be expressed in plants in order to sequester AHLs that are produced by infecting bacteria.

According to the present invention, the phrase “selectively binds to” refers to the ability of an antibody, antigen binding fragment or binding partner to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Another embodiment of the present invention relates to a computer for producing a three-dimensional model of a molecule or molecular structure, wherein the molecule or molecular structure comprises a three dimensional structure defined by atomic coordinates of an AHL synthase according to any one of Tables 2-5, or a three-dimensional model of a homologue of the molecule or molecular structure as described above. The computer comprises: (a) a computer-readable medium encoded with the atomic coordinates of the AHL synthase as described previously herein to create an electronic file; (b) a working memory for storing a graphical display software program for processing the electronic file; (c) a processor coupled to the working memory and to the computer-readable medium which is capable of representing the electronic file as the three dimensional model; and, (d) a display coupled to the processor for visualizing the three dimensional model. The three dimensional structure of the AHL synthase is displayed or can be displayed on the computer.

All publications and patents referenced herein are incorporated herein by reference in their entireties.

The following examples are provided for the purpose of illustration and are not intended to limit the scope of the present invention.

### EXAMPLES

### Example 1

This examples describes the crystallization, data collection and analysis of EsaI.

Overexpression and Purification

The gene encoding EsaI was subcloned into pET14b by PCR from the parent plasmid pSVB5-18, which is a pBluescriptSK+ derivative that carries the native esaI/esaR gene cluster (Beck von Bodman and Farrand, 1995). Primers used to amplify the EsaI coding sequence for subcloning into the NcoI/XhoI-digested pET14b vector, where the NcoI site reconstitutes the ATG initiation codon, are 5′-CTCTCGGAATCATATGCTTGAACTG-3′ (SEQ ID NO: 80) and 5′-CTCGTAGTAGAACCTCGAGTTATCAGACC-3′ (SEQ IDNO:81). Digestion of the PCR product with NcoI and XhoI allowed ligation of the EsaI coding sequence into the similarly digested pET14b vector. The final plasmid was verified by DNA sequencing.

EsaI was overexpressed in E. coli strain BL21 (DE3; Novagen) (Studier et al., 1990, Methods Enzymol., 185:60-89), grown in a fermentor in ampicillin-containing minimal media with lactose induction (0.2% w/v) as described previously (Hoffman et al., 1995). The cell pellet was stored at −80° C. The frozen cell paste (60 g) was thawed on ice, and resuspended in 200 ml of PBS (50 mM Na-K-phosphate and 0.3 M NaCl at pH 8.0) by vigorous pipetting and shaking. Cells were lysed by incubating in 0.75 mg/ml lysozyme, 100 mM benzamidine and 10 mM leupeptin on ice for 30 min, followed by sonication for 10 min at 30 watts, on a 50% duty cycle. Insoluble cellular debris was removed by centrifugation at 15,000 g. The supernatant was adjusted to pH=8.0 with NaOH and then incubated, with mixing, in a 1 ml bed volume of washed Ni-NTA resin (Qiagen) at 4° C. for 1 h. The EsaI-bound resin was washed three times with greater than ten bed volumes of 50 mM NaH₂PO₄ pH=8.0, 0.3 M NaCl, 10 mM imidazole, and packed into a column. The protein was eluted in an imidazole gradient of 10-250 mM imidazole, and fractions containing EsaI were pooled and dialyzed at 100-fold dilution three times into 20 mM HEPES pH=7.5, 0.3 M NaCl and 10 mM DTT. After dialysis to remove the

What is claimed is:

1. A method of structure-based identification of compounds which potentially bind to an AHL synthase, comprising:
a. obtaining atomic coordinates that define the three dimensional structure of an AHL synthase, said atomic coordinates being selected from the group consisting of:
i. atomic coordinates determined by X-ray diffraction of a crystalline EsaI or a crystalline LasI;
ii. atomic coordinates selected from the group consisting of:
(1) atomic coordinates represented in any one of Tables 2-5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe²⁸, Trp³⁴, Asp⁴⁵, ASp⁴⁸, Arg⁶⁸, Glu⁹⁷, or Arg¹⁰⁰ or to the following residues in SEQ ID NO:2: Arg²³, Phe²⁷, Trp³³, Asp⁴⁴, ASP⁴⁷, Arg⁷⁰, Glu¹⁰¹ or Arg¹⁰⁴; and
wherein said structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; and
(3) atomic coordinates in any one of Tables 2-5 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p43 so as to form a unit cell having approximate dimensions of a=b=66.40, c=47.33;
iv. atomic coordinates defining the three dimensional structure of EsaI molecules arranged in a crystalline manner in a space group p4₃ so as to form a unit cell having approximate dimensions of a=b=66.99, c=47.01;
v. atomic coordinates defining the three dimensional structure of LasI molecules arranged in a crystalline manner in a space group F23, so as to form a unit cell having approximate dimensions of a—b=c=154.90 Å; and
b. selecting candidate compounds for binding to said AHL synthase by performing structure based drug design with said structure of (a), wherein said step of selecting is performed in conjunction with computer modeling.

2. The method of claim 1, wherein said method further comprises:
c. selecting candidate compounds of (b) that inhibit the biological activity of an AHL synthase.

3. The method of claim 2, wherein said step (c) of selecting comprises:
i. contacting said candidate compound identified in step (b) with said AHL synthase; and
ii. measuring the enzymatic activity of said AHL synthase, as compared to in the absence of said candidate compound.

4. The method of claim 1, wherein said method further comprises:
c. selecting candidate compounds of (b) that inhibit the binding of an AHL synthase to its substrate.

5. The method of claim 4, wherein said step (c) of selecting comprises:
i. contacting said candidate compound identified in step (b) with said AHL synthase or a fragment thereof and a corresponding substrate or an AHL-synthase binding fragment thereof under conditions in which an AHL synthase-substrate complex can form in the absence of said candidate compound; and
ii. measuring the binding of said AHL synthase or fragment thereof to said substrate or fragment thereof, wherein a candidate inhibitor compound is selected when there is a decrease in the binding of the AHL synthase or fragment thereof to the substrate or fragment thereof, as compared to in the absence of said candidate inhibitor compound.

6. The method of claim 4, wherein said substrate is selected from the group consisting of S-adenosyl-L-methionine (SAM), an acylated acyl carrier protein (acyl-ACP), an acylated Coenzyme A molecule, and AHL-binding fragments thereof.

7. The method of claim 1, wherein said step of selecting comprises identifying candidate compounds for binding to the phosphopantetheine binding fold of said AHL synthase.

8. The method of claim 1, wherein said step of selecting comprises identifying candidate compounds for binding to the acyl chain binding region of said AHL synthase.

9. The method of claim 1, wherein said step of selecting comprises identifying candidate compounds for binding to the acyl-ACP binding site of said AHL synthase.

10. The method of claim 1, wherein said step of selecting comprises identifying candidate compounds for binding to the SAM binding site of said AHL synthase.

11. The method of claim 1, wherein said step of selecting comprises identifying candidate compounds for binding to the electrostatic cluster of said AHL synthase.

12. The method of claim 1, wherein said AHL synthase is a EsaI, and wherein said atomic coordinates are selected from the group consisting of:
i. atomic coordinates determined by X-ray diffraction of a crystalline EsaI;
ii. atomic coordinates selected from the group consisting of:
(1) atomic coordinates represented in any one of Tables 2-4;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe28, Trp³⁴, Asp⁴⁵, Asp⁴⁸, Arg⁶⁸, Glu⁹⁷, or Arg¹⁰⁰; and
wherein said structure has an amino acid sequence comprising at least three regions having detectable sequence homology with the following three regions in SEQ ID NO: 1: amino acid residues 19 through 56, amino acid residues 63-83, and amino acid residues 90-101; and
(3) atomic coordinates in any one of Tables 2-4 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p4₃ so as to form a unit cell having approximate dimensions of a=b=66.40, c=47.33; and
iv. atomic coordinates defining the three dimensional structure of EsaI molecules arranged in a crystalline manner in a space group p4₃ so as to form a unit cell having approximate dimensions of a—b=66.99, c=47.01.

13. The method of claim 12, wherein said step of selecting comprises selecting candidate compounds for binding to the electrostatic cluster of said AHL synthase comprising positions corresponding to amino acid positions S99, R68, R100, D45, and D48 of SEQ ID NO: 1.

14. The method of claim 12, wherein said step of selecting comprises selecting candidate compounds for binding to the SAM binding site of said AHL synthase comprising positions corresponding to amino acid positions 19 through 56 of SEQ ID NO: 1.

15. The method of claim 12, wherein said step of selecting comprises selecting candidate compounds for binding in a region comprising the acyl chain binding site, comprising positions corresponding to amino acid positions S98, F 123, M126, T 140, V 142, S143, M 146, I149, L150, S153, W155, I157, L176 or A178 of SEQ ID NO:1.

16. The method of claim 12, wherein said step of selecting comprises selecting candidate compounds for binding to the acyl chain binding site, comprising positions corresponding to amino acid positions S98, M126, T140, V142, M146, or L176 of SEQ ID NO:1.

17. The method of claim 1, wherein said AHL synthase is LasI, and wherein said atomic coordinates are selected from the group consisting of:
i. atomic coordinates determined by X-ray diffraction of a crystalline LasI;
ii. atomic coordinates selected from the group consisting of:
(1) atomic coordinates represented in Table 5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO:2: Arg²³, Phe²⁷, Trp³³, Asp⁴⁴, Asp⁴⁷, Arg⁷⁰, Glu¹⁰¹ or Arg¹⁰⁴; and
wherein said structure has an amino acid sequence comprising at least three regions having detectable sequence homology with the following three regions in SEQ ID NO:2: amino acid residues 18-55, 65-85 and 95-105; and
(3) atomic coordinates in Table 5 defining a portion of said AHL synthase, wherein the portion of said AHL synthase comprises sufficient structural information to perform step (b); and
iii. atomic coordinates defining the three dimensional structure of LasI molecules arranged in a crystalline manner in a space group F23, so as to form a unit cell having approximate dimensions of a=b=c=154.90 Å.

18. The method of claim 17, wherein said step of selecting comprises selecting candidate compounds for binding to the electrostatic cluster of said AHL synthase comprising positions corresponding to amino acid positions 8, 20, 23, 42, 47, 49, 53, 67, 100 or 101 of SEQ ID NO:82.

19. The method of claim 17, wherein said step of selecting comprises selecting candidate compounds for binding to the SAM binding site of said AHL synthase comprising positions corresponding to amino acid positions 26, 27, 30, 33, 66, 102, 104, 106, 114, 140, 141, 142, or 145 of SEQ ID NO:82.

20. The method of claim 17, wherein said step of selecting comprises selecting candidate compounds for binding in a region comprising the acyl chain binding site, comprising positions corresponding to amino acid positions 99, 100, 118, 122, 137, 139, 141, 145, 148, 149, 152, 154, 175, 181, 184, or 185 of SEQ ID NO:82.

21. The method of claim 17, wherein said step of selecting comprises selecting candidate compounds for binding to the ACP binding site, comprising positions corresponding to amino acid positions 147, 150, 151 or 180 of SEQ ID NO:82.

22. The method of claim 1, wherein said atomic coordinates are atomic coordinates represented in any one of Tables 2-5.

23. The method of claim 1, wherein said atomic coordinates are atomic coordinates represented in any one of Tables 2-4.

24. The method of claim 1, wherein said atomic coordinates are atomic coordinates represented in Table 5.

25. The method of claim 1, wherein said atomic coordinates are atomic coordinates defining the three dimensional structure of EsaI molecules arranged in a crystalline manner in a space group p4₃ so as to form a unit cell of dimensions a=b=66.40, c=47.33.

26. The method of claim 1, wherein said atomic coordinates are atomic coordinates defining the three dimensional structure of EsaI molecules arranged in a crystalline manner in a space group p4₃ so as to form a unit cell of dimensions a=b=66.99, c=47.01.

27. The method of claim 1, wherein said atomic coordinates are atomic coordinates defining the three dimensional structure of LasI molecules arranged in a crystalline manner in a space group F23, so as to form a unit cell of dimensions a=b=c=154.90 Å.

28. The method of claim 1, wherein said step of selecting comprises directed drug design.

29. The method of claim 1, wherein said step of selecting comprises random drug design.

30. The method of claim 1, wherein said step of selecting comprises grid-based drug design.

31. The method of claim 1, wherein said step of selecting comprises computational screening of one or more databases of chemical compounds.

32. A method to produce an AHL synthase homologue that catalyzes the synthesis of AHL compounds having antibacterial biological activity, comprising:
a. obtaining atomic coordinates that define the three dimensional structure of an AHL synthase, said atomic coordinates being selected from the group consisting of:
i. atomic coordinates determined by X-ray diffraction of a crystalline EsaI or a crystalline LasI;
ii. atomic coordinates selected from the group consisting of:
(1) atomic coordinates represented in any one of Tables 2-5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe²⁸, Trp³⁴, Asp⁴⁵, Asp⁴⁸, Arg⁶⁸, Glu⁹⁷, or Arg¹⁰⁰ or to the following residues in SEQ ID NO:2: Arg²³, Phe²⁷ Trp³³, Asp⁴⁴ Asp⁴⁷, Ar⁷⁰, Glu¹⁰¹ or Arg¹⁰⁴; and
wherein said structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; and
(3) atomic coordinates in any one of Tables 2-5 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p4₃ so as to form a unit cell having approximate dimensions of a=b=66.40, c=47.33;
iv. atomic coordinates defining the three dimensional structure of EsaI molecules arranged in a crystalline manner in a space group p4₃ so as to form a unit cell having approximate dimensions of a=b=66.99, c=47.01;
v. atomic coordinates defining the three dimensional structure of LasI molecules arranged in a crystalline manner in a space group F23, so as to form a unit cell having approximate dimensions of a—b=c=154.90 Å;
a. performing computer modeling with said atomic coordinates of (a) to identify at least one site in said AHL synthase structure that is predicted to modify the biological activity of said AHL synthase;
b. producing a candidate AHL synthase homologue that is modified in said at least one site identified in (b); and
c. determining whether said candidate AHL synthase homologue of (c) catalyzes the synthesis of AHL compounds having antibacterial biological activity.

33. A method to produce an AHL synthase homologue with modified biological activity as compared to a natural AHL synthase, comprising:
a. obtaining atomic coordinates that define the three dimensional structure of an AHL synthase, said atomic coordinates being selected from the group consisting of:
i. atomic coordinates determined by X-ray diffraction of a crystalline EsaI or a crystalline LasI;
ii. atomic coordinates selected from the group consisting of:
(1) atomic coordinates represented in any one of Tables 2-5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe²⁸, Trp³⁴, Asp⁴⁵, Asp⁴⁸, Arg⁶⁸, Glu⁹⁷, or Arg¹⁰⁰ or to the following residues in SEQ ID NO:2: Arg²³, Phe27, Trp³³, Asp⁴⁴, Asp⁴⁷, Arg⁷⁰, Glu¹⁰¹ or Arg¹⁰⁴; and
wherein said structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; and
(3) atomic coordinates in any one of Tables 2-5 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p4₃ so as to form a unit cell having approximate dimensions of a=b=66.40, c=47.33;
iv. atomic coordinates defining the three dimensional structure of EsaI molecules arranged in a crystalline manner in a space group p4₃ so as to form a unit cell having approximate dimensions of a=b=66.99, c=47.01;
V. atomic coordinates defining the three dimensional structure of LasI molecules arranged in a crystalline manner in a space group F23, so as to form a unit cell having approximate dimensions of a=b=c=154.90 Å;
a. using computer modeling of said atomic coordinates in (a) to identify at least one site in said AHL synthase structure that is predicted to contribute to the biological activity of said AHL synthase; and
b. modifying said at least one site in an AHL synthase protein to produce an AHL synthase homologue which is predicted to have modified biological activity as compared to a natural AHL synthase.

34. The method of claim 33, wherein said step of modifying in (c) comprises using computer modeling to produce a structure of an AHL synthase homologue on a computer.

35. The method of claim 33, wherein said step of modifying in (c) comprises making at least one modification in the amino acid sequence of said AHL synthase protein selected from the group consisting of an insertion, a deletion, a substitution and a derivatization of an amino acid residue in said amino acid sequence.

36. The method of claim 33, further comprising determining whether the AHL synthase homologue has modified AHL synthase biological activity.

37. A method to construct a three dimensional model of an AHL synthase, comprising:
a. obtaining atomic coordinates that define the three dimensional structure of a first AHL synthase, said atomic coordinates being selected from the group consisting of:
i. atomic coordinates determined by X-ray diffraction of a crystalline EsaI or a crystalline LasI;
ii. atomic coordinates selected from the group consisting of:
(1) atomic coordinates represented in any one of Tables 2-5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe²⁸, Trp³⁴, Asp⁴⁵, Asp⁴⁸, Arg⁶⁸, Glu⁹⁷, or Arg¹⁰⁰ or to the following residues in SEQ ID NO:2: Arg²³, Phe²⁷, Trp³³, Asp⁴⁴, ASP⁴⁷, Arg⁷⁰, Glu¹⁰¹ or Arg¹⁰⁴; and
wherein said structure has an amino acid sequence comprising at least three regions having detectable sequence homology with the following three regions in SEQ ID NO: 1: amino acid residues 19 through 56, amino acid residues 63-83, and amino acid residues 90-101; or with the following three regions in SEQ ID NO:2: amino acid residues 18-55, 65-85 and 95-105; and
(3) atomic coordinates in any one of Tables 2-5 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p⁴₃ so as to form a unit cell having approximate dimensions of a=b=66.40, c=47.33;
iv. atomic coordinates defining the three dimensional structure of EsaI molecules arranged in a crystalline manner in a space group p4₃ so as to form a unit cell having approximate dimensions of a=b=66.99, c=47.01;
v. atomic coordinates defining the three dimensional structure of LasI molecules arranged in a crystalline manner in a space group F23, so as to form a unit cell having approximate dimensions of a—b=c=154.90 Å; and
a. performing computer modeling with said atomic coordinates of (a) and an amino acid sequence of a second AHL synthase to construct a model of a three dimensional structure of said second AHL synthase.

38. The method of claim 37, wherein said step (b) is performed using molecular replacement.

39. The method of claim 37, wherein the second AHL synthase is a naturally occurring AHL synthase.

40. The method of claim 37, wherein the second AHL synthase is a homologue of the first AHL synthase.

41. The method of claim 37, wherein the second AHL synthase is from a microorganism listed in Table 1.

42. The method of claim 37, wherein the second AHL synthase is from a mycobacterium.

43. The method of claim 37, wherein the second AHL synthase is from Mycobacterium tuberculosis.

44. A crystal comprising an AHL synthase, wherein the crystal effectively diffracts X-rays for the determination of the atomic coordinates of the AHL synthase to a resolution of greater than 3.2 Å, and wherein said crystal has a space group p4₃ so as to form a unit cell having approximate dimensions of a=b=66.40, c=47.33.

45. A crystal comprising an AHL synthase, wherein the crystal effectively diffracts X-rays for the determination of the atomic coordinates of the AHL synthase to a resolution of greater than 3.2 Å, and wherein said crystal has a space group p4₃ so as to form a unit cell having approximate dimensions of a=b=66.99, c=47.01.

46. A crystal comprising an AHL synthase, wherein the crystal effectively diffracts X-rays for the determination of the atomic coordinates of the AHL synthase to a resolution of greater than 3.2 Å, and wherein said crystal has a space group F23, so as to form a unit cell having approximate dimensions of a=b=c=154.90 Å.

47. A therapeutic composition comprising a compound that inhibits the biological activity of an AHL synthase, said compound being identified by the method comprising:
a. obtaining atomic coordinates that define the three dimensional structure of an AHL synthase, said atomic coordinates being selected from the group consisting of:
i. atomic coordinates determined by X-ray diffraction of a crystalline EsaI or a crystalline LasI;
ii. atomic coordinates selected from the group consisting of:
(1) atomic coordinates represented in any one of Tables 2-5;
(2) atomic coordinates that define a three dimensional structure having an average root-mean-square deviation (RMSD) of equal to or less than about 1.7 Å over the backbone atoms in secondary structure elements of at least 50% of the residues in a three dimensional structure represented by said atomic coordinates of (1);
wherein said structure has an amino acid sequence comprising at least three of eight conserved amino acid residues corresponding to the following residues in SEQ ID NO: 1: Arg²⁴, Phe 2⁸, Trp³⁴, Asp⁴⁵, Asp⁴⁸, Arg⁶⁸, Glu⁹⁷, or Arg¹⁰⁰ or to the following residues in SEQ ID NO:2: Arg²³, Phe²⁷, Trp³³, Asp⁴⁴, Asp⁴⁷, Arg⁷⁰, Glu¹⁰¹ or Arg¹⁰⁴; and
wherein said structure has an amino acid sequence comprising at least three regions having detectable sequence homology with the following three regions in SEQ ID NO:1: amino acid residues 19 through 56, amino acid residues 63-83, and amino acid residues 90-101; or with the following three regions in SEQ ID NO:2: amino acid residues 18-55, 65-85 and 95-105; and
(3) atomic coordinates in any one of Tables 2-5 defining a portion of said AHL synthase, wherein the portion of said AHL synthase comprises sufficient structural information to perform step (b); and
iii. atomic coordinates defining the three dimensional structure of EsaI molecules arranged in a crystalline manner in a space group p4₃ so as to form a unit cell having approximate dimensions of a=b=66.40, c=47.33;
iv. atomic coordinates defining the three dimensional structure of EsaI molecules arranged in a crystalline manner in a space group p4₃ so as to form a unit cell having approximate dimensions of a=b=66.99, c=47.01;
v. atomic coordinates defining the three dimensional structure of LasI molecules arranged in a crystalline manner in a space group F23, so as to form a unit cell having approximate dimensions of a=b=c=154.90 Å; and
b. selecting candidate compounds for binding to said AHL synthase by performing structure based drug design with said structure of (a), wherein said step of selecting is performed in conjunction with computer modeling;
c. synthesizing said candidate compound selected in (b); and
d. further selecting candidate compounds that inhibit the biological activity of said AHL synthase.

48. A method to treat a disease or condition that can be regulated by modifying the biological activity of an AHL synthase or a compound produced by the enzymatic activity of said synthase, comprising administering to an organism with such a disease or condition the therapeutic composition of claim 47.

49. The method of claim 48, further comprising administering to said organism an antibacterial agent.

50. A transgenic plant or part of a plant comprising one or more cells that recombinantly express a protein compound identified by the method of claim 1.

51. A transgenic plant or part of a plant comprising one or more cells that recombinantly express a nucleic acid sequence encoding an AHL synthase homologue, wherein said AHL synthase homologue is identified by the method of claim 33.

52. An isolated protein comprising a mutant AHL synthase, wherein said protein comprises an amino acid sequence that differs from the amino acid sequence of a naturally occurring AHL synthase by at least one amino acid modification that results in a mutant AHL synthase that catalyzes the production of a different AHL product as compared to the naturally occurring AHL synthase.

53. The isolated protein of claim 52, wherein said protein comprises an amino acid sequence that differs from the amino acid sequence of a naturally occurring AHL synthase by at least one amino acid modification in the acyl chain binding region of said AHL synthase.

54. The isolated protein of claim 52, wherein said protein comprises a mutation in an amino acid residue corresponding to Thr¹⁴⁰ in SEQ ID NO: 1.

55. The isolated protein of claim 52, wherein said protein comprises a mutation in an amino acid residue corresponding to Ser⁹⁹ of SEQ ID NO: 1.

56. A transgenic plant or part of a plant comprising one or more cells that recombinantly express a nucleic acid sequence encoding a mutant AHL synthase of claim 52.

57. An isolated protein comprising a mutant EsaI protein, wherein said protein comprises an amino acid sequence that differs from SEQ ID NO:1 by at least one modification including at least one amino acid substitution selected from the group consisting of: a non-arginine amino acid residue at position 24, a non-phenyalanine amino acid residue at position 28, a non-tryptophan amino acid residue at position 34, a non-aspartate amino acid residue at position 45, a non-aspartate amino acid residue at position 48, a non-arginine amino acid residue at position 68, a non-glutamate amino acid residue at position 97, a non-serine amino acid residue at position 99, a non-arginine amino acid residue at position 100; and a non-threonine amino acid residue at position 140;
wherein said mutant EsaI protein has modified biological activity as compared to a wild-type EsaI protein.

58. The isolated mutant EsaI protein of claim 57, wherein said protein comprises an amino acid sequence that differs from SEQ ID NO: 1 by at least one modification including a substitution of a non-threonine amino acid residue at position 140.

59. The isolated mutant EsaI protein of claim 57, wherein said protein comprises an amino acid sequence that differs from SEQ ID NO: 1 by at least one modification including a substitution of a non-serine amino acid residue at position 99.

60. The isolated mutant EsaI protein of claim 57, wherein said protein comprises an amino acid sequence that differs from SEQ ID NO: 1 by an amino acid substitution selected from the group consisting of: an asparagine substituted for the aspartate at position 45, a glutamine substituted for the glutamate at position 97, an alanine substituted for the serine at position 99; a valine substituted for the threonine at position 140; and an alanine substituted for the threonine at position 140.

61. An isolated AHL synthase comprising an amino acid sequence selected from the group consisting of:
a. an amino acid sequence that is at least about 70% identical to an amino acid sequence selected from any of SEQ ID NOs:67 or SEQ ID NOs:83-100, wherein said amino acid sequence has AHL synthase activity; and
b. a fragment of an amino acid sequence of (a), wherein said fragment has AHL synthase activity.

62. The isolated AHL synthase of claim 61, wherein said amino acid sequence is selected from the group consisting of:
a. an amino acid sequence that is at least about 80% identical to an amino acid sequence selected from any of SEQ ID NOs:67 or SEQ ID NOs:83-100, wherein said amino acid sequence has AHL synthase activity; and
b. a fragment of an amino acid sequence of (a), wherein said fragment has AHL synthase activity.

63. The isolated AHL synthase of claim 61, wherein said amino acid sequence is selected from the group consisting of:
a. an amino acid sequence that is at least about 90% identical to an amino acid sequence selected from any of SEQ ID NOs:67 or SEQ ID NOs: 83-100, wherein said amino acid sequence has AHL synthase activity; and
b. a fragment of an amino acid sequence of (a), wherein said fragment has AHL synthase activity.

64. The isolated AHL synthase of claim 61, wherein said amino acid sequence is selected from any of SEQ ID NOs:67 or SEQ ID NOs:83-100, or a fragment thereof having AHL synthase activity.

65. The isolated AHL synthase of claim 61, wherein said amino acid sequence is less than 100% identical to an amino acid sequence selected from any of SEQ ID NOs:67 or SEQ ID NOs:83-100, wherein said amino acid sequence has AHL synthase activity.

66. The isolated AHL synthase of claim 61, wherein said amino acid sequence is less than 98% identical to an amino acid sequence selected from any of SEQ ID NOs:67 or SEQ ID NOs:83-100, wherein said amino acid sequence has AHL synthase activity.

67. The isolated AHL synthase of claim 61, wherein said AHL synthase is from a mycobacterium.

68. The isolated AHL synthase of claim 67, wherein said mycobacterium is selected from the group of Mycobacterium tuberculosis, Mycobacterium avium, Mycobacterium bovis, and Mycobacterium leprae.

69. An isolated nucleic acid molecule comprising a nucleic acid sequence selected from the group consisting of:
a. a nucleic acid sequence that encodes an amino acid sequence that is at least about 70% identical and less than 100% identical to an amino acid sequence selected from any of SEQ ID NOs:67 or SEQ ID NOs:83-100, wherein said amino acid sequence has AHL synthase activity;
b. a nucleic acid sequence encoding a fragment of said amino acid sequence of (a), wherein said fragment has AHL synthase activity;
c. a nucleic acid sequence that is a probe or primer that hybridizes under high stringency conditions to a nucleic acid sequence of (a) or (b); and
d. a nucleic acid sequence that is a complement of any of the nucleic acid sequences of (a)-(c).

70. The isolated nucleic acid molecule according to claim 69, wherein said nucleic acid sequence encodes an amino acid sequence that is at least about 80% identical and less than 100% identical to an amino acid sequence selected from any of SEQ ID NOs:67 or SEQ ID NOs:83-100, wherein said amino acid sequence has AHL synthase activity.

71. The isolated nucleic acid molecule according to claim 69, wherein said nucleic acid sequence encodes an amino acid sequence that is at least about 90% identical and less than 100% identical to an amino acid sequence selected from any of SEQ ID NOs:67 or SEQ ID NOs:83-100, wherein said amino acid sequence has AHL synthase activity.

72. A recombinant nucleic acid molecule comprising a nucleic acid molecule according to claim 69 that is operatively linked to at least one transcription control sequence.

73. A recombinant host cell transformed with a recombinant nucleic acid molecule of claim 72.

74. The recombinant host cell of claim 73, wherein said host cell is a prokaryotic cell.

75. The recombinant host cell of claim 73, wherein said host cell is a eukaryotic cell.

76. An isolated AHL synthase comprising an amino acid sequence selected from the group consisting of:
a. an amino acid sequence that is at least about 30% identical to SEQ ID NO:67, wherein said amino acid sequence comprises at least three amino acid residues corresponding to amino acid residues of SEQ ID NO:67 selected from: Arg⁹, Phe¹³, Phe¹⁹, Asp³², Asp³⁵, Arg⁵⁶, Glu⁸⁹ and Arg⁹², and wherein said amino acid sequence has AHL synthase activity; and
b. a fragment of an amino acid sequence of (a), wherein said fragment has AHL synthase activity.

77. A method of identifying a compound that regulates quorum sensing signal generation, comprising:
a. contacting an AHL synthase or biologically active fragment thereof with a putative regulatory compound, wherein said AHL synthase comprises an amino acid sequence that is at least about 70% identical to an amino acid sequence selected from any of SEQ ID NOs:67 or SEQ ID NOs:83-100, or a biologically active fragment thereof, wherein said amino acid sequence has AHL synthase activity;
b. detecting whether said putative regulatory compound increases or decreases a biological activity of said AHL synthase as compared to in the absence of contact with said compound;
wherein compounds that increases or decreases activity of the AHL synthase, as compared to in the absence of said compound, indicates that said putative regulatory compound is a regulator of said AHL synthase.

78. The method of claim 77, wherein said biological activity is selected from the group consisting of: the binding of said AHL synthase to a substrate, AHL enzymatic activity, synthesis of an AHL, quorum sensing signal generation in a population of microorganisms expressing said AHL synthase, and change in production of gene products dependent on the transcription factors that bind the AHL.

79. A method to inhibit quorum sensing signal generation in a population of microbial cells, comprising contacting a population of microbial cells that express an AHL synthase with an antagonist of said AHL synthase, wherein said antagonist decreases the biological activity of said AHL synthase, and wherein said AHL synthase comprises an amino acid sequence that is at least about 70% identical to an amino acid sequence selected from any of SEQ ID NOs:67 or SEQ ID NOs:83-100.

80. The method of claim 79, wherein said population of microbial cells infects a plant.

81. The method of claim 80, wherein said plant is transgenic for the expression of said antagonist of said AHL synthase.

82. The method of claim 79, wherein said population of microbial cells infects an animal.


SEQUENCE LISTING (100 sequences)

SEQ ID NO: 1 - protein, 210 aa, Erwinia stewartii
MLELFDVSYEELQTTRSEELYKLRKKTFSDRLGWEVICSQGMESDEFDGPGTRYILGICEGQLVCSVRFTSLDRPNMITHTFQHCFSDVTLPAYGTESSRFFVDKARARALLGEHYPISQVLFLAMVNWAQNNAYGNIYTIVSRAMLKILTRSGWQIKVIKEAFLTEKERIYLLTLPAGQDDKQQLGGDVVSRTGCPPVAVTTWPLTLPV

SEQ ID NO: 2 - protein, 201 aa, Pseudomonas aeruginosa
MIVQIGRREEFDKKLLGEMHKLRAQVFKERKGWDVSVIDEMEIDGYDALSPYYMLIQEDTPEAQVFGCWRILDTTGPYMLKNTFPELLHGKEAPCSPHIWELSRFAINSGQKGSLGFSDCTLEAMRALARYSLQNDIQTLVTVTTVGVEKMMIRAGLDVSRFGPHLKIGIERAVALRIELNAKTQIALYGGVLVEQRLAVS

SEQ ID NO: 3 - protein, 193 aa, Vibrio fischeri
MTIMIKKSDFLAIPSEEYKGILSLRYQVFKQRLEWDLVVENNLESDEYDNSNAEYIYACDDTENVSGCWRLLPTTGDYMLKSVFPELLGQQSAPKDPNIVELSRFAVGKNSSKINNSASEITMKLFEAIYKHAVSQGITEYVTVTSTAIERFLKRIKVPCHRIGDKEIHVLGDTKSVVLSMPINEQFKKAVLN

SEQ ID NO: 4 - protein, 196 aa, Pseudomonas aeruginosa
MIELLSESLEGLSAAMIAELGRYRHQVFIEKLGWDVVSTSRVRDQEFDQFDHPQTRYIVAMSRQGICGCARLLPTTDAYLLKDVFAYLCSETPPSDPSVWELSRYAASAADDPQLAMKIFWSSLQCAWYLGASSVVAVTTTAMERYFVRNGVILQRLGPPQKVKGETLVAISFPAYQERGLEMLLRYHPEWLAEPR

SEQ ID NO: 5 - protein, 207 aa, Aeromonas hydrophila
MLVFKGKLKEHPRWEVENELYRFRNRVFSDRLGWDVESHRGLEQDSFDTPDTHWVLIEDEEGLCGCIRLLSCAKDYMLPSIFPTALAGEAPPRSNDVWELTRLAIDAERAPRLGNGISELTCIIFREVYAFAKAQGIRELVAVVSLPVERIFRRLGLPIERLGHRQAVDLGAVRGVGIRFHLDERFARAVGQPLQGAYDEARELVTE

SEQ ID NO: 6 - protein, 207 aa, Aeromonas salmonicida
MLVFKGKLKEHPRWEVENELYRFRNRVFSDRLGWDVESHRGLEQDSFDTPDTHWVLIEDEEGLCGCIRLLSCAQDYMLPSIFPTALAGEAPPRSSDVWELTRLAIDANRAPRMGNGVSELTCVIFREVYAFARAKGIRELVAVVSLPVERIFRRLGLPIERLGHRQAVDLGAVRGVGIRFHLDERFARAVGHPMQGEYADARELVTE

SEQ ID NO: 7 - protein, 202 aa, Burkholderia ambifaria
MRTFVHEEGRLPHELAADLGRYRRRVFVEQLGWALPSANESFERDQFDRDDTVYVFARNAGGDVCGCARLLPTTRPYLLKSLFADLVAEGVPLPQSAAVWELSRFAATGDEGGPGNAEWAVRPMLAAVVECAAQLGARQLIGVTFASMERLFRRIGVHAHRAGPPKQVDGRLVVACWIDIDPQTFAALGIEPGRAARQAIAA

SEQ ID NO: 8 - protein, 202 aa, Burkholderia cepacia
MQTFVHEEGRLPHELAADLGRYRRRVFVEQLGWALPSANESFERDQFDRDDTVYVFARNADGDMCGCARLLPTTRPYLLKSLFADLVAEDMPLPQSAAVWELSRFAATDDEGGPGNAEWAVRPMLAAVVECAAQLGARQLIGVTFASMERLFRRIGIHAHRAGPPKQVDGRLVVACWIDIDPQTFAALGIEPGQAARQAIAA

SEQ ID NO: 9 - protein, 202 aa, Burkholderia cepacia
MRTFVHEEGRLPHELAADLGRYRRRVFVEQLGWALPSANESFERDQFDRDDTVYVFARNAGGDMCGCARLLPTTRPYLLKSLFADLIAEDMPLPQSAAVWELSRFAATDDEGGPGNAEWAVRPMLAAVVECAAQLGARQLIGVTFASMERLFRRIGIHAHRAGPPKQVDGRLVVACWIDIDPQTFAALGIEPGQAARQAIAA

SEQ ID NO: 10 - protein, 219 aa, Burkholderia cepacia
MLTLLSGRSADLNRETMYQLAKYRHKVFIQELGWTLPTDNGIEFDNFDHADTLYVIARDRNGEIVGCGRLLPTDEPYLLGDVFPTLMGDAALPHAPDVWELSRFAMSMPRGESLTAEESWQNTRAMMSEIVRVAHAHGANRLIAFSVLGNERLLKRMGVNVHRAAPPQMIEGKPTLPFWIEIDEQTRAALNLDGLERVGGVPPKTLRRPDASRALEQSV

SEQ ID NO: 11 - protein, 202 aa, Burkholderia cepacia
MQTFVHEEGRLPHELAADLGRYRRRVFVEQLGWALPSANVSFERDQFDRDDTVYVFARNADGDMCGCARLLPTTRPYLLKSLFADLIAEDMPLPQSAAVWELSRFAATDDEGGPGNAEWAVRPMLAAVVECAAQLGARQLIGVTFASMERLFRRIGIHAHRAGPPKQVDGRLVVACWIDIDPQTFAALGIEPGQAARQAIAA

SEQ ID NO: 12 - protein, 202 aa, Burkholderia cepacia
MQTFVHEEGRLPYELAADLGRYRRRVFVEQLGWALPSANEAFERDQFDRDDTVYVMARNAAGEMCGCARLLPTTQPYLLESLFADLVAQDVPLPKSAAVWELSRFAATADENGPGNAEWAVRPMLAAVVECAAQLGARQLIGVTFASMERLFRRIGVHAHRAGPPKQVDGRLVVACWIDIDPQTFAALGIEPGPAARQAIAA

SEQ ID NO: 13 - protein, 202 aa, Burkholderia multivorans
MQTFVHEGRQLPMPQATELARYRHRVFVEQLGWTLPSADEGIDRDAFDHDDTVYVVARDGSGEMCGCARLLPTTRPYLLETLFADLIAPDLPLPRSAAVWELSRFAASGADGGASGADWAVRPMLAAVVACAAERGARQLIGVTFASMERLFRRIGVHAHRAGPPKQVDGRLVVACWIDLDPQTFAALGIEPERIARPAIAA

SEQ ID NO: 14 - protein, 202 aa, Burkholderia multivorans
MQTFVHEGRQLPMPQATDVARYRHRVFVEQLGWTLPSADEGIDRDAFDHDDTVYVAARDGSGAMCGCARLLPTTRPYLLETLFADLIAPDLPLPRSAAVWELSRFAASGADGGASGADWAVRPMLAAVVACAAERGARQLIGVTFASMERLFRRIGVHAHRAGPPKQVDGRLVVACWIDLDPQTFAALGIEPERIARPAIAA

SEQ ID NO: 15 - protein, 202 aa, Burkholderia multivorans
MRTFVHEEGRLPSELAAELGRYRRRVFIEQLGWALPSANERFEHDQFDRDDTVYVFARDAGGDVCGCARLLPTTRPYLLESLFADLIAEDVALPQSAAVWELSRFAATGDEGGAGNADWAVRPMLAVVVECAAQLGARQLIGVTFASMERLFRRIGVHAHRAGPPKQVDGRLVVACWIDIDPQTFAALGITPGRAARQAIAA

SEQ ID NO: 16 - protein, 202 aa, Burkholderia multivorans
MQTFVHEGRQLPIAQATELARYRHRVFVEQLGWTLPSADEGIDRDAFDHDDTVYVVARDGSGAMCSCARLLPTTRPYLLETLFADLIAPDLPLPRSAAVWELSRFAASGADGGASGADWAVRPMLAAVVACAAERGARQLIGVTFASMERLFRRIGVHAHRAGPPKQVDGRLVVACWIDLDPQTFAALGIEPERIARPAIAA

SEQ ID NO: 17 - protein, 202 aa, Burkholderia multivorans
MQTFVHEGRQLPIAQATELARYRHRVFVEQLGWTLPSADEGIDRDAFDHDDTVYVVARDGSGAMCGCARLLPTTRPYLLETLFADLIAPDLPLPRSAAVWELSRFAASGADGGASGADWAVRPMLAAVVACAAERGARQLIGVTFASKERLFRRIGVHAHRAGPPKQVDGRLVVACWIDLDPQTFAALGIEPERIARPAIAA

SEQ ID NO: 18 - protein, 202 aa, Burkholderia vietnamiensis; identical to SEQ ID NO: 15

SEQ ID NO: 19 - protein, 202 aa, Burkholderia stabilis
MRTFVHEEGRLPHELAADIGRYRRRVFVEQLGWALPSANESFERDQFDRDDTVYVFARNADGDMCGCARLLPTTRPYLLGSLFADLIAEDMPLPQSAAVWELSRFAATDDESGSGNAEWAVRPMLAAVVECAAQLGARQLIGVTFASMERLFRRIGIHAHRAGPPKQVDGRLVVACWIDIDPQTFAALGIEPGQASRQAIAA

SEQ ID NO: 20 - protein, 216 aa, Erwinia carotovora
MLEIFDVNHTLLSETKSGELFTLRKETFKDRLNWAVQCTDGMEFDQYDNNNTTYLFGIKDNTVICSLRFIETKYPNMITGTFFPYFKEINIPEGNYLESSRFFVDKSRAKDILGNEYPISSMLFLSMINYSKDKGYDGIYTIVSHPMLTILKRSGWGIRVVEQGLSEKEERVYLVFLPVDDENQEALARRINRSGTFMSNELKQWPLRVPAAIAQA

SEQ ID NO: 21 - protein, 216 aa, Erwinia carotovora
MLEIFDVNHTLLSETKSEELFTLRKETFKDRLNWAVQCTDGMEFDQYDNNNTTYLFGIKDNTVICSLRFIETKYPNMITGTFFPYFKEINIPEGNYLESSRFFVDKSRAKDILGNEYPISSMLFLSMINYSKDKGYDGIYTIVSHPMLTILKRSGWGIRVVEQGLSEKEERVYLVFLPVDDENQEALARRINRSGTFMSNELKQWPLRVPAAIAQA

SEQ ID NO: 22 - protein, 216 aa, Erwinia carotovora
MLEIFDVNHTLLSETKSEELFTLRKETFKDRLNWAVQCTDGMEFDQYDNNNTTYLFGIKDNTVICSLRFIETKYPNMITGTFSPYFKEINIPEGNYLESTRFFVDKSRAKEILGNEYPISSMLFLSMINYSRDKGYDGIYTIVSHPMLTILKRSGWGISVVEQGLSEKKERVYLVFLPVDDQNQDALARRINRSGTFMSNDLKQWPLRLPPAIVQA

SEQ ID NO: 23 - protein, 217 aa, Erwinia carotovora
MLEIFDVSYTLLSEKKSEELFTLRKETFKDRLNWAVKCINGMEFDQYDDDNATYLFGVEGDQVICSSRLIETKYPNMITGTFFPYFEKIDIPEGKYIESSRFFVDKARSKTILGNSYPVSTMFFLATVNYSKSKGYDGVYTIVSHPMLTILKRSGWKISIVEQGMSEKHERVYLLFLPVDNESQDVLVRRINHNQEFVESKLREWPLSFEPMTEPVG

SEQ ID NO: 24 - protein, 216 aa, Erwinia carotovora
MLEIFDVNHTLLSETKSEELFTLRKETFKDRLNWAVQCTDGMEFDQYDNNNTTYLFGIKDNTVICSLRFIETKYPNMITGTFFPYFKEINIPEGNYLESSRFFVDKSRAKDILGNEYPISSMLFLSMINYSRDKGYDGIYTIVSHPMLTILKRSGWGIRVVEQGLSEKEERVYLVFLPVDDENQEALARRINRSGTFMSNELKQWPLKGPAAIAQA

SEQ ID NO: 25 - protein, 212 aa, Erwinia chrysanthemi
MLEIFDVSFSLMSNNKLDEVFALRKGTFKDRLDWTVNCINGMEFDEYDNEHTTYLLGVKEGKIICSVRFIEMKYPNMITGTFFSYFDGLNIPEGNYIESSRFFVDRDRVRNLIGTRNPACLTLFLAMINYARKYHYDGILTIVSHPMLTLLKRSGWRISIIQQGLSEKQEKIYLLHLPTDDESRYALIERITRITNAESEQLTTLPLLVPLA

SEQ ID NO: 26 - protein, 212 aa, Erwinia chrysanthemi
MLEIFDVSFSLMSNNKLDEVFTLRKDTFKDRLDWAVNCINGMEFDEYDNEHTTYLLGVKEGKVICSVRFIEIKYPNMITGTFYSYFDNLKIPEGNYIESSRFFVDRDRVRNLIGTRNPACVTLFLAMINYARKYHYDGILTIVSHPMLTLLKRSGWRISIIQQGLSEKQERIYLLHLPTDDDSRHALIERITQMTQAESEQLKTLPLLVPLA

SEQ ID NO: 27 - protein, 216 aa, Pantoea agglomerans
MLEIFDVSYNDLTERRSEDLYKLRKITFKDRLDWAVNCSNDMEFDEFDNSGTRYMLGIYDNQLVCSVRFIDLRLPNMITHTFQHLFGDVKLPEGDYIESSRFFVDKNRAKALLGSRYPISYVLFLSMINYARHHGHTGIYTIVSRAMLTIAKRSGWEIEVIKEGFVSENEPIYLLRLPIDCHNQHLLAKRIRDQSESNIAALCQWPMSLTVTPEQV

SEQ ID NO: 28 - protein, 216 aa, Enterobacter agglomerans
MLEIFDVSYNDLTERRSEDLYKLRKITFKDRLDWAVNCSNDMEFDEFDNSGTRYMLGIYDNQLVCSVRFIDLRLPNMITHTFQHLFGDVKLPEGDYIDSSRFFVDKNRAKALLGSRYPISYVLFLSMINYARHHGHTGIYTIVSRAMLTIAKRSGWEIEVIKEGFVSENEPIYLLRLPIDCHNQHLLAKRIRDQSESNIAALCQCPMSLTVTPEQV

SEQ ID NO: 29 - protein, 201 aa, Pseudomonas aeruginosa
MIELLSESLEGLSAAMIAELGRYRHQVFIEKLGWDVVSTSRVRDQEFDQFDHPQTRYIVAMSRQGICGCARLLPTTDAYLLKDVFAYLCSETPPSDPSVWELSRYAASAADDPQLAMKIFWSSLQCAWYLGASSVVAVTTTAMERYFVRNGVILQRLGPPQKVKGETLVAISFPAYQERGLEMLLRYHPEWLQGVPLSMAV

SEQ ID NO: 30 - protein, 201 aa, Pseudomonas aeruginosa
MIELLSESLEGLSAAMIAELGRYRHQVFIEKLGWDVVSTSRVRDQEFDQFDHPQTRYIVAMGRQGICGCARLLPTTDAYLLKEVFAYLCSETPPSDPSVWELSRYAASAADDPQLAMKIFWSSLQCAWYLGASSVVAVTTTAMERYFVRNGVILQRLGPPQKVKGETLVAISFPAYQERGLEMLLRYHPEWLQGVPLSMAV

SEQ ID NO: 31 - protein, 200 aa, Pseudomonas aeruginosa
MIEFLSESLEGLSAAMIAELGRYRHQVFIEKLGWDVVSTSRVRDQEFDQFDHPQTRYIVAMGRQGICGCARLLPTCDAYLLKEVFAYLCSETPPSDPSVWELSRYAASAADDPQLAMKIFWSSLQCAWYLGASSVVAVTTTAMERYFVRNGVILQRLGPPQKVKGETLVAISFPAYQERGLEMLLRYHPEWLQRTLSMAV

SEQ ID NO: 32 - protein, 200 aa, Pseudomonas aeruginosa
MIEFLSESLEGLSAAMIAELGRYRHQVFIEKLGWDVVSTSRVRDQEFDQFDHPQTRYIVAMGRQGICGCARLLPTTDAYLLKEVFAYLCSETPPSDPSVWELSRYAASAADDPQLAMKIFWSSLQCAWYLGASSVVAVTTTAMERYFVRNGVILQRLGPPQKVKGETLVAISFPAYQERGLEMLLRYHPEWLQRTLSMAV

SEQ ID NO: 33 - protein, 257 aa, Pseudomonas corrugata
MNYSSCIHHFSGVSTRIASYSKIPARTLQQILSIRKIAFIDRKKWDIESYQGSDYEWDEYDDPDAVYIYTHVNERVTGCVRLRPSNKPTLMSGALSFILPTDNTRPRSHDCWEATRFALSTDKTAAGELTQANIDVRTAALFLSMIKFAQLQNIETYEIIVDTLMEKILKRSGWRLDRRNTALGSKGETIIYGTLPCTLETYKEILRKNAIQTITAYEQFLMANRSSDISHKPFDIDQTLTDRNIPAKKHMHTELHL

SEQ ID NO: 34 - protein, 196 aa, Pseudomonas aureofaciens
MHMEEHTLNQMSDELKLMLGRFRHEQFVEKLGWRLPAHPSQAGCEWDQYDTEHARYLLAFNEDRAIVGCARLIPTTFPNLLEGVFGHTCAGAPPKHPAIWEMTRFTTREPQLAMPLFWRSLKTASLAGADAIVGIVNSTMERYYKINGVHYERLGPVTVHQNEKILAIKLSAHREHHRSAVAPSAFMSDTLLRETA

SEQ ID NO: 35 - protein, 226 aa, Pseudomonas fluorescens
MESIEFHALDYSPTPHAWVADLYGLRKEVFADRLNWKVNVRDDIEFDEYDNERTTYLVGTWKNVPLAGLRLINTLDPYMVEGPFRGFFSCEPPKQALMAESSRFFVDKTRSRQLGLAHLPLTEMLLLCMHNHAAHSGLESIITVVSNAMGRIVRNAGWHYEILDVGEAAPGEKVLLLDMPVSDANRQRLLSNIARKCPLSSTRLDTWPQRLNPLETALCEPQRISA

SEQ ID NO: 36 - protein, 196 aa, Pseudomonas fluorescens
MHMEEHALSAMDDELKLMLGRFRHEQFVEKLGWRLPIPPHQAGYEWDQYDTEHARYLLAFNEHRSIVGCARLIPTTFPNLLEGVFSHACAGAPPRHPAIWEMTRFTTREPQLAMPLFWKTLKTASLAGADAIVGIVNSTMERYYKINGVKYERLGSVIDHQNEKILAIKLSAHREHHRGARLPSGFTSEALLEETA

SEQ ID NO: 37 - protein, 191 aa, Pseudomonas fluorescens
MKYLIDKRENISPRYLEGMHKLRASIFKDKKGWDVSIIADMEIDGYDALAPTYMLLIDDINENKVAGCWRILPTTGPYMLKDTFPNLLTTKKPPRAANIWELSRFAISASERGGFGFSNTAMKAIGHLIRHAHSQHVEKLITVTSVGVEKMLMKAGLELVRLGPPLTIGVERAIAVEVNLSNKTLDAVNAI

SEQ ID NO: 38 - protein, 219 aa, Pseudomonas fluorescens
MITVISRHESQLSPALRDDLGRYRHAVFIEQLGWQLPSSNHQPGHELDQFDHADTRYTLALDSEEKIHGCARLLPTTQPYLLSEVFGFLCDRPLPRQDDTWEISRFAASALEHGKLPMRVFWHTLHTAWTLGANSVVAVTTPALERYFLRHGVALSRLGQPQRVNRDHLLALDFPAYQKNGRAALYTQSAAVASLNQAFLRGNPPPTRGGPPTGQALRE

SEQ ID NO: 39 - protein, 196 aa, Pseudomonas chlororaphis
MHMEEHTLNGMSDELKLMLGRFRHEQFVEKLGWRLPAHPSQPGCEWDQYDTEHARYLLAFNEDCAIVGCARLIPTTFPNLLEGVFGHTCAGAPPKHPAIWEMTRFTTREPQLAMPLFWRSLKTASLAGADAIVGIVNSTMERYYKINGVHYERLGPVTVHQNEKILAIKLSAHREHHRGAAAPSAFMSDTLLKEIA

SEQ ID NO: 40 - protein, 226 aa, Pseudomonas syringae tabaci
MSSGFEFQLASYTTMPVTLLETLYSMRKKIFSDRLEWKVRVSHAFEFDEYDNAATTYLVGSWNGVPLAGLRLINTCDPYMLEGPFRSFFDCPAPKNAAMAESSRFFVDTARARSLGILHAPLTEMLLFSMHNHAALSGLQSIITVVSKAMARIVRKSGWEHHVLSTGEASPGETVLLLEMPVTADNHQRLLGNIALRQPVTDDLLRWPIALGVSGSAPQACMHSAA

SEQ ID NO: 41 - protein, 226 aa, Pseudomonas syringae syringae
MSSGFEFQVASYSKVPVTLLETLYALRKKIFSDRLEWKVRVSQAFEFDDYDSAAATYLIGSWNGVPLAGLRLINTCDPYMLDGPFRSFFDYPAPRNAGMAESSRFFVDTERARSLGILHAPLTEMLLFSMHNHAASAGLESIITVVSKAMARIVRKSGWEHRVLATGEASPGETVLLLDMPVNADNHQRLLGTIALRQPVTDDLLRWPIPLDASASLQRARMDSAA

SEQ ID NO: 42 - protein, 244 aa, Pseudomonas syringae maculicola
MSSGFEFQAASYVHMPVELLESLYSMRKNIFSDRLEWNVRVSDTFEFDEYDNADATYLVGSWNGIPLAGLRLINTCDSYMLEGPFRSFFDYQPPRNVRVVESSRFFVDTIRARSLGIAHASLTGMLLFALHNHVASSGLDSVITVVSKAMARIVRKAGWVYRVLATGEATPGETVLLLEMPVTADNHRRLLDNIALRQRVTDDLLRWPIALGPSGCAQRACVSDHAHLDGLVNGLLCSADLEFA

SEQ ID NO: 43 - protein, 204 aa, Ralstonia solanacearum
MRTFVHGGGRLPEGIDAALAHYRHQVFVGRLGWQLPMADGTFERDQYDRDDTVYVVARDEGGTICGCARLLPTTRPYLLKDVFASLLMHGMPPPESPEVWELSRFAARSGAPCPRSGRADWAVRPMLASVVQCAAQRGARRLIGATFVSMVRLFRRIGVRAHRAGPVRCIGGRPVVACWIDIDASTCAALGIPSASAAPGPVLQ

SEQ ID NO: 44 - protein, 212 aa, Rhizobium etli
MLRILTKDMLETDRRAFDEMFRARAAVFRDRLGWQVDVRDQWERDRYDEAEDPVYLVTQQPSGTLTGSLRLLPTTGATMLKSEFRHFFDQPIDVDSPTTWECTRFCLHPHAGDMKQSRAVATELLSGLCDLALDTGIENIVGVYDVAMVAVYRRIGWRPTPLARSRPEIGKLYVGLWDVTADNCRTLRANLSRLLEQASPYPARVLVDGGMR

SEQ ID NO: 45 - protein, 221 aa, Rhizobium leguminosarum
MFVIIQAHEYQKYAAVLDQMFRLRKKVFADTLCWDVPVIGPYERDSYDSLAPAYLVWCNDSRTRLYGGMRLMPTTGPTLLYDVFRETFPDAADLIAPGIWEGTRMCIDEEAIAKDFPEIDAGRAFSMMLLALCECALDHGIHTMISNYEPYLKRVYKRAGAEVEELGRADGYGKYPVCCGAFEVSDRVLRKMRAALGLTLPLYVRHVPARSVVTQFLEMAA

SEQ ID NO: 46 - protein, 221 aa, Rhodobacter sphaeroides; identical to SEQ ID NO: 45

SEQ ID NO: 47 - protein, 234 aa, Serratia sp.
MIDFFDLDYDSLSQKRSAELFSLRKKTFKDRLNWRVSCEQNMEFDVYDNKNTTYIFGVYEGSIICSLRFIETRFPNMIIDTFKPYFTQLHLPEGNHIEASRLFIDKERIRALHLQQHPISLLLFLSMINYARSLGYEGIYAIVSHPMLIIFQRSGWQVSIVEKGLSEKHQNIYLIHMPVDEHNQHLLIKHINKKSPLLNNTLNAWPLSFCVRENRSDQFQLDPKPYGMFGIGNT

SEQ ID NO: 48 - protein, 200 aa, Serratia liquefaciens
MIELFDVDYNLLPDNRSKELFSLRKKTFKDRLDWLVNCENNMEFDEYDNRHATYIFGTYQNHVICSLRFIETKYPNMISDGVFDTYFNDIKLPDGNYVEASRLFIDKARIQALQLHQAPISAMLFLSMINYARNCGYEGIYAIISHPMRIIFQRSGWHISVVKTGCSEKNKNIYLIYMPIDDANRNRLLARINQHATKMG

SEQ ID NO: 49 - protein, 212 aa, Agrobacterium tumefaciens
MLILTVSPDQYQHQNSYLKQMHRLRAEVFGNRLKWDVAIEDGGERDQYDELSPTYILATFGGQRVVGCARLLAPSGPTMLERTFPQLLATGSLSATTAMIETSRFCVDTTLPTGRAGRQLHLATLTMFAGIIEWSMANGYDEIVTATDLRFERILKRAGWPMTRLGEPVAIGNTVAVAGHLPADRKSFERVCPPGYRSIIADDNGRPLRSAA

SEQ ID NO: 50 - protein, 211 aa, Agrobacterium tumefaciens
MRILTVSPDQYERYRSFLKQMHRLRATVFGGRLEWDVSIIAGEERDQYDNFKPSYLLAITDSGRVAGCVRLLPACGPTMLEQTFSQLLEMGSLAAHSGMVESSRFCVDTSLVSRRDASQLHLATLTLFAGIIEWSMASGYTEIVTATDLRFERILKRAGWPMRRLGEPTAIGNTIAIAGRLPADRASFGQVCPPGYYSIPRIDVAAIRSAA

SEQ ID NO: 51 - protein, 211 aa, Plasmid pTiC58
MRILTVSPDQYERYRSFLKQMHRLRATVFGGRLEWDVSIIAGEERDQYDNFKPSYLLAITDSGRVAGCVRLLPACGPTMLEQTFSQLLEMGSLAAHSGMVESSRFCVDTSLVSRRDASQLHLATLTLFAGIIEWSMASGYTEIVTATDLRFERILKRAGWPMRRLGEPTAIGNTIAIAGRLPADRASFEQVCPPGYYSIPRIDVAAIRSAA

SEQ ID NO: 52 - protein, 191 aa, Rhizobium sp.
MQILAISKPRNIEEAQLLRSHHELRARVFSDRLGWEVNVVGGCESDTFDDLQPTYILAVSSNDRVVGCARLLPALGPTMVANVFPSLLSAGHLNAHSSMVESSRFCVDTFLAESRGDGSIHEATLTMFAGIIEWSVANRYTEIVTVTDLRFERILARVGWPLQRIGEPRPIGATVAVAGTLPAKADTFMRL

SEQ ID NO: 53 - protein, 208 aa, Rhizobium rhizogenes
MQILAISKPRNIEEAQLLRSHHELRARVFSDRLGWEVNVVGGCESDTFDDLQPTYILAVSSNDRVVGCARLLPALGPTMVANVFPSLLSAGHLNAHSSMVESSRFCVDTFLAESRGDGSIHEATLTMFAGIIEWSVANRYTEIVTVTDLRFERILARVGWPLQRIGEPRPIGATVAVAGTLPAKADTFMRLRPANYRSQIISTFGQSA

SEQ ID NO: 54 - protein, 213 aa, mesorhizobium loti
MIELIAPGWYGAFADELHEMHRLRYRVFKERLDWNVRTTGGFEIDSFDSLKPHYLVLRDSAGRVRGGVRLLPSTGPTMLRDVFSRLLEGRAAPEEPSVWESSRFALDLPPSAPKDSGSIAVATYELLAGMIEFGLSRLLTHIVTVTDLRMERILRRAGWPLDRIGPPQTIGTTCAVAGCLDVSEESLAAVRHNGGLGGPVLWAGALHGRLTWL

SEQ ID NO: 55 - protein, 226 aa, mesorhizobium loti
MAGTQPARRRRMIQLITPGLYSEFAGELKEMHGLRYRVFKERLDWEVQTGGEMETDTFDDLKPVYLLLKGSDWRIRGCVRLLPTTGPTMLRDTFPALLGEAVAPASPDIWESSRFALDLPPSTPKAAGGLAQATYELFAGMIEFGLANNLTRIVTVTDTRMERILRLATWPLSRIGKPQPVGKTEAVAGFLEISHASLLRIRWRGRLNGPVLWRPILGLPHGPCGS

SEQ ID NO: 56 - protein, 202 aa, mesorhizobium loti
MVRIHLVNWDNRKHYRKVLERYFRIRYEIYVKQRRWRAVARPINIEIDAFDNEHTLYVLALDTNGKIVGGSRLVPTLEPHLMSEVFPVLAGGRPPRAAEIFEWTRFFVVPSLRTQGAPSPIAGFVLCGLLETAQRLGIRQISVVCETFWPKRLRALGWTLTELGDVLEHPDGDIIALLIDVTPEAVEQTRRAYGISGVLLAE

SEQ ID NO: 57 - protein, 212 aa, mesorhizobium loti
MLFCLTTQELMERPDLWEAVHRLRYQIFVEEMGWEDLRRPDGFEVDQFDHDEAVHQIVLRGNEVAGYQRMLPTTRPHLLTEVLTDLSEGTPPSGPNIWELTRYAVAPGFRDGRRGVSTVGTELIAGFVEWGLKRGVDKVIIEFEPMWVLRALQLHFLATPLGYQRTYGNQQVVATLLSFNEHTLDVVRSRRNHHAPVLARGYPDMFGQRRAS

SEQ ID NO: 58 - protein, 193 aa, Vibrio fischeri
  misc_feature (72)..(72): X = any amino acid
MTIMIKKSDFLAIPSEEYKGILSLRYQVFKQRLEWDLVVENNLESDEYDNSNAEYIYACDDTENVSGCWRLXPTTGDYMLKSVFPELLGQQSAPKDPNIVELSRFAVGKNSSKINNSASEITMKLFEAIYKHAVSQGITEYVTVTSTAIERFLKRIKVPCHRIGDKEIHVLGDTKSVVLSMPINEQFKKAVLN

SEQ ID NO: 59 - protein, 193 aa, Vibrio fischeri
MAVMIKKSDFLGIPSEEYRGILSLRYQVFKRRLEWDLVSEDNLESDEYDNSNAEYIYACDDAEEVNGCWRLLPTTGDYMLKTVFPELLGDQVAPRDPNIVELSRFAVGKNSSKINNSASEITMKLFQAIYKHAVSQGITEYVTVTSIAIERFLKRIKVPCHRIGDKEIHLLGNTRSVVLSMPINDQFRKAVSN

SEQ ID NO: 60 - protein, 193 aa, Vibrio anguillarum
MTISIYSHTFQSVPQADYVSLLKLRYKVFSQRLQWELKTNRGMETDEYDVPEAHYLYAKEEQGHLVGCWRILPTTSRYMLKDTFSELLGVQQAPKAKEIYELSRFAVDKDHSAQLGGVSNVTLQMFQSLYHHAQQYHINAYVTVTSASVEKLIKRMGIPCERLGDKKVHLLGSTRSVALHIPMNEAYRASVNA

SEQ ID NO: 61 - protein, 214 aa, Yersinia enterocolitica
MLKLFNVNFNNMPERKLDEIFSLRKITFKDRLDWKVTCIDGKESDQYDDENTNYILGTIDDTIVCSVRFIDMKYPTMITGPFAPYFSDVSLPIDGFIESSRFFVEKALARDMVGNNSSLSTILFLAMVNYARDRGHKGILTVVSRGMFILLKRSGWNITVLNQGESEKNEVIYLLHLGIDNDSQQQLINKILRVHQVEPKTLETWPIIVPGIIK

SEQ ID NO: 62 - protein, 214 aa, Yersinia enterocolitica
MLKLFNVNFNNMPERKLDEIFSLREITFKDRLDWKVTCIDGKESDQYDDENTNYILGTIDDTIVCSVRFIDMKYPTMITGPFAPYFSDVSLPIDGFIESSRFFVEKALARDMVGNNSSLSTILFLAMVNYARDRGHKGILTVVSRGMFILLKRSGWNITVLNQGESEKNEVIYLLHLGIDNDSQQQLINKILRVHQVEPKTLETWPIIVPGIIK

SEQ ID NO: 63 - protein, 214 aa, Yersinia pestis
MLKVFNVNFDRMSENKLDEIFTLRKITFKDRLDWKVTCIDGKESDQYDDENTNYLLGTIDDTLVCSVRFVEMQYPTMITGPFAPYFRDLDLPIDGFIESSRFFVEKALARDKLGNNGSLSAILFLSMVNYARNRGYKGILTVVSRGMYTILKRSGWGITVINQGESEKNEVIYLLHLSIDSNSQQQLIRKIQRVHNIDTHTLASWPLVVPSMTK

SEQ ID NO: 64 - protein, 216 aa, Yersinia pseudotuberculosis
MLEIFDVRYDELTDIRSEDLYKLRKKTFKDRLNWEVNCSNGMEFDEYDNSDTRYLLGIYQGQLICSVRFIELHLPNMITHTFNALFDDVALPKRGYIESSRFFVDKTRAKLLFGNHYPISYLFFLSIINYSRHNGYTGIYTIVSRAMLTILKRSGWQVEVIKEAHITEKERIYLLHLPIDRDNQARLLLQVNQRLQDPCSVLSTWPISLPVMPESA

SEQ ID NO: 65 - protein, 214 aa, Yersinia pseudotuberculosis
MLKVFNVNFDRMSENKLDEIFTLRKITFKDRLDWKVTCIDGKESDQYDDENTNYLLGTIDDTLVCSVRFVEMQYPTMITGPFAPYFRDLDLPIDGFIESSRFFVEKALARDKLGNNGSLSAILFLSMVNYARNCGYKGILTVVSRGMYTILKRSGWGITVINQGESEKNEVIYLLHLSIDSNSQQQLIRKIQRVHNIDTHTLESWPLVVPSMTK

SEQ ID NO: 66 - protein, 216 aa, Yersinia ruckeri
MLEIFDVSYEELMDMRSDDLYRLRKKTFKDRLQWAVNCSNDMEFDEYDNPNTRYLLGIYGNQLICSVRFIELHRPNMITHTFNAQFDDIILPEGNYIESSRFFVDKSGAKTLLGNRYPISYVLFLAVINYTRHHKHTGIYTIVSRAMLTILKRSGWQFDVIKEAFVSEKERIYLLRLPVDKHNQALLASQVNQVLQGSDSALLAWPISLPVIPELV

SEQ ID NO: 67 - protein, 246 aa, Mycobacterium tuberculosis
MVEAAQRLRYDVFSTTPGFALPAAADTRRDGDRFDEYCDHLLVRDDDTGELVGCYRMLAPAGAIAAGGLYTATEFDVCAFDPLRPSLVEMGRAVVREGHRNGGVVLLMWAGILAYLDRYGYDYVTGCVSVPIGGDGETPGSRLRGVRDFILNRHAAPPQCQVYPYRPVRVDGRSLDDILPPPRPAVPPLMRGYLRLGARACGEPAHDPDFGVGDFCLLLDKDHADTRYLRRLRSVAAASEMVNDAR

SEQ ID NO: 68 - protein, 281 aa, Mycobacterium tuberculosis
MSIASVLIPSDKPHGVATGSSTGPRYSLLLSTDPSMVEAAQRLRYDVFSTTPGFALPAAADTRRDGDRFDEYCDHLLVRDDDTGELVGCYRMLAPAGAIAAGGLYTATEFDVCAFDPLRPSLVEMGRAVVREGHRNGGVVLLMWAGILAYLDRYGYDYVTGCVSVPIGGDGETPGSRLRGVRDFILNRHAAPPQCQVYPYRPVRVDGRSLDDILPPPRPAVPPLMRGYLRLGARACGEPAHDPDFGVGDFCLLLDKDHADTRYLRRLRSVAAASEMVNDAR

SEQ ID NO: 69 - protein, 256 aa, Streptomyces coelicolor
MTGVLTADRPPKPAAPRRYTVALARDEDDVRAAQRLRHDVFAGEMGALLASPQPGHDVDAFDAYCDHLLVREETTGQVVGTYRLLPPERAAVAGRLYAESEFDLAALDPIRSSLVEVGRSCVHPDHRDGAVIGLVWAGIARYMTDRGHAWLAGCCSLPLADGGALAAGAWDRVRTKHLAPEEYRVRPLLPWVPRPAAPAARTELPALLRGYLRLGAWVCGEPAHDVDFGVADLYVLLPMNRVDPRYLRHFLSLAPA

SEQ ID NO: 70 - protein, 246 aa, Mycobacterium avium
MIEAAQRLRYEVFTSTPGFALPSADGSGRDVDRFDEFCDHLLVRDDDTGELVGCYRMLAPAGAIAAGGLYTATEFDIRAFDPLRPSLVEMGRAVVRDGHRNGGVVLLMWAGILAYLDRYGYDYVTGCVSVPIGDADDAPPGSRLRGVRDFVVSRHGAPARYRVRPHRPVVVDGTALDDIPPPARPSVPALMRGYLRLGAQVCGEPAHDPDFGVGDFCVLLGKQDADTRYLKRLRSVSAAAELAGGR

SEQ ID NO: 71 - protein, 246 aa, Mycobacterium bovis; identical to SEQ ID NO: 67

SEQ ID NO: 72 - protein, 149 aa, Vibrio cholerae
MINWQCIPFCQLTTQQLYELLKLRVDVFVVEQTCPYPELDNKDTLNEVHHLLGYQDGELVACARLLPAGVSYPSVSLGRVATKASARGNGLGHQLLQTALEQCQNLWPQQSIEIGAQEHLREFYARYGFVATSEIYLEDGIPHIDMKRA

SEQ ID NO: 73 - protein, 242 aa, Xylella fastidiosa
MRMQGLDTYFITPQASILEQSPLLMLRRSLRKHDLNIPAATIQGPLLLDTMSTEAVLLIRGFQNEQFQRKQAYLLGEAQQALERELAIHERCAYFVAKRNDVIVGVLRLCPAPFEFERLLAHTGKTWPDFSLHVEVSRFVMANCECQTSTSMLLIVEACAWAMSHGYEGIVALCRPATRMIFERYGLSTVWPDYFCIPTRNNQRYALLSSRWQALILGATRASEAALARTRQTATRHDPIPS

SEQ ID NO: 74 - protein, 23 aa, Vibrio fischeri
SILDKTKVCEAIRLTISGSKSKA

SEQ ID NO: 75 - protein, 22 aa, Vibrio harveyi
LSDTQAVCEVLRLTVSGNAQQK

SEQ ID NO: 76 - protein, 22 aa, Vibrio anguillarum
LTGTQAVCEVLRLTVSGNAQQK

SEQ ID NO: 77 - protein, 140 aa, Tetrahymena thermophila
LLDFDILTNDGTHRNMKLLIDLKNIFSRQLPKMPKEYIVKLVFDRHHESMVILKNKQKVIGGICFRQYKPQRFAEVAFLAVTANEQVRGYGTRLMNKFKDHMQKQNIEYLLTYADNFAIGYFKKQGFTKEHGTLMECYIH

SEQ ID NO: 78 - protein, 154 aa, Homo sapiens
NEFRCLTPEDAAGVFEIEREAFISVSGNCPLNLDEVQHFLTLCPELSLGWFVEGRLVAFIIGSLWDEERLTQESLALHRPRGHSAHLHALAVHRSFRQQGKGSVLLWRYLHHVGAQPAVRRAVLMCEDALVPFYQRFGFHPAGPLTFTEMHCSL

SEQ ID NO: 79 - protein, 117 aa, Enterococcus faecium
MIISEFDRNNPVLKDQLERIAVAAVDQDELVGFIGAIPQYGITGWELHPLVVESSRRKNQIGTRLVNYLEKEVASRGGITIYLGTDHPYEFYEKLGYKIVGVLPGWKPDIWMAKTII

SEQ ID NO: 80 - DNA, 25 bp, Artificial sequence
  primer
ctctcggaatcatatgcttgaactg

SEQ ID NO: 81 - DNA, 29 bp, Artificial sequence
  primer
ctcgtagtagaacctcgagttatcagacc

SEQ ID NO: 82 - protein, 197 aa, Artificial sequence
  mutant of Pseudomonas aeruginosa
MIVQIGRREEFDKKLLGEMHKLRAQVFKERKGWDVSVIDEMEIDGYDALSPYYMLIQEDGQVFGCWRILDTTGPYMLKNTFPELLHGKEAPCSPHIWELSRFAINSGQKGSLGFSDCTLEAMRALARYSLQNDIQTLVTVTTVGVEKMMIRAGLDVSRFGPHLKIGIERAVALRIELNAKTQIALYGGVLVEQRLAV

SEQ ID NO: 83 - protein, 256 aa, Streptomyces coelicolor; identical to SEQ ID NO: 69

SEQ ID NO: 84 - protein, 278 aa, Ralstonia solanacearum
MRDLPTPTQPLIDALPSLSLGASNARRGLHRAPDAPARDKPVLAISWARHQDEVTEAQRLRYKVFAEEMGAHLASAGTELDVDMFDAVCDHLIVRDQQTLRVVGTYRVLRPDAAKRIGCLYSESEFDLVRLAHLRPKMVELGRSCVHRDYRSGSVIMALWAGLGEYMQRYGFESMLGCASVSMADGGHFAASLHRRFVEDGSLAPIEYHAFPRVPLPVDELNQTLEAEPPALIKGYLRLGSRICGAPAWDPDFNVADFLTLLRLSDINPRYARHFLRG

SEQ ID NO: 85 - protein, 272 aa, Burkholderia thailandensis
MRELPTPTLPLASLPLDLPRRRLPRAAETVTAEFRLRAAWARTEDELREAQRLRHSVFAEEMGAHVSGPAGLDVDPFDPYCDHLLVRDLDTLKVVGTYRVLPPHQAARVGRLYAEGEFDLSRLTHLRSKMVEVGRSCVHRDYRSGAVIMAMWGGLGTYMLQNGYETMLGCASVSMADGGHYAANLYQSLGDALTAPEYRAFPHTPLPVDELQTGVKVAPPPLIKGYLRLGAKICGAPAWDPDFNCADFLTLFRLSDINARYARHFLSDPLPR

SEQ ID NO: 86 - protein, 251 aa, Pseudomonas aeruginosa
MTQTAITREPVAGRRLKAERLNGARALREAQALRYRVFSAEFDAKLEGAEDGLDRDDYDRHCAHIGVRDLDSGALVATTRLLDHRAAERLGRFYSEEEFHLSGLDALHGPVLEIGRTCVAPEYRNGATIAVLWGELAEVLNEGGYRYLMGCASIPMRDGGMQAKAVMQRLRERYLCTDYLQAEPKNPLPPLDVPENLTAELPPLLKAYMRLGAKICGEPCWDPDFQVADVFILLKRDELCPRYARHFKAAV

SEQ ID NO: 87 - protein, 281 aa, Brucella melitensis
MSGLEAQQALFASNGDAIILGRIGSLEVRLANSRAAIEAAQELRFRVFFEEMGARKETIEAVEQRDADRFDTICDHLLVYDTALPVPEHQQIVGTYRLMRNEQAEKALGFYSADEYDVQRLKLSRPNLRLLELGRSCVKPEYRSKRTVELLWQGAWAYCRRHSIDVMFGCASFHGAVPAAHALGLSFLHHNCRATDDWDVRALPHRYLAMDLMPKEAINNKVALFSMPPLVKGYLRLGAMIGDGAVIDEAFGTTDVFIILPIERISSRYISYYGAEANRFV

SEQ ID NO: 88 - protein, 293 aa, Agrobacterium tumefaciens
MVAEIFNHDICENNVVISPRSETAQDNEGLFGRIGTLETRLARNEREIDAAQSVRYRVFVEEMKARLPAEAMRRKRDFDAWDSVCDHLLVLDKSIEGDSEDQIVGTYRLLRQETALANNGFYSASEFDIAGLVARHPGKRFMELGRSCVLPEYRTKRTVELLWQGNWAYAVKHRMDAMIGCASFPGVQPEAHALALSFLHHNCLAKGEWEAVALPELYHEMDLVPVEALNTRKALNAMPPLIKGYMRLGAMFGSGAVVDHAFNTTDVLVVLPVSSIAGRYISYYGGEAERING

SEQ ID NO: 89 - protein, 267 aa, Nostoc sp.
MEISYHHIKYPLRPPTIIKDFPILETDKYILKLAENEEELASIFRLRFEVFNVELGLGLADSNLTKMDQDEFDEICHHLMLISKLTGKTIGTYRMQTYKMASQGLGFDAADIFELKTIPESVLKVSVEVGRACIAKEYRSFQSLLLLWKGLADYLILNCSKYFFGCASLLTQCSWEAACAYHYFEQHKFIHKDILVFPHSQFYIDIPDKSNDVCRVDIPNILQAYLNVGAKICSLPAIDREFKTIDFLTIANIKEFTRWHYPNCLDK

SEQ ID NO: 90 - protein, 246 aa, Mycobacterium avium; identical to SEQ ID NO: 70

SEQ ID NO: 91 - protein, 246 aa, Mycobacterium bovis; identical to SEQ ID NO: 67

SEQ ID NO: 92 - protein, 213 aa, Mesorhizobium loti; identical to SEQ ID NO: 54

SEQ ID NO: 93 - protein, 220 aa, Mycobacterium avium
SGRDVDRFDEFCDHLLVRDDDTGELVGCYRMLAPAGAIAAGGLYTATEFDIRAFDPLRPSLVEMGRAVVRDGHRNGGVVLLMWAGILAYLDRYGYDYVTGCVSVPIGDADDAPPGSRLRGVRDFVVSRHGAPARYRVRPHRPVVVDGTALDDIPPPARPSVPALMRGYLRLGAQVCGEPAHDPDFGVGDFCVLLGKQDADTRYLKRLRSVSAAAELAGGR

SEQ ID NO: 94 - protein, 284 aa, Mycobacterium tuberculosis
  MISC_FEATURE (247)..(247): Xaa=any amino acid
MVEAAQRLRYDVFSTTPGFALPAAADTRRDGDRFDEYCDHLLVRDDDTGELVGCYRMLAPAGAIAAGGLYTATEFDVCAFDPLRPSLVEMGRAVVREGHRNGGVVLLMWAGILAYLDRYGYDYVTGCVSVPIGGDGETPGSRLRGVRDFILNRHAAPPQCQVYPYRPVRVDGRSLDDILPPPRPAVPPLMRGYLRLGARACGEPAHDPDFGVGDFCLLLDKDHADTRYLRRLRSVAAASEMVNDARXALPQSPNTPGCPAQRAASAAXVSATLRRCGGRWWCCG

SEQ ID NO: 95 - protein, 246 aa, Mycobacterium bovis; identical to SEQ ID NO: 67

SEQ ID NO: 96 - protein, 244 aa, Mycobacterium smegmatis
LIDAAQRLRHDVFTSEPGYALAGSTDGRDADRFDEYCDHLLVRDERSGELVGCYRMLPPPGAIAAGGLYTATEFDVTALDVLRPSLVEMGRAVVRQDHRNGAVVLLMWAGILAYLDHAGYDHVTGCVSVPVAGAAGEAPGAQIRGVRDFVRRRHAAPYTVHPYRPVVLDGRTLDDIAPPERVTVPALMRGYLRLGAQVCGEPAHDPVFGVGDFPALLDKRQADVRYLRRLRSASAAAHMTDGAA

SEQ ID NO: 97 - protein, 237 aa, Mycobacterium smegmatis
LIEAGQRLRREVLTDECGYTAAGTGFDADSFDDHCVHVLVRDNRTEELVGCARILPTGGVFATGGLYAAKSFDLTALDPLRLSLLEWGHAVVRADHRNGAVLMMMWSAILDYADRYGYDHLFGCITVPTHPLGSVPGAQVRAVRDFMRRDFATPDCYAVHPYRPVVIDGVPLDDMPLDTAAVSDSPVPALVRGYLRLGARVCGEPAHDPLFGVAHFPTLLRTGRFDGGSVLNRTDGW

SEQ ID NO: 98 - protein, 229 aa, Bordetella bronchiseptica
VEQIQRLRYDVFTEDMGAVFPQAQDGVEQDRFDQWCEHLMVRELDTGRVVGTYRILTPEKAREAGGYYSESEFDLSGLGALREQLVEVGRSCTHADYRNGAVIMLLWSGLAEYLRRGGYEYVLGCASVSLRDDGVTAAEVWRNVARHLDDPALPRVRPLHRYPIERLNSTLPARVPPLIKGYLKLGAKVCGEPAWDPDFNAADFPVLLSMAGMDERYRRHFGLDREARR

SEQ ID NO: 99 - protein, 229 aa, Bordetella parapertussis; identical to SEQ ID NO: 98

SEQ ID NO: 100 - protein, 210 aa, Erwinia stewartii; identical to SEQ ID NO: 1